SEQUENCE LISTING (2 sequences)

SEQ ID NO: 1 - protein, 893 aa, Homo sapiens
MTPPSRAEAGVRRSRVPSEGRWRGAEPPGISASTQPASAGRAARHCGAMSGARGEGPEAGAGGAGGRAAPENPGGVLSVELPGLLAQLARSFALLLPVYALGYLGLSFSWVLLALALLAWCRRSRGLKALRLCRALALLEDEERVVRLGVRACDLPAWVHFPDTERAEWLNKTVKHMWPFICQFIEKLFRETIEPAVRGANTHLSTFSFTKVDVGQQPLRINGVKVYTENVDKRQIILDLQISFVGNCEIDLEIKRYFCRAGVKSIQIHGTMRVILEPLIGDMPLVGALSIFFLRKPLLEINWTGLTNLLDVPGLNGLSDTIILDIISNYLVLPNRITVPLVSEVQIAQLRFPVPKGVLRIHFIEAQDLQGKDTYLKGLVKGKSDPYGIIRVGNQIFQSRVIKENLSPKWNEVYEALVYEHPGQELEIELFDEDPDKDDFLGSLMIDLIEVEKERLLDEWFTLDEVPKGKLHLRLEWLTLMPNASNLDKVLTDIKADKDQANDGLSSALLILYLDSARNLPSGKKISSNPNPVVQMSVGHKAQESKIRYKTNEPVWEENFTFFIHNPKRQDLEVEVRDEQHQCSLGNLKVPLSQLLTSEDMTVSQRFQLSNSGPNSTIKMKIALRVLHLEKRERPPDHQHSAQVKRPSVSKEGRKTSIKSHMSGSPGPGGSNTAPSTPVIGGSDKPGMEEKAQPPEAGPQGLHDLGRSSSSLLASPGHISVKEPTPSIASDISLPIATQELRQRLRQLENGTTLGQSPLGQIQLTIRHSSQRNKLIVVVHACRNLIAFSEDGSDPYVRMYLLPDKRRSGRRKTHVSKKTLNPVFDQSFDFSVSLPEVQRRTLDVAVKNSGGFLSKDKGLLGKVLVALASEELAKGWTQWYDLTEDGTRPQAMT

SEQ ID NO: 2 - DNA, 5957 bp, Homo sapiens
agtatccaccccgcccgctcccggtgacgtgccagccccaggcccacgccgctcccgccccgcgtgatgacgccaccgtcccgggcggaggcgggcgtgcggcggagccgcgtcccctcagaggggcgctggcgcggggctgagccgcccgggatcagcgcgagcacccagcccgcctcggccgggagggcagcgcggcactgcggggcgatgagcggcgcccggggcgagggcccggaggcgggcgccggcggggctgggggccgcgcggcgcctgagaaccccgggggcgtgctgagcgtggagctgcccgggctgctggcgcagctggcgcggagcttcgcgctgctgctgcccgtgtacgcgctgggctacctggggctcagcttcagctgggttctcctcgcgctcgcgctgctcgcctggtgtcgccgcagccgcggcctcaaggccctgcgcctgtgccgcgcgctggcgctgctggaagacgaggagcgcgtcgtgcgcctgggggtgcgcgcctgcgacctgcccgcctgggttcattttccagacactgaaagagcagaatggctaaataagactgtaaaacacatgtggcctttcatttgccaatttatagagaagttgtttcgagaaactatagaaccagccgtgcggggagcaaacacccaccttagcacctttagtttcacgaaggtcgacgtgggccagcagcccctcaggatcaatggtgttaaggtatacactgaaaatgtagacaaaaggcaaattattttggaccttcagattagttttgtaggaaattgtgagattgatttggagatcaaacgatatttttgtagagctggtgtgaaaagtatccagattcatggtaccatgcgggtgatcctggaaccgttgattggagatatgcccttagttggagctttgtctatcttcttccttaggaaaccacttttagaaattaactggacaggactgacgaatcttctggatgtccctggattgaatggtttatcagatactatcattttggatataatatcaaactatctggtgcttcccaatcgaatcaccgttccacttgtcagtgaagttcaaatagctcagttgcggtttcctgtaccaaagggtgttctaaggatacattttattgaagctcaggatcttcaggggaaagacacttaccttaagggacttgtcaagggaaagtcagacccctatggaatcattagagttggcaaccaaatcttccaaagcagagtcatcaaggagaacctcagtccaaagtggaatgaagtctatgaggctttagtgtatgaacatcctggacaagaattagagattgagctctttgatgaagacccagacaaggatgactttttaggaagtcttatgattgacctcattgaagttgaaaaggagcgccttttagatgaatggttcactctggacgaggttcccaaggggaagctacacttgagactggagtggctcacgttaatgccaaatgcgtcaaacctcgacaaggtgctaacagacatcaaagctgacaaagaccaagccaacgatggtctttcctctgcattgctgatcttgtacttggattcagcaaggaaccttccgtcagggaagaaaataagcagcaacccaaatcctgttgtccagatgtcagttgggcacaaggcccaggagagcaagattcgatacaaaaccaatgaacctgtgtgggaggaaaacttcactttcttcattcacaatcccaagcgccaggaccttgaagttgaggtcagagacgagcagcaccagtgttccctggggaacctgaaggtccccctcagccagctgctcaccagtgaggacatgactgtgagccagcgcttccagctcagtaactcgggtccaaacagcaccatcaagatgaagattgccctgcgggtgctccatctcgaaaagcgagaaaggcctccagaccaccaacactcagctcaagtcaaacgtccctctgtgtccaaagaggggaggaaaacatccatcaaatctcatatgtctgggtctccaggccctggtggcagcaacacagctccatccacaccagtcattgggggcagtgataagcctggtatggaagaaaaggcccagccccctgaggccggccctcaggggctgcacgacctgggcagaagctcctccagcctcctggcctccccaggccacatctcagtcaaggagccgacccccagcatcgcctcggacatctcgctgcccatcgccacccaggagctgcggcaaaggctgaggcagctggaaaacgggacgaccctgggacagtctccactggggcagatccagctgaccatccggcacagctcgcagagaaacaagcttatcgtggtcgtgcatgcctgcagaaacctcattgccttctctgaagacggctctgacccctatgtccgcatgtatttattaccagacaagaggcggtcaggaaggaggaaaacacacgtgtcaaagaaaacattaaatccagtgtttgatcaaagctttgatttcagtgtttcgttaccagaagtgcagaggagaacgctcgacgttgccgtgaagaacagtggcggcttcctgtccaaagacaaagggctccttggcaaagtattggttgctctggcatctgaagaacttgccaaaggctggacccagtggtatgacctcacggaagatgggacgaggcctcaggcgatgacatagccgcagcaggcaggaggcgtcctcttcagcgtagctctccacctctacccggaacacaccctctcacagacgtaccaatgttatttttataatttcatggatttagttatacataccttaatagttttataaaattgttgacatttcaggcaaatttggccaatattatcattgaattttctgtgttggatttcctctaggatttcgccagttcctacaacgtgcagtagggcggcggtagctcttgtgtctgtggactctgctcagctgtgtccgtaggagtcggatgtgtctgtgctttattatggccttgtttatatatcactgaggtatactatgccatgtaaatagactattttttataatctttacatgctggtttaaattcagaaggaaatagatcaaggaaatatatatattttcttctaaaacttattaaattcgtgtgacaaataatcattttcatcttggcagcaaaaagttctcagtgacctattttgtggtgtttctttttgaaaagaaaagctgaaatattattaaatgctagtatgtttctgcccattatgaaagatgaaataaagtattcaaaatattaacattttcataaatataaggatgtattattgagaagtaagttgaagggcttataaggaaaaatgttttataactgagtaatatattaagagaattgtcatggttcataaatcacattatgctaatctgaaatttcttacataaaaatgaagtgtcttatgtttattttaattgctgttgtaacttactcatgaaacagtatacagacaccttgtacttttcctcaactgtaagagcagactttcaatgttagcaattaagctgttgtcaaacaatcagtcatgagctttgttaattttcaatgttttcccagcctataaaaaaaggaaggtacacgttgtccttttaaaggttgtgaggtagattggagtgagtagacaggatattgcattaaaaattgaaagctcgatctcattattgtcaggaacccccagtgtgacctcacacataggatgtgggacctttgagccgatgtgcactggccaccaccagggttgggggcgccacagctgcgagcccggcccctgctgttctcagcagccacttcccaggctgcctcactttatgccatgactgaccttaatattgggatattgttatgcaatttttatcctgtttagactgttaaaagcaggtttctgtacttaagagtcccccagacctcctgtgaggtgaggctctgttgcagtgtcgtaggctgtgtgtgtctgtaaagaaagaatgcacatatgtagacgattaagtgtatattataagctatatgctgaaaatggcttccatagccatgagaacatcttaaaactatgtgtaaatatattaaggaaagtatagctttgtaatttaaattggagcttttagcttgtttcatggaactatacaacttgtgggtaactcacatgaccaaacaaaccaaagtgcctgtgacggggcgggtggcgtctacccaccctcccttctagcagattcttattttgtttgaatttataaacaaggctggtggctgtctacccaccctcccttctagcagattttgattttgtttcaatttataacttacactttgaaccatgggtttacttataatggagtctgtagcttcacagcatatttcatgtaatcataaagaccagtatattccctcctgctgaatgacatgcgactgtaaagcctctttataaaccatttccaatgttagtatataggattatttgggaagcgtatcaatacctttatagacaaatacgaacatgtatgcacacaaaacatttaactatggtatttatggaagacaggtaacaacattaaatctagttgctttcccttagtattagatttgttgagggttttttaaaaatcaggtctgttgaaagtcttctgtcataatctataaagcagcagcactcatggaaattgtagcatgccagtaatttttaccaacatcccatacatctgagttctgcagtccagtgtgtaatccgctccatgtgtattttgcttaatggaatgctttatttaagcacttaggcagagtagacacaattaaaggtacaaagcccagaggaagtggtagagcagcaccgtgcctgccctgaggcagtggagtcagtagcgctgtccccagggccttgagtgcctggaggtgcttggcctccagtagctgcctccattctcttttaaaaaaagggggtgattctgaggcactgaagtgcctcccagatgtggaggagtgaagccaccatcgaggccacactcagcactccaggatcccagcgatgtcagacactcttgagttgtcaaaacgttaattttcagttttaaataatcagtttatctaagaaaagggaattttaacttttctaccttgagccaagccaatgaagggaaaattaattaacttagtaaatttgaagtgcagctctgttagctcgtacatgtgggttcttatcctgatcctgtgccttaaagtaggaaggtgtttccaagttcagattaaaatagaagcagctggccgggtgcggtggctcacgcctgtaatcccagcactttgggaggccgaggcgggcggatgacctgaggtcaggagttcaagaccagcctggccaacatggagaaaccccatctttactaaaaatacaaaaattagccgggcgtggtggtggagcgcacgcctgtagtcccagctactcgggaggctgaggcaggagaattgcttgaacccaggaggcggaggttgcagtgagccaagatcgcaccactgcactccagcctgggcaacagagtgagactccgtctcaaaaaataaataaaatagaagcagccttgtaactgtatttaccatgataatatattctgcacggtaagaattccttttacagacattctttatcaagaggtcggcccttctttttcaggcacataagccaaatgcaggcctgtgtgtagctgtgtgttttttctgtggttgccgcatttattccacctccagctggaccccccactgcaaatagagaacagcggtgggggatgggggttaaaaagtagagaacctcctttctgttcaactaatttcacgtgacagtgcatgtatttattcaataaaacctttatgttagctc

(19) United States
(12) Patent Application Publication
(10) Pub. No.: US 2018/0066318 A1
(43) Pub. Date: Mar. 8, 2018

(54) METHODS OF CHARACTERIZING E-SYT2 INHIBITORS, E-SYT2 INHIBITORS, AND METHODS OF USE

(71) Applicants: INSTITUT PASTEUR, Paris (FR); UNIVERSITE PARIS DIDEROT - PARIS 7, Paris (FR)

(72) Inventors: Robert WEIL, Saint Cloud (FR); Nassima MESSALI, Paris (FR); Helena SOARES, Lisboa (PT); Satoh-Takayama NAOKO, Paris (FR); Andromahi TRIVELLAS, Paris (FR); James DI SANTO, Paris (FR); Andrés ALCOVER, Montrouge (US)

(21) Appl. No.: 15/554,424

(22) PCT Filed: Mar. 4, 2016
(86) PCT No.: PCT/IB2016/000792

§ 371 Date: Aug. 29, 2017

Related U.S. Application Data
(60) Provisional application No. 62/128,701, filed on Mar. 5, 2015.

(51) Int. Cl. C12Q 1/68; G01N 33/574; A01K 67/027

(52) U.S. Cl. CPC C12Q 1/6886; G01N 33/57407; A01K 67/0276; A01K 2217/077; G01N 2500/10; A01K 2267/0387; C12Q 2600/118; C12Q 2600/156; A01K 2227/105

(57) ABSTRACT

The invention provides methods of identifying an E-SYT2 modulator. The methods may comprise providing a cell that expresses E-SYT2; contacting the cell with a candidate chemical entity; and characterizing recruitment of at least one of Carma1, BcllO, NEMO, and PKC9 to the immunological synapse (IS). The invention also provides E-SYT2 modulators, including inhibitors, identified using the disclosed methods. The invention provides methods of inhibiting NF-κB activity in a cell comprising contacting the cell with an E-SYT2 inhibitor. The invention provides methods comprising providing a sample from a patient suspected of having or at risk of having a MALT-lymphoma or an ABC-DLBCL-lymphoma; and screening the sample to identify the presence and/or absence of a gain of function E-Syt2 mutation in the sample. The invention also provides genetically modified mammals comprising one or two loss of function alleles of E-Syt2.

### INTRODUCTION

T lymphocytes are activated by the presentation of an antigenic peptide in association with the major histocompatibility complex (MHC) present at the cell surface of antigen-presenting cells (APC) to the T cell receptor. This activation process is characterized by the formation of a structure called the immune synapse (IS), which is at the contact between the APC and the T cell. The immune synapse is a dynamic platform for local signaling and its stability is critical for the initiation of the immune response¹. Stimulation of the T cell receptor activates transcription factors such as NFAT (nuclear factor of activated T-cells) and NF-κB (nuclear factor kappa B), which participate in long-term biological responses such as proliferation, survival and differentiation. In normal resting cells, NF-κB proteins are sequestered in the cytoplasm through their interaction with the IκB inhibitors. T-cell receptor (TCR) stimulation induced a signaling cascade that converges on the IKK kinase complex formed by the IKKα and IKKβ kinases and the regulatory subunit NEMO (NF-κB essential modulator)¹. This kinase complex is responsible for IκB phosphorylation and their subsequent degradation, allowing nuclear translocation of NF-κB and activation of its target genes.

The signal-activation cascade that results from the engagement of the TCR and CD28 costimulatory molecules is initiated by the rapid activation of tyrosine kinases of the Src and Syk families, subsequent phosphorylation of immunoreceptor tyrosine-based activation motifs (ITAMs) contained within the cytoplasmic domains of the invariant subunits of the TCR complex and binding of cytosolic effectors to phosphorylated adaptors. A key substrate for TCR-coupled protein tyrosine kinases is phospholipase C-gamma 1 (PLC-γ1), which hydrolyzes the lipid PI(4,5)P2 into two second messengers, the membrane diacylglycerol (DAG) and the soluble inositol (1, 4, 5) triphosphate (IP3). DAG induces PKC activation by abrogating an autoinhibitory association between the PKC pseudo-substrate and substrate-binding domain². This action can be mimic by phorbol-12, 13-dibutyrate (PDBu) and phorbol myristate acetate (PMA)³. PKCθ is a Ca2+-independent nPKC isoform predominantly expressed in T lymphocyte. Different studies suggest that Lck mediates PKCθ phosphorylation and membrane translocation^{4,5}, and induces the formation of a tripartite PKCθ/Lck/CD28 interaction at the membrane⁶⁻⁸. PKCθ is upstream of CARMA1, a member of the membrane-associated guanylate kinase (MAGUK) family of proteins, in the pathway leading to NF-κB activation. Phosphorylation of CARMA1 by PKCθ in response to TCR/CD28 stimulation modifies its conformation allowing its association with a constitutively associated dimer formed by the B-cell lymphoma/leukemia 10 (BCL10) and mucosa-associated lymphoid tissue 1 (MALT1) protein, leading to the assembly of the CBM complex⁹. The activation of the NEMO/IKK complex requires the recruitment of NEMO to polyubiquitinated BCL10 and/or MALT1 in addition to NEMO ubiquitination and IKK kinases phosphorylation by the serine/threonine kinase transforming growth factor β-activated kinase 1 (TAK1)^{10,11}. Interestingly, CARMA1 is required for the K63-linked ubiquitination of NEMO but is dispensable for phosphorylation of the IKK kinases by the serine/threonine kinase transforming growth factor β-activated kinase 1 (TAK1)¹². In addition, a fourth protein, ADAP, plays an important role for the assembly of the CARMA1-BCL10-MALT1 (CBM) complex, recruitment of TAK1 and consequently for the phosphorylation of IKKα/β¹³⁻¹⁵.

Importantly, enzymatic activity of Caspase 8 and paracaspase MALT1 are required for TCR-induced NF-κB activation¹⁶⁻¹⁸ and the protease activity of MALT1 is controlled by monoubiquitination¹⁹. To better characterize the physiological functions of PKCθ and of the CBM complex, a number of genetically engineered mouse models have been generated¹. Overall, most of these mice present the same type of defects. Different reports showed that mice deficient for some of these proteins present an increase in cell death of early thymocytes^{20,21}. TCR-induced upregulation of CD25 and/or CD69 and/or CD44 expression in mature T cells was also reduced in some cases²¹⁻²⁵. In addition, mature T cells present also defective TCR-mediated lymphocyte proliferation mainly due to the reduced production of IL-2 probably as a consequence of the impairment of NF-κB activation²⁰⁻²⁶.

In analogy to neurons, TCR stimulation results in the assembly of signaling molecules in supramolecular clusters. MAGUK proteins play important roles in synaptic development and in IS signaling. Over the past few years, several groups have shown that PKCθ, CARMA1, BCL10, MALT1, ADAP and the NEMO/IKK complex are localized in the IS following TCR stimulation²⁷⁻³². The molecular mechanisms responsible for the recruitment and activation of the CBM complex to the immunological synapse are not fully understood. We identified Extended-Synaptotagmin 2 (E-Syt2) (alternative names KIAA1228, CHR2SYT or FAM62B) has a potent candidate for targeting CARMA1 in the IS. E-Syt2 belongs to a family of homologous proteins referred as Extended-Synaptotagmins, because of their similarities to yeast proteins called tricalbins, resembling the neuro synaptic vesicle proteins Synaptotagmins³³. E-Syts proteins were identified by searching for proteins containing C2 domains and a transmembrane region³³. These C2 domains were originally discovered in protein kinase C isoforms. E-Syt2 contains three C2 domains referred as C2A, C2B and C2C. Its C2A domain interacts with phospholipids in a Ca2+-dependent manner^{33,34} and its C2C domain mediates its plasma membrane recruitment^{33,35}. Indeed, It has been recently shown that E-Syts and their homologs, the tricalbins, are endoplasmic reticulum anchored proteins that mediate contacts with the plasma membrane and that concerning E-Syt2, its C2C domain is required for this function^{35,36}. E-Syt2 has been also implicated in the endocytic trafficking of FGFR that is required for Erk activation and in the induction of the mesoderm³⁷. Interestingly, E-Syt2 C2C domain interacts with p21-activated kinase 1 (PAK1) and suppresses its activation, leading probably to actin depolymerisation in the vicinity of clathrin-coated pits during FGFR endocytosis³⁸.

NF-κB transcription factors can be uncoupled from their normal regulation and promote tumorigenesis in different ways. For example, mutations of the key components of the antigen-mediated signaling pathways are associated with constitutive CBM-mediated signaling, and with the development of particular subtypes of human B-cell lymphomas, including mucosa-associated lymphoid tissue (MALT) and diffuse large B-cell (DLBCL) lymphomas. Constitutive anti-apoptotic NF-κB signaling is a hallmark of ABC-DLBCL. Target directed approaches for ABC-DLBCL therapy have largely focused on the inhibition of upstream protein kinase. However, most oncogenic mutations in ABC-DLBCL occur further downstream revealing that these kinases may not be optimal targets. Indeed, approximately 10% of ABC-DLBCLs harbor activating missense mutations within the coiled-coil domain of CARMA1 and expression of these mutants leads to constitutive and enhanced antigen receptor-dependent activation of NF-κB.

There is a need in the art to identify additional components and mechanisms of the signaling pathways that mediate NF-κB signaling in human B-cell lymphomas, including mucosa-associated lymphoid tissue (MALT) and diffuse large B-cell (DLBCL) lymphomas, including ABC-DLBCL. There is also a need to develop methods and reagents to identify molecular entities that are useful to modulate NF-κB signaling in human B-cell lymphomas, including mucosa-associated lymphoid tissue (MALT) and diffuse large B-cell (DLBCL) lymphomas, including ABC-DLBCL. There is also a need for methods of using such molecular entities to treat human B-cell lymphomas, including mucosa-associated lymphoid tissue (MALT) and diffuse large B-cell (DLBCL) lymphomas, including ABC-DLBCL. There is also a need to identify molecular markers for characterization of human B-cell lymphomas, including mucosa-associated lymphoid tissue (MALT) and diffuse large B-cell (DLBCL) lymphomas, including ABC-DLBCL. The inventions disclosed herein meet one or more of these needs and/or other needs.

### SUMMARY

This disclosure proposes that E-Syt2 plays a pivotal role in antigen signaling by controlling the recruitment of the CBM complex and PKCθ at the IS and consequently NF-κB activation. It was observed that CD4+ T cells from E-Syt2 KO mice present an increase in TCR-mediated CD4+ T cell death that could be related to the impairment of NF-κB activation and that aged KO mice present important inflammatory skin lesions.

Based in part on the data presented in the Examples, the inventors have identified Extended-Synaptotagmin 2 (E-Syt2) as a protein that interacts with CARMA1 and shown that it orchestrates antigen signaling by controlling the formation of the CBM complex at the immune synapse. Based in part on the data presented in the Examples, the inventors have also demonstrated that CD4+ T cells from mice lacking E-Syt2 expression present an increase cell death in response to TCR stimulation. In part based on these data the inventors have been able to show that E-Syt2 orchestrates the recruitment of E-Syt2 to the immune synapse to ensure cell survival. E-Syt2 is probably the most upstream component of the CBM pathway. Based in part on these findings, the inventors herein provide new methods and reagents to modulate the activation of T-lymphocytes, initiated upon triggering of the T-cell antigen receptor by MHC-bound antigen in physiological conditions and in aberrant lymphocyte proliferation that occurs in certain lymphomas. This invention also proposes that E-Syt2 is critical for the survival of ABC-DLBCL and/or that mutations of E-Syt2 might be associated with these lymphomas. Accordingly, this invention provides methods and reagents to identify molecular entities that are useful to modulate NF-κB signaling in human B-cell lymphomas, including mucosa-associated lymphoid tissue (MALT) and diffuse large B-cell (DLBCL) lymphomas, including ABC-DLBCL. This invention also provides methods of using such molecular entities to treat human B-cell lymphomas, including mucosa-associated lymphoid tissue (MALT) and diffuse large B-cell (DLBCL) lymphomas, including ABC-DLBCL. This invention also provides molecular markers for characterization of human B-cell lymphomas, including mucosa-associated lymphoid tissue (MALT) and diffuse large B-cell (DLBCL) lymphomas, including ABC-DLBCL.

In a first aspect, the invention provides methods of identifying an E-SYT2 modulator. The methods may comprise providing a cell that expresses E-SYT2; contacting the cell with a candidate chemical entity; and characterizing recruitment of at least one of CARMA1, BCL10, NEMO, and PKCθ to the immunological synapse (IS). In some embodiments recruitment of at least one of CARMA1, BCL10, NEMO, and PKCθ to the IS is reduced in the presence of the candidate chemical entity, and the candidate chemical entity is thereby identified as an inhibitor of E-SYT2 activity, i.e., as an E-SYT2 inhibitor. In some embodiments the inhibitor of E-SYT2 activity binds to E-SYT2 protein to inhibit E-SYT2 protein function. In some embodiments the inhibitor of E-SYT2 activity inhibits expression of E-SYT2. In some embodiments the cell is a T cell.

The invention also provides E-SYT2 modulators, including inhibitors, identified using the disclosed methods.

In another aspect, the invention provides methods of inhibiting NF-κB activity in a cell comprising contacting the cell with an E-SYT2 inhibitor. In some embodiments contacting the cell with the E-SYT2 inhibitor reduces recruitment of at least one of CARMA1, BCL10, NEMO, and PKCθ to the immunological synapse (IS) in the cell. In some embodiments the E-SYT2 inhibitor binds to E-SYT2 protein to inhibit E-SYT2 protein function. In some embodiments the E-SYT2 inhibitor inhibits expression of E-Syt2. In some embodiments the cell in cultured in vitro. In some embodiments the cell is in a patient and the method comprises administering the E-SYT2 inhibitor to the patient. In some embodiments NF-κB is constitutively active in the cell in the absence of the E-SYT2 inhibitor. In some embodiments the cell is a T cell. In some embodiments the cell is a B cell.

Accordingly, the invention also relates to the use of a E-SYT2 modulator, especially a E-SYT2 inhibitor, as disclosed or defined herein, for inhibiting NF-κB activity in a cell, especially in a patient in need thereof, and/or reducing recruitment of at least one of CARMA1, BCL10, NEMO, and PKCθ to the immunological synapse (IS) in said cell. The invention is also directed to a E-SYT2 modulator, especially a E-SYT2 inhibitor, as disclosed or defined herein, for use for treating lymphoma or its symptom(s), in particular a MALT-lymphoma or a ABC-DLBCL-lymphoma, especially when these diseases are associated with increased NF-κB activity. According to a particular embodiment, the said E-SYT2 modulator further inhibits NF-κB activity in lymphoma cells and/or reduces recruitment of at least one of CARMA1, BCL10, NEMO, and PKCθ to the immunological synapse (IS) in said cells.

In another aspect, the invention provides methods of providing a sample from a patient suspected of having or at risk of having a MALT-lymphoma or an ABC-DLBCL-lymphoma; and screening the sample to identify the presence and/or absence of a gain of function E-SYT2 mutation in the sample. According to a particular embodiment, such a method is an in vitro method. In some embodiments, if the sample comprises a gain of function E-SYT2 mutation the patient is diagnosed as having a MALT-lymphoma or a ABC-DLBCL-lymphoma. In some embodiments, if the sample comprises a gain of function E-SYT2 mutation the patient is diagnosed as having an increased risk of having and/or developing a MALT-lymphoma or a ABC-DLBCL-lymphoma. In some embodiments the screening comprises analysing a nucleic acid present in the sample. In some embodiments the screening comprises performing a hybridization and/or polymerization assay on the nucleic acid. In some embodiments the screening comprises analysing the activity of E-SYT2 protein in the sample.

In another aspect, the invention provides genetically modified mammals comprising one or two loss of function alleles of E-Syt2. In some embodiments the genetically modified mammals develop dermatitis.

In another aspect, the invention provides genetically modified mammals comprising two copies of a conditional loss of function allele of E-SYT2, wherein the conditional allele is recombined in the thymus of the genetically modified mammal such that the thymus of the mammal comprises cells that do not express functional E-SYT2.

### BRIEF DESCRIPTION OF THE TABLE AND DRAWINGS

Table 1: Interacting proteins of CARMA1 SH3 domain (two hybrid screen). Legend below.

FIGS. 1a-1d: binding of CARMA1 SH3 domain to E-Syt2. GST pull-down assays show direct interaction between E-Syt2 and SH3 domains. Schematic representation of CARMA1 (a) and E-Syt2 (b) mutant constructs. (c) upper panel: pull-down assays of the interactions between CARMA1 SH3 domain (GST-SH3c), Vav SH3 domain (GST-SH3v) or GST and in vitro translated HA-tagged E-Syt2, or NEMO in TNT® reticulocytes lysate systems. Ten percent of the input TNT lysates was also run on the PAGE and immunoblot with anti-HA to determine the relative binding capacity. Bottom panel: shows E-SYT2 localization to the IS. Jurkat T cells were incubated with Raji B cells and coated with medium alone or with SEE. At 10 minutes, cells were fixed and E-SYT2 localized by immunofluorescence and confocal microscopy, using anti-A-SYT2 antibody, as described in the experimental procedure. The corresponding Nomarski images are shown on the bottom panels. Scale Bars represent 1 μm. (d) ESYT2 localized in microclusters Jurkat cells were settle onto glass coverslips coated either with anti-CD3, anti-CD28 or both and fixed 5 min of stimulation. The images of E-Syt2, PKCθ and TCT staining were taken by confocal microscopy.

FIGS. 2a-2c: E-SYT2 interacts with CARMA1 at the Immune synapse. (a) HEK293T cells were transfected with VSV-CARMA1, myc-E-SYT2, PKCθ wt, DN and DA as indicated. Total lysates were immunoprecipitated with anti-VSV and western blot analysis was performed using anti-VSV, anti-myc antibodies, anti-PKCθ and anti-α Tubulin antibodies. (b) Jurkat cells were stimulated for the indicated periods of time with Phorbol 12-myristate 13-acetate (PMA) and lonomycin, lysed and immunoprecipitated with Ab raised against CARMA1. The western blots were probed with anti-CARMA1, anti-E-SYT2, anti-BCL10, anti-P IκBα, anti-IκBα and anti-α Tubulin. (c) E-SYT2 colocalized with CARMA1 at the Immune synapse. Jurkat T cells were incubated with Raji B cells and coated with medium alone or with SEE. At 10 minutes, cells were fixed and E-Syt2 and CARMA1 localized by immunofluorescence and confocal microscopy, using anti-A-SYT2 and anti-CARMA1 antibodies, as described in the experimental procedure. The corresponding Nomarski images are shown on the left panels and merging of the staining is shown on the right. Scale Bars represent 1 μm.

FIGS. 3a-3c: CARMA1 SH3 domain is required for the coclustering of CARMA1 with E-Syt2 at the T/APC interface. (a) Schematic representation of CARMA1 domain organization and mutations used in this study. (b) PKCθ and E-SYT2 are recruited at the immune synapse in CARMA1-deficient JPM50.6 cells (c) CARMA1 SH3 domain allows the interaction of CARMA1 at the immune synapse and its colocalization with E-SYT2. JPM50.6 CARMA1-deficient cells were mixed with Raji cells pulsed with SEE and left for 10 min at 37° C. Conjugates were fixed and stained with anti-E-CARMA1, anti-E-SYT2, anti-PKCθ and anti-TCR followed by Cy3 (red), Alexa 488 (green) or Cy5 (Blue)-coupled secondary Abs. The corresponding Nomarski images are shown on the right panels and merging of the staining is also shown on the right. Scale Bars represent 1 μm.

FIGS. 4a-4f: Expression, localization and heterodimerization of E-Syt1 and E-Syt2 in Jurkat cells. (A) Immunofluorescence images showing the localization of E-Syt1 and E-Syt2. (B) Jurkat cells were stimulated with anti-CD3 and anti-CD28 antibodies for the indicated time. Co-immunoprecipitation of endogenous E-Syt1 with E-Syt2 was determined by western blot using anti-E-Syt1 and anti-E-Syt2 antibodies. The levels of phosphorylation of IkBoc and the expression of a Tubulin were measured in whole cell lysates (WCE) as controls of stimulation and loading respectively. (C) Expression of E-Syt1 and E-Syt2 in Jurkat cells transfected with either a non-targeting siRNA (NT), an E-Syt1- or E-Syt2-directed siRNA. (D) (E) (F) Jurkat cells transfected with the indicated siRNA were incubated with SEE-loaded Raji B cells. After 10 minutes, cells were fixed and TCRZ, LCK, CARMA1, PKC6 and E-Syt2 were detected by immunofluorescence and confocal microscopy. Scale bars represent 5 μm. The graphs represents the fluorescence intensity of E-Syt2, CARMA1, PKC6, LCK or TCR at the IS in Jurkat cells transfected with a control E-Syt1- or E-Syt2-targeting siRNA of at least 20 conjugates for each group, each point represents value obtained from single cells. ****, P<0.0001; ***, P=0.0001; **, P<0.01; *, P<0.05; Mann-Whitney test using Prism software.

FIGS. 5a-5d: E-SYT2 is required for the localization of CARMA1, BCL10, PKCθ and ADAP at the Immune synapse and microclusters. (a) Jurkat cells transfected with either an irrelevant siRNA (Ctrl) or an E-SYT2 directed siRNA were incubated with Raji B cells and prepulsed with SEE. After 10 minutes, cells were fixed and CARMA1, BCL10, TCR localized by immunofluorescence and confocal microscopy, using anti-CARMA1, anti-BCL10 and anti-TCR antibodies. The corresponding Nomarski images are shown on the right panels and merging of the staining is also shown on the right. Scale Bars represent 1 μm. (b) Representative images of conjugates formed in the same condition as panel A, stained with anti-PKCθ and anti-Lck. (c) Jurkat cells were settle onto glass coverslips coated either with anti-CD3, anti-CD28 or both and fixed 5 min of stimulation. The images of E-SYT2, PKCθ and TCR staining were taken by confocal microscopy. Scale Bars represent 1 μm. (d) E-SYT2 is required for the localization of ADAP to MCs. Jurkat cells transfected with either an irrelevant siRNA (Ctrl) or an E-SYT2 directed siRNA were incubated with Raji B cells and prepulsed with SEE. After 10 minutes, cells were fixed and ADAP and TCR localized by immunofluorescence and confocal microscopy, using anti-ADAP and anti-TCR antibodies. Merging of the staining is shown on the right.

FIGS. 6a-6o: Flow cytometric analyses of lymphocytes populations in E-Syt2 KO mice. (A) Photograph of representative spleen from Control (Ctr), E-Syt1′-(KOt) and E-Syt2ⁿ^{_{O}}^{x/n}^{_{O}}^{x} CD4-Cre (KOc) 6 weeks old mice. (B) Total number of splenocytes of Ctr, KOt and KOc mice. (C) Total number of CD3+ cells in the spleen. (D) B and T cell ratio in the spleen. (E) CD4⁺, CD8⁺ and (F) CD62L⁺CD44⁺ T cells in the spleen. (G) CD25⁺FoxP3⁺ T cells in the spleen and thymus. (H) and (I) Detection of apoptotic cells using Annexin V in CD4+ lymphocytes purified from the spleen of Ctr or E-Syt2^{-/-} mice and stimulated with anti-CD3 and anti-CD28-coated beads with or without IL2 for 72 hours. (J) Supernatants of CD4⁺ T cells stimulated during 24 h with anti-CD3 and anti-CD28 antibodies or PMA and ionomycin were collected and the levels of secreted IL-2 were determined by ELISA. Flow cytometric analyses of E-Syt2-deficient thymus, and lymph node cells. (K) Total number of Thymocytes. (L) Total cell number in Lymph Nodes. (M) total CD3⁺ cells in Lymph Nodes. (N) Total number of CD4⁺, CD8⁺ cells in the Thymus and Lymph Nodes. (O) Number of total CD25+FoxP3+ T cells in the thymus and Lymph Nodes. ****, P<0.0001; ***, P=0.0001; **, P<0.01; *, P<0.05; T Student test using Prism software.

FIG. 7: E-SYT2 plays an important role for the localization of Endoplasmic Reticulum in the IS.T cell-APC conjugates were formed as described. Localization of E-SYT2, Calnexin and TCR are shown. Merging of the staining is shown on the right. Scale Bars represent 1 μm.

FIGS. 8a-8d: (a) Genomic E-Syt2 sequence and construction of the neomycin resistance (Neo) insertion vector. E-Syt2 exons are shown as boxes, selection gene cassettes, PCR primer positions, LoxP sites, FRT sites are indicated. (b) PCR genotyping from tail biopsies genomic DNA. (c) Western Blot analyzing of thymus, lymph nodes and spleen protein lysates from E-Syt2+/+, −/+ and +/+ mice using E-SYT2 antibodies. (d) Western Blot analyzing of thymus, CD4⁺ cells purified from lymph nodes and spleen and heart lysates from control and E-Syt2^{flox/flox} CD4-Cre mice (KOc) using E-Syt2 antibodies and anti-α Tubulin as control.

FIGS. 9a-9c: Deletion of E-SYT2 reduces splenocytes number and T cell activation. Average total counts of T cells in the thymus (a) and in the spleen (b). Number of total CD4+ T cells (c) and of the CD4+ T cells subsets CD69 and CD62L in the spleen.

FIGS. 10a-10e: E-SYT2 promotes NEMO recruitment to the Immune synapse and NF-κB activation in response to TCR-stimulation but no changement in tyrosine phosphorylations and calcium influx. (a) Jurkat cells transfected with either an irrelevant siRNA (Ctrl) or an E-SYT2 directed siRNA were incubated with Raji B cells and prepulsed with SEE. After 10 minutes, cells were fixed and NEMO, CARMA1 and TCR localized by immunofluorescence and confocal microscopy, using anti-NEMO antibody. The corresponding merging of the staining is shown on the right. Scale Bars represent 1 μm. (b) Jurkat cells transfected with either an irrelevant siRNA (Ctrl) or an E-SYT2 directed siRNA or CD4+ T cells purified from parental or E-SYT2 KO mice were stimulated for the indicated periods of time with anti-CD3 and anti-CD28 antibodies. Total lysates were prepared and assayed by immunoblotting with anti-IκBα and anti-phosho-IκBα. (c) IL-2 production of E-SYT2+/+ and E-SYT2−/− lymph nodes T cells stimulated either CD3/CD28 or PMA and Ionomycin. (d) left panel: TCR-mediated tyrosine phosphorylation (time course experiment). Jurkat cells transfected with either an irrelevant siRNA (Ctrl) or an E-SYT2 directed siRNA were stimulated for the indicated periods of time with anti-CD3 and anti-CD28 antibodies. Following lysis in boiling sample buffer, phosphotyrosine-containing proteins were detected by anti-phosphotyrosine immunoblotting. right panel: Jurkat cells transfected with either an irrelevant siRNA (Ctrl) or an E-SYT2 directed siRNA were loaded with Fluo-3 AM and Pluoronic® 1 h at RT. Cells were resuspended in PBS with or without anti-CD3 and anti-CD28 at 4° C. during 30 min and activated either GAM (to crosslink anti-CD3 and anti-CD28 coated-cells) or with PMA/Ionomycin. For fluorescent measurements, a Tecan infinite F500 microplate reader was used at an excitation wavelength of 506 nm and emission of 526 nm, and the cells were maintained at 37° C. for the duration of the measurements. (e) Jurkat cells transfected with either a non-targeting siRNA (NT) or an E-Syt2 directed siRNA were stimulated for the indicated time with anti-CD3 and anti-CD28 antibodies. Total lysates were prepared and assayed by immunoblotting with anti-pERK and anti-ERK, anti-P.p38 or anti-p38 antibodies.

FIGS. 11a-11b: E-Syt2 tethers the ER at the IS but is dispensable for the recruitment of ubiquitinated BCL10 at the ER (A) Jurkat cells transfected with an irrelevant (NT) or E-Syt2-directed siRNA were incubated with superantigen-loaded Raji cells at different times as indicated in order to induce IS formation. Cells were stained for E-Syt2, CARMA1, Calnexin or TCR as indicated and then analyzed by confocal microscopy. (B) Jurkat cells transfected with siRNA were stimulated with anti-CD3 and anti-CD28 antibodies. Crude HM and cytosolic fractions from unstimulated and CD3/CD28 stimulated Jurkat siRNA-depleted cells were analyzed by western blot with the indicated antibodies and the ER resident protein Kinectin.

FIGS. 12a-12d: ESYT2−/− mice develop a skin disease. (a) 6 months and 3 months old mice pictures are shown. (b) Representative histology of wild type and ESYT2 KO mice skin with dermatitis lesions. Tissues from 14 weeks-old mice were fixed, and histological analysis was performed using standard method and hematoxylin and eosin staining (c) Histology and Immunohistochemistry: (a-f) Control mice: (a-b) No histological lesion was detected in the skin of young (>2 month-old) or old (>8 month-old) mice. Heterozygous E-Syt2+/−mice: (c) In young (>2 month-old) mice, no histological lesion was detected in the skin. (d) In old (>6 month-old) mice, lesions were detected, characterized by epidermal hyperplasia and dermal/subcutaneous inflammation. Homozygous E-Syt2−/− mice: (e) lesions in the skin appeared in young (>2 month-old) mice, characterized by epidermal hyperplasia and hyperkeratosis, fibrinous crust formation, and epidermal ulceration. (f) In old (>6 month-old) mice, the lesions were more severe, with a multifocal, complete necrosis and destruction of the epidermis (ulceration), and large fibrinous crust formation. (g-h) E-Syt2˜′˜ mice exhibit an alteration of the T lymphocyte zone organization in the spleen, compared to wild-type mice. In the spleen, lesions were similar in young and old animals. (g) Control mice did not display any alteration of the spleen cell organization (arrows: Periarteriolar Lymphoid Sheaths (PALS) containing T lymphocytes). (h) In contrast, E-Syt2′-′ mice displayed an alteration of the T cell organization. PALS were indeed barely identified, as T cells were diffusely scattered in the spleen. Skin: a-f: HE staining. Spleen: g-h: anti-CD3 immunolabeling. (d) Characterization of the psoriasiform skin lesions in E-Syt2˜′˜ mice. Control mice: (A-B) No histological lesion in young (>2 month-old) and old (>6 month-old) mice. E-Syt2^{+/}˜ mice: (C) No histological lesion in young (>2 month-old) mice. In old (>6 month-old) mice epidermis hyperplasia (D) and dermal infiltration by macrophages (E) were detected. E-Syt2′⁻′ mice: lesions appeared in young (>2 month-old) mice, characterized by (F) epidermal hyperplasia, (G) epidermal ulceration, (H) hyperkeratosis and crust formation, and (I) dermal inflammation sometimes destroying hair follicle (folliculitis). In old (>6 month-old) mice, lesions were more severe, with (J) a multifocal, complete necrosis and destruction of the epidermis (ulceration), and (K) large fibrinous crust formation.

### DETAILED DESCRIPTION

The term “patient” refers to any mammal. The patient may be suffering from an infection due to a gastrointestinal pathogen or may be at risk of developing or transmitting to others an infection due to a gastrointestinal pathogen. In some embodiments the patient is known to have and/or suspected of having a malignancy. In some embodiments the patient is known to have and/or suspected of having a lymphoma. In some embodiments the lymphoma is a MALT-lymphoma or an ABC-DLBCL-lymphoma.

The term “mammal” includes any type of mammal including human, laboratory animals (e.g., primates, rats, mice), livestock (e.g., cows, sheep, goats, pigs, turkeys, chickens), and household companions (e.g., dogs, cats, rodents, etc.).

The term “chemical entity” refers without limitation to any type of agent that may be administered to a cell to modulate the activity of E-SYT2, including without limitation proteins and peptides, nucleic acids, polysaccharides, and any type of organic molecule. According to a particular embodiment, said agent is a nucleic acid or any type of molecule suited for physiological and/or therapeutic administration to a mammal in need thereof, especially a nucleic acid aor an agent as disclosed or defined herein.

### A. E-SYT2

Homo sapiens extended synaptotagmin-like protein 2 (ESYT2 or E-SYT2) (synonyms: FAM62B and CHR2SYT) has the following sequence (SEQ ID NO: 1):

| | |
|---|---|
| 1 | MTPPSRAEAG VRRSRVPSEG RWRGAEPPGI SASTQPASAG RAARHCGAMS GARGEGPEAG |
| 61 | AGGAGGRAAP ENPGGVLSVE LPGLLAQLAR SFALLLPVYA LGYLGLSFSW VLLALALLAW |
| 121 | CRRSRGLKAL RLCRALALLE DEERVVRLGV RACDLPAWVH FPDTERAEWL NKTVKHMWPF |
| 181 | ICQFIEKLFR ETIEPAVRGA NTHLSTFSFT KVDVGQQPLR INGVKVYTEN VDKRQIILDL |
| 241 | QISFVGNCEI DLEIKRYFCR AGVKSIQIHG TMRVILEPLI GDMPLVGALS IFFLRKPLLE |
| 301 | INWTGLTNLL DVPGLNGLSD TIILDIISNY LVLPNRITVP LVSEVQIAQL RFPVPKGVLR |
| 361 | IHFIEAQDLQ GKDTYLKGLV KGKSDPYGII RVGNQIFQSR VIKENLSPKW NEVYEALVYE |
| 421 | HPGQELEIEL FDEDPDKDDF LGSLMIDLIE VEKERLLDEW FTLDEVPKGK LHLRLEWLTL |
| 481 | MPNASNLDKV LTDIKADKDQ ANDGLSSALL ILYLDSARNL PSGKKISSNP NPVVQMSVGH |
| 541 | KAQESKIRYK TNEPVWEENF TFFIHNPKRQ DLEVEVRDEQ HQCSLGNLKV PLSQLLTSED |
| 601 | MTVSQRFQLS NSGPNSTIKM KIALRVLHLE KRERPPDHQH SAQVKRPSVS KEGRKTSIKS |
| 661 | HMSGSPGPGG SNTAPSTPVI GGSDKPGMEE KAQPPEAGPQ GLHDLGRSSS SLLASPGHIS |
| 721 | VKEPTPSIAS DISLPIATQE LRQRLRQLEN GTTLGQSPLG QIQLTIRHSS QRNKLIVVVH |
| 781 | ACRNLIAFSE DGSDPYVRMY LLPDKRRSGR RKTHVSKKTL NPVFDQSFDF SVSLPEVQRR |
| 841 | TLDVAVKNSG GFLSKDKGLL GKVLVALASE ELAKGWTQWY DLTEDGTRPQ AMT |

The human E-SYT2 protein is encoded by the following exemplary E-Syt2 mRNA sequence (SEQ ID NO: 2):

| | |
|---|---|
| 1 | AGTATCCACC CCGCCCGCTC CCGGTGACGT GCCAGCCCCA GGCCCACGCC GCTCCCGCCC |
| 61 | CGCGTGATGA CGCCACCGTC CCGGGCGGAG GCGGGCGTGC GGCGGAGCCG CGTCCCCTCA |
| 121 | GAGGGGCGCT GGCGCGGGGC TGAGCCGCCC GGGATCAGCG CGAGCACCCA GCCCGCCTCG |
| 181 | GCCGGGAGGG CAGCGCGGCA CTGCGGGGCG ATGAGCGGCG CCCGGGGCGA GGGCCCGGAG |
| 241 | GCGGGCGCCG GCGGGGCTGG GGGCCGCGCG GCGCCTGAGA ACCCCGGGGG CGTGCTGAGC |
| 301 | GTGGAGCTGC CCGGGCTGCT GGCGCAGCTG GCGCGGAGCT TCGCGCTGCT GCTGCCCGTG |
| 361 | TACGCGCTGG GCTACCTGGG GCTCAGCTTC AGCTGGGTTC TCCTCGCGCT CGCGCTGCTC |
| 421 | GCCTGGTGTC GCCGCAGCCG CGGCCTCAAG GCCCTGCGCC TGTGCCGCGC GCTGGCGCTG |
| 481 | CTGGAAGACG AGGAGCGCGT CGTGCGCCTG GGGGTGCGCG CCTGCGACCT GCCCGCCTGG |
| 541 | GTTCATTTTC CAGACACTGA AAGAGCAGAA TGGCTAAATA AGACTGTAAA ACACATGTGG |
| 601 | CCTTTCATTT GCCAATTTAT AGAGAAGTTG TTTCGAGAAA CTATAGAACC AGCCGTGCGG |
| 661 | GGAGCAAACA CCCACCTTAG CACCTTTAGT TTCACGAAGG TCGACGTGGG CCAGCAGCCC |
| 721 | CTCAGGATCA ATGGTGTTAA GGTATACACT GAAAATGTAG ACAAAAGGCA AATTATTTTG |
| 781 | GACCTTCAGA TTAGTTTTGT AGGAAATTGT GAGATTGATT TGGAGATCAA ACGATATTTT |
| 841 | TGTAGAGCTG GTGTGAAAAG TATCCAGATT CATGGTACCA TGCGGGTGAT CCTGGAACCG |
| 901 | TTGATTGGAG ATATGCCCTT AGTTGGAGCT TTGTCTATCT TCTTCCTTAG GAAACCACTT |
| 961 | TTAGAAATTA ACTGGACAGG ACTGACGAAT CTTCTGGATG TCCCTGGATT GAATGGTTTA |
| 1021 | TCAGATACTA TCATTTTGGA TATAATATCA AACTATCTGG TGCTTCCCAA TCGAATCACC |
| 1081 | GTTCCACTTG TCAGTGAAGT TCAAATAGCT CAGTTGCGGT TTCCTGTACC AAAGGGTGTT |
| 1141 | CTAAGGATAC ATTTTATTGA AGCTCAGGAT CTTCAGGGGA AAGACACTTA CCTTAAGGGA |
| 1201 | CTTGTCAAGG GAAAGTCAGA CCCCTATGGA ATCATTAGAG TTGGCAACCA AATCTTCCAA |
| 1261 | AGCAGAGTCA TCAAGGAGAA CCTCAGTCCA AAGTGGAATG AAGTCTATGA GGCTTTAGTG |
| 1321 | TATGAACATC CTGGACAAGA ATTAGAGATT GAGCTCTTTG ATGAAGACCC AGACAAGGAT |
| 1381 | GACTTTTTAG GAAGTCTTAT GATTGACCTC ATTGAAGTTG AAAAGGAGCG CCTTTTAGAT |
| 1441 | GAATGGTTCA CTCTGGACGA GGTTCCCAAG GGGAAGCTAC ACTTGAGACT GGAGTGGCTC |
| 1501 | ACGTTAATGC CAAATGCGTC AAACCTCGAC AAGGTGCTAA CAGACATCAA AGCTGACAAA |
| 1561 | GACCAAGCCA ACGATGGTCT TTCCTCTGCA TTGCTGATCT TGTACTTGGA TTCAGCAAGG |
| 1621 | AACCTTCCGT CAGGGAAGAA AATAAGCAGC AACCCAAATC CTGTTGTCCA GATGTCAGTT |
| 1681 | GGGCACAAGG CCCAGGAGAG CAAGATTCGA TACAAAACCA ATGAACCTGT GTGGGAGGAA |
| 1741 | AACTTCACTT TCTTCATTCA CAATCCCAAG CGCCAGGACC TTGAAGTTGA GGTCAGAGAC |
| 1801 | GAGCAGCACC AGTGTTCCCT GGGGAACCTG AAGGTCCCCC TCAGCCAGCT GCTCACCAGT |
| 1861 | GAGGACATGA CTGTGAGCCA GCGCTTCCAG CTCAGTAACT CGGGTCCAAA CAGCACCATC |
| 1921 | AAGATGAAGA TTGCCCTGCG GGTGCTCCAT CTCGAAAAGC GAGAAAGGCC TCCAGACCAC |
| 1981 | CAACACTCAG CTCAAGTCAA ACGTCCCTCT GTGTCCAAAG AGGGGAGGAA AACATCCATC |
| 2041 | AAATCTCATA TGTCTGGGTC TCCAGGCCCT GGTGGCAGCA ACACAGCTCC ATCCACACCA |
| 2101 | GTCATTGGGG GCAGTGATAA GCCTGGTATG GAAGAAAAGG CCCAGCCCCC TGAGGCCGGC |
| 2161 | CCTCAGGGGC TGCACGACCT GGGCAGAAGC TCCTCCAGCC TCCTGGCCTC CCCAGGCCAC |
| 2221 | ATCTCAGTCA AGGAGCCGAC CCCCAGCATC GCCTCGGACA TCTCGCTGCC CATCGCCACC |
| 2281 | CAGGAGCTGC GGCAAAGGCT GAGGCAGCTG GAAAACGGGA CGACCCTGGG ACAGTCTCCA |
| 2341 | CTGGGGCAGA TCCAGCTGAC CATCCGGCAC AGCTCGCAGA GAAACAAGCT TATCGTGGTC |
| 2401 | GTGCATGCCT GCAGAAACCT CATTGCCTTC TCTGAAGACG GCTCTGACCC CTATGTCCGC |
| 2461 | ATGTATTTAT TACCAGACAA GAGGCGGTCA GGAAGGAGGA AAACACACGT GTCAAAGAAA |
| 2521 | ACATTAAATC CAGTGTTTGA TCAAAGCTTT GATTTCAGTG TTTCGTTACC AGAAGTGCAG |
| 2581 | AGGAGAACGC TCGACGTTGC CGTGAAGAAC AGTGGCGGCT TCCTGTCCAA AGACAAAGGG |
| 2641 | CTCCTTGGCA AAGTATTGGT TGCTCTGGCA TCTGAAGAAC TTGCCAAAGG CTGGACCCAG |
| 2701 | TGGTATGACC TCACGGAAGA TGGGACGAGG CCTCAGGCGA TGACATAGCC GCAGCAGGCA |
| 2761 | GGAGGCGTCC TCTTCAGCGT AGCTCTCCAC CTCTACCCGG AACACACCCT CTCACAGACG |
| 2821 | TACCAATGTT ATTTTTATAA TTTCATGGAT TTAGTTATAC ATACCTTAAT AGTTTTATAA |
| 2881 | AATTGTTGAC ATTTCAGGCA AATTTGGCCA ATATTATCAT TGAATTTTCT GTGTTGGATT |
| 2941 | TCCTCTAGGA TTTCGCCAGT TCCTACAACG TGCAGTAGGG CGGCGGTAGC TCTTGTGTCT |
| 3001 | GTGGACTCTG CTCAGCTGTG TCCGTAGGAG TCGGATGTGT CTGTGCTTTA TTATGGCCTT |
| 3061 | GTTTATATAT CACTGAGGTA TACTATGCCA TGTAAATAGA CTATTTTTTA TAATCTTTAC |
| 3121 | ATGCTGGTTT AAATTCAGAA GGAAATAGAT CAAGGAAATA TATATATTTT CTTCTAAAAC |
| 3181 | TTATTAAATT CGTGTGACAA ATAATCATTT TCATCTTGGC AGCAAAAAGT TCTCAGTGAC |
| 3241 | CTATTTTGTG GTGTTTCTTT TTGAAAAGAA AAGCTGAAAT ATTATTAAAT GCTAGTATGT |
| 3301 | TTCTGCCCAT TATGAAAGAT GAAATAAAGT ATTCAAAATA TTAACATTTT CATAAATATA |
| 3361 | AGGATGTATT ATTGAGAAGT AAGTTGAAGG GCTTATAAGG AAAAATGTTT TATAACTGAG |
| 3421 | TAATATATTA AGAGAATTGT CATGGTTCAT AAATCACATT ATGCTAATCT GAAATTTCTT |
| 3481 | ACATAAAAAT GAAGTGTCTT ATGTTTATTT TAATTGCTGT TGTAACTTAC TCATGAAACA |
| 3541 | GTATACAGAC ACCTTGTACT TTTCCTCAAC TGTAAGAGCA GACTTTCAAT GTTAGCAATT |
| 3601 | AAGCTGTTGT CAAACAATCA GTCATGAGCT TTGTTAATTT TCAATGTTTT CCCAGCCTAT |
| 3661 | AAAAAAAGGA AGGTACACGT TGTCCTTTTA AAGGTTGTGA GGTAGATTGG AGTGAGTAGA |
| 3721 | CAGGATATTG CATTAAAAAT TGAAAGCTCG ATCTCATTAT TGTCAGGAAC CCCCAGTGTG |
| 3781 | ACCTCACACA TAGGATGTGG GACCTTTGAG CCGATGTGCA CTGGCCACCA CCAGGGTTGG |
| 3841 | GGGCGCCACA GCTGCGAGCC CGGCCCCTGC TGTTCTCAGC AGCCACTTCC CAGGCTGCCT |
| 3901 | CACTTTATGC CATGACTGAC CTTAATATTG GGATATTGTT ATGCAATTTT TATCCTGTTT |
| 3961 | AGACTGTTAA AAGCAGGTTT CTGTACTTAA GAGTCCCCCA GACCTCCTGT GAGGTGAGGC |
| 4021 | TCTGTTGCAG TGTCGTAGGC TGTGTGTGTC TGTAAAGAAA GAATGCACAT ATGTAGACGA |
| 4081 | TTAAGTGTAT ATTATAAGCT ATATGCTGAA AATGGCTTCC ATAGCCATGA GAACATCTTA |
| 4141 | AAACTATGTG TAAATATATT AAGGAAAGTA TAGCTTTGTA ATTTAAATTG GAGCTTTTAG |
| 4201 | CTTGTTTCAT GGAACTATAC AACTTGTGGG TAACTCACAT GACCAAACAA ACCAAAGTGC |
| 4261 | CTGTGACGGG GCGGGTGGCG TCTACCCACC CTCCCTTCTA GCAGATTCTT ATTTTGTTTG |
| 4321 | AATTTATAAA CAAGGCTGGT GGCTGTCTAC CCACCCTCCC TTCTAGCAGA TTTTGATTTT |
| 4381 | GTTTCAATTT ATAACTTACA CTTTGAACCA TGGGTTTACT TATAATGGAG TCTGTAGCTT |
| 4441 | CACAGCATAT TTCATGTAAT CATAAAGACC AGTATATTCC CTCCTGCTGA ATGACATGCG |
| 4501 | ACTGTAAAGC CTCTTTATAA ACCATTTCCA ATGTTAGTAT ATAGGATTAT TTGGGAAGCG |
| 4561 | TATCAATACC TTTATAGACA AATACGAACA TGTATGCACA CAAAACATTT AACTATGGTA |
| 4621 | TTTATGGAAG ACAGGTAACA ACATTAAATC TAGTTGCTTT CCCTTAGTAT TAGATTTGTT |
| 4681 | GAGGGTTTTT TAAAAATCAG GTCTGTTGAA AGTCTTCTGT CATAATCTAT AAAGCAGCAG |
| 4741 | CACTCATGGA AATTGTAGCA TGCCAGTAAT TTTTACCAAC ATCCCATACA TCTGAGTTCT |
| 4801 | GCAGTCCAGT GTGTAATCCG CTCCATGTGT ATTTTGCTTA ATGGAATGCT TTATTTAAGC |
| 4861 | ACTTAGGCAG AGTAGACACA ATTAAAGGTA CAAAGCCCAG AGGAAGTGGT AGAGCAGCAC |
| 4921 | CGTGCCTGCC CTGAGGCAGT GGAGTCAGTA GCGCTGTCCC CAGGGCCTTG AGTGCCTGGA |
| 4981 | GGTGCTTGGC CTCCAGTAGC TGCCTCCATT CTCTTTTAAA AAAAGGGGGT GATTCTGAGG |
| 5041 | CACTGAAGTG CCTCCCAGAT GTGGAGGAGT GAAGCCACCA TCGAGGCCAC ACTCAGCACT |
| 5101 | CCAGGATCCC AGCGATGTCA GACACTCTTG AGTTGTCAAA ACGTTAATTT TCAGTTTTAA |
| 5161 | ATAATCAGTT TATCTAAGAA AAGGGAATTT TAACTTTTCT ACCTTGAGCC AAGCCAATGA |
| 5221 | AGGGAAAATT AATTAACTTA GTAAATTTGA AGTGCAGCTC TGTTAGCTCG TACATGTGGG |
| 5281 | TTCTTATCCT GATCCTGTGC CTTAAAGTAG GAAGGTGTTT CCAAGTTCAG ATTAAAATAG |
| 5341 | AAGCAGCTGG CCGGGTGCGG TGGCTCACGC CTGTAATCCC AGCACTTTGG GAGGCCGAGG |
| 5401 | CGGGCGGATG ACCTGAGGTC AGGAGTTCAA GACCAGCCTG GCCAACATGG AGAAACCCCA |
| 5461 | TCTTTACTAA AAATACAAAA ATTAGCCGGG CGTGGTGGTG GAGCGCACGC CTGTAGTCCC |
| 5521 | AGCTACTCGG GAGGCTGAGG CAGGAGAATT GCTTGAACCC AGGAGGCGGA GGTTGCAGTG |
| 5581 | AGCCAAGATC GCACCACTGC ACTCCAGCCT GGGCAACAGA GTGAGACTCC GTCTCAAAAA |
| 5641 | ATAAATAAAA TAGAAGCAGC CTTGTAACTG TATTTACCAT GATAATATAT TCTGCACGGT |
| 5701 | AAGAATTCCT TTTACAGACA TTCTTTATCA AGAGGTCGGC CCTTCTTTTT CAGGCACATA |
| 5761 | AGCCAAATGC AGGCCTGTGT GTAGCTGTGT GTTTTTTCTG TGGTTGCCGC ATTTATTCCA |
| 5821 | CCTCCAGCTG GACCCCCCAC TGCAAATAGA GAACAGCGGT GGGGGATGGG GGTTAAAAAG |
| 5881 | TAGAGAACCT CCTTTCTGTT CAACTAATTT CACGTGACAG TGCATGTATT TATTCAATAA |
| 5941 | AACCTTTATG TTAGCTC |

E-Syt2 homologues are known in other mammals, including mouse, and skilled artisans are able to use the relevant E-Syt2 sequence to practice embodiments of the invention in non-human mammals.

### B. Methods of Identifying E-SYT2 Modulators

This invention provides methods of identifying an E-SYT2 modulator. The E-SYT2 modulator may be an E-SYT2 inhibitor or an E-SYT2 inducer. An E-SYT2 inhibitor is a chemical entity that reduces the activity of E-SYT2 relative to what the E-SYT2 activity level would be in the absence of the chemical entity. An E-SYT2 inducer is a chemical entity that increases the activity of E-SYT2 relative to what the E-SYT2 activity level would be in the absence of the chemical entity.

The methods may comprise providing a cell that expresses E-SYT2; contacting the cell with a candidate chemical entity; and measuring at least one feature in the cell mediated by E-SYT2. In some embodiments the at least one feature is also measured in the absence of the candidate chemical entity and the feature in the presence of the chemical entity is compared to the feature in the absence of the chemical entity. In some embodiments the cell is a T cell. In some embodiments the cell is a B cell. In some embodiments the at least one feature is selected from recruitment of CARMA1, BCL10, NEMO, and PKCθ to the immunological synapse (IS). In some embodiments the rate and/or the extent of recruitment of at least one of CARMA1, BCL10, NEMO, and PKCθ to the IS is reduced in the presence of the candidate chemical entity, and the candidate chemical entity is thereby identified as an inhibitor of E-SYT2 activity. In some embodiments the rate and/or the extent of recruitment of at least one of CARMA1, BCL10, NEMO, and PKCθ to the IS is increased in the presence of the candidate chemical entity, and the candidate chemical entity is thereby identified as an inducer of E-SYT2 activity.

In some embodiments the chemical entity binds to E-SYT2 protein to modulate E-SYT2 protein function. In some embodiments the chemical entity binds to a non-E-SYT2 protein in a cell to modulate the interaction of that protein with E-SYT2, to thereby modulate E-SYT2 protein activity in the cell.

In some embodiments the chemical entity modulates expression of E-Syt2 to thereby modulate the level of E-SYT2 protein in the cell. If the modulation in expression of E-SYT2 protein in the cell causes an increase or decrease in E-SYT2 activity in the cell then the chemical entity is an inducer or inhibitor of E-SYT2 activity.

### C. E-SYT2 Modulators

This invention provides E-SYT2 modulators. In some embodiments the E-SYT2 modulator is a chemical entity that modulates E-SYT2 activity in an assay disclosed in Section B of this detailed description section.

The E-SYT2 modulator may be an E-SYT2 inhibitor or an E-SYT2 inducer. An E-SYT2 inhibitor is a chemical entity that reduces the activity of E-SYT2 relative to what the E-SYT2 activity level would be in the absence of the chemical entity. An E-SYT2 inducer is a chemical entity that increases the activity of E-SYT2 relative to what the E-SYT2 activity level would be in the absence of the chemical entity.

In some embodiments the chemical entity binds to E-SYT2 protein to modulate E-SYT2 protein function. In some embodiments the chemical entity binds to a non-E-SYT2 protein in a cell to modulate the interaction of that protein with E-SYT2, to thereby modulate E-SYT2 protein activity in the cell.

In some embodiments the chemical entity modulates expression of E-Syt2 to thereby modulate the level of E-SYT2 protein in the cell. If the modulation in expression of E-SYT2 protein in the cell causes an increase or decrease in E-SYT2 activity in the cell then the chemical entity is an inducer or inhibitor of E-SYT2 activity.

### E. Methods of Inhibiting NF-κB Activity

The data reported in the examples demonstrates that an E-SYT2 modulator may be used to modulate the activity of E-SYT2 in a cell to result in modulation of NF-κB activity in the cell. Accordingly, this invention also provides methods of modulating NE-KB activity in a cell comprising contacting the cell with an E-SYT2 modulator. In some embodiments the NE-KB activity in the cell is inhibited by a method comprising the treatment of the cell with an E-SYT2 inhibitor. In some embodiments the NE-KB activity in the cell is induced by a method that comprise contacting the cell with an E-SYT2 inducer. In some embodiments contacting the cell with the E-SYT2 modulator impairs recruitment of at least one of CARMA1, BCL10, NEMO, PKCθ, and CD69 to the immunological synapse (IS) in the cell. In some embodiments contacting the cell with the E-SYT2 inhibitor reduces recruitment of at least one of CARMA1, BCL10, NEMO, and PKCθ to the immunological synapse (IS) in the cell. In some embodiments the E-SYT2 inhibitor binds to E-SYT2 protein to modulate E-SYT2 protein function. In some embodiments the E-SYT2 modulator regulates expression of E-SYT2. In some embodiments the cell in cultured in vitro. In some embodiments the cell is in a patient and the method comprises administering the E-SYT2 modulator to the patient. In some embodiments NF-κB is constitutively active in the cell in the absence of an E-SYT2 modulator. In some embodiments the cell is a T cell. In some embodiments the cell is a B cell.

The invention is also directed to a E-SYT2 modulator, especially a E-SYT2 inhibitor, as disclosed or defined herein, for use for treating lymphoma or its symptoms, in particular a MALT-lymphoma or a ABC-DLBCL-lymphoma, especially when these diseases or symptoms are associated with increased NF-κB activity. According to a particular embodiment, the said E-SYT2 modulator further inhibits NF-κB activity in lymphoma cells and/or reduces recruitment of at least one of CARMA1, BCL10, NEMO, and PKCθ to the immunological synapse (IS) in said cells.

The terms “treating” or “treatment” as used herein mean curing, reversing, attenuating, alleviating, minimizing, suppressing or halting the effects of the condition or disease for which a treatment is sought. These terms are not restricted to removing the cause(s) of the disease. Therefore, these expressions encompass the curative effect achieved with agents of the invention and also the beneficial effect for an animal or patient undergoing the treatment, said effect being either obtained at cellular level or clinical level, including as a result, an improvement of the condition of the animal or patient and/or a remission state or a recovery of a health state.

According to a particular embodiment, the E-SYT2 modulator, as disclosed or defined herein, for use for treating lymphoma, or its symptoms, is a E-SYT2 inhibitor as disclosed or defined herein, which binds to E-SYT2 protein to inhibit E-SYT2 protein function, or is a E-SYT2 inhibitor which inhibits expression of E-Syt2.

According to a particular embodiment, said lymphoma or symptom(s) thereof is(are) associated with increased NF-κB activity. Accordingly, the E-SYT2 modulator may further inhibits NF-κB activity in lymphoma cells and/or reduces recruitment of at least one of CARMA1, BCL10, NEMO, and PKCθ to the immunological synapse (IS) in said cells.

### F. Methods of Characterizing Lymphoma

Since alterations of the CARMA1, BCL10, and MALT1 genes have been correlated with the development of MALT-lymphoma or a ABC-DLBCL-lymphoma, the data in the Examples suggest that gain of function mutations of E-Syt2 are present in at least some of these malignancies. Accordingly, this invention also provides methods providing a sample from a patient suspected of having or at risk of having lymphoma; and screening the sample to identify the presence and/or absence of a gain of function E-Syt2 mutation in the sample. The presence of a gain of function E-Syt2 mutation in the sample indicates that the patient has, is at risk of having, and/or is at risk of developing lymphoma. In some embodiments the lymphoma is selected from a MALT-lymphoma and a DLBCL-lymphoma (e.g., an ABC-DLBCL-lymphoma).

MALT lymphoma is a form of lymphoma involving the mucosa-associated lymphoid tissue (MALT), frequently of the stomach, but virtually any mucosal site can be afflicted. It is a cancer originating from B cells in the marginal zone of the MALT, and is also called extranodal marginal zone B cell lymphoma.

Diffuse large B-cell lymphoma (DLBCL or DLBL), a cancer of B cells, is the most common type of non-Hodgkin lymphoma among adults, with an annual incidence of 7-8 cases per 100,000 people per year. This cancer occurs primarily in older individuals, with a median age of diagnosis at approximately 70 years of age, though it can also occur in children and young adults in rare cases. DLBCL is an aggressive tumor which can arise in virtually any part of the body, and the first sign of this illness is typically the observation of a rapidly growing mass, sometimes associated with fever, weight loss, and night sweats.

Diffuse large B-cell lymphoma encompasses a biologically and clinically diverse set of diseases, many of which cannot be separated from one another by well-defined and widely accepted criteria. Gene expression profiling studies have also attempted to distinguish heterogeneous groups of DLBCL from each other. These studies examine thousands of genes simultaneously using a DNA microarray, looking for patterns, which may help in grouping cases of DLBCL. Many studies now suggest that cases of DLBCL, NOS can be separated into two groups on the basis of their gene expression profiles; these groups are known as germinal center B-cell-like (GCB) and activated B-cell-like (ABC). Tumor cells in the germinal center B-cell-like subgroup resemble normal B cells in the germinal center closely, and are generally associated with a favorable prognosis. Activated B-cell-like tumor cells are associated with a poorer prognosis, and derive their name from studies, which show the continuous activation of certain pathways normally activated when B cells interact with an antigen. The NF-κB pathway, which is normally involved in transforming B cells into plasma cells, is an important example of one such pathway.

In some embodiments, if the sample comprises a gain of function E-SYT2 mutation the patient is diagnosed as having a MALT-lymphoma or a ABC-DLBCL-lymphoma. In some embodiments, if the sample comprises a gain of function E-SYT2 mutation the patient is diagnosed as having an increased risk of having and/or developing a MALT-lymphoma or a ABC-DLBCL-lymphoma.

In some embodiments the screening comprises analysing a nucleic acid present in the sample. In some embodiments the screening comprises performing a hybridization and/or polymerization assay on the nucleic acid.

If genomic DNA is analyzed, the tissue sample can come from any tissue source that comprises genomic DNA of the subject, including, without limitation, synovial fluid, blood, blood-derived product (such as buffy coat, serum, and plasma), lymph, urine, tear, saliva, hair bulb cells, cerebrospinal fluid, buccal swabs, feces, synovial fluid, synovial cells, sputum, or tissue samples. In addition, one of skill in the art would realize that some samples would be more readily analyzed following a fractionation or purification procedure, for example, isolation of DNA from whole blood. If mRNA in the sample is analyzed, the tissue sample will generally be taken from a tissue of the subject in which E-Syt2 is known to be expressed. In some embodiments the sample comprises lymphocytes.

In some embodiments the test sample is collected from the subject and then tested with little or no sample processing. In some embodiments the sample is processed, such as for example and without limitation processing to isolate all or a portion of the nucleic acid in the sample, such as genomic DNA in the sample, total RNA in the sample, or mRNA in the sample.

In general, methods for detecting an allele at a SNP locus can be divided into two groups: (1) methods based on hybridization analysis of polynucleotides, and (2) other methods based on biochemical detection or sequencing of polynucleotides. The method used may be based on analysis of a starting nucleic acid that is total RNA or mRNA obtained from the subject. In some embodiments cDNA is made from the mRNA as part of the method. Alternatively, the method used may be based on analysis of a starting nucleic acid that is genomic DNA obtained from the subject.

Any method known in the art or later developed may be used, in view of the teachings of this disclosure, to detect a gain of function mutation in a starting sample that is genomic DNA obtained from a subject. Exemplary methods include, by way of example only, large-scale SNP genotyping, exonuclease-resistant nucleotide detection, solution-based methods, genetic bit analyses, primer-guided nucleotide incorporation, allele specific hybridization, and other techniques. Any method of detecting a marker may use a labeled oligonucleotide.

Numerous methods and devices are well known to the skilled artisan to identify the presence or absence of a mutant allele. DNA (genomic and cDNA) for SNP detection can be prepared from a biological sample by methods well known in the art, e.g., phenol/chloroform extraction, PURE GENE DNA® purification system from GentAS Systems (Qiagen, CA). Detection of a DNA sequence may include examining the nucleotide(s) located at either the sense or the anti-sense strand within that region. The presence or absence of a SNP allele in a patient may be detected from DNA (genomic or cDNA) obtained from PCR using sequence-specific probes, e.g., hydrolysis probes from Taqman, Beacons, Scorpions, or hybridization probes that detect an allele. For the detection of the allele, sequence specific probes may be designed such that they specifically hybridize to the genomic DNA for the alleles of interest or, in some cases, an RNA of interest. For example, primers and probes for a mutant allele may be designed based on context sequences found in the NCBI SNP database available at: www.ncbi.nlm.nih.gov/snp. These probes may be labeled for direct detection or contacted by a second, detectable molecule that specifically binds to the probe. The PCR products also can be detected by DNA-binding agents. Said PCR products can then be subsequently sequenced by any DNA sequencing method available in the art. Alternatively the presence or absence of an allele can be detected by sequencing using any sequencing methods such as, but not limited to, Sanger-based sequencing, pyrosequencing or next generation sequencing (Shendure J. and Ji, H., Nature Biotechnology (1998), Vol. 26, Nr 10, pages 1135-1145). Optimised allelic discrimination assays for SNPs may be purchased from Applied Biosystems (Foster City, Calif., USA).

Various well-known techniques can be applied to interrogate a particular mutation, including, e.g., hybridization-based methods, such as dynamic allele-specific hybridization (DASH) genotyping, SNP detection through molecular beacons (Abravaya K., et al. (2003) Clin Chem Lab Med. 41:468-474), Luminex xMAP technology, Illumina Golden Gate technology and commercially available high-density oligonucleotide SNP arrays (e.g., the Affymetrix Human SNP 5.0 GeneChip performs a genome-wide assay that can genotype over 500,000 human SNPs), BeadChip kits from Illumina, e.g, Human660W-Quad and Human 1.2M-Duo); enzyme-based methods, such as restriction fragment length polymorphism (RFLP), PCR-based methods (e.g., Tetra-primer ARMS-PCR), Invader assays (Olivier M. (2005) Mutat Res. 573(1-2): 103-10), various primer extension assays (incorporated into detection formats, e.g., MALDI-TOF Mass spectrometry, electrophoresis, blotting, and ELISA-like methods), Taqman assays, and oligonucleotide ligase assays; and other post-amplification methods, e.g., analysis of single strand conformation polymorphism (Costabile et al. (2006) Hum. Mutat. 27(12): 1163-73), temperature gradient gel electrophoresis (TGGE), denaturing high performance liquid chromatography, high-resolution melting analysis, DNA mismatch-binding protein assays (e.g., MutS protein from Thermus aquaticus binds different single nucleotide mismatches with different affinities and can be used in capillary electrophoresis to differentiate all six sets of mismatches), SNPLex® (proprietary SNP detecting system available from Applied Biosystems), capillary electrophoresis, mass spectrometry, and various sequencing methods, e.g., pyrosequencing and next generation sequencing, etc. Commercial kits for SNP genotyping include, e.g., Fluidigm Dynamic Array® IFCs (Fluidigm), TaqMan® SNP Genotyping Assay (Applied Biosystems), MassARRAY® iPLEX Gold (Sequenom), and Type-it Fast® SNP Probe PCR Kit (Quiagen).

In some embodiments, the presence or absence of an allele in a patient is detected using a hybridization assay. In a hybridization assay, the presence or absence of the genetic marker is determined based on the ability of the nucleic acid from the sample to hybridize to a complementary nucleic acid molecule, e.g., an oligonucleotide probe. A variety of hybridization assays are available. In some, hybridization of a probe to the sequence of interest is detected directly by visualizing a bound probe, e.g., a Northern or Southern assay. In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide or binding agent (e.g., SYBR® Green), is allowed to contact the membrane under low-, medium- or high-stringency conditions. Unbound probe is removed and the presence of binding is detected by visualizing the labeled probe. In some embodiments, arrays, e.g., the MassARRAY system (Sequenom, San Diego, Calif., USA) may be used to genotype a subject.

Sequence-Specific Oligonucleotide (SSO) typing uses PCR target amplification, hybridization of PCR products to a panel of immobilized sequence-specific oligonucleotides on beads, detection of probe-bound amplified product by color formation followed by data analysis. Those skilled in the art would understand that the described Sequence-Specific Oligonucleotide (SSO) hybridization may be performed using various commercially available kits, such as those provided by One Lambda, Inc. (Canoga Park, Calif.) coupled with Luminex® technology (Luminex, Corporation, TX). LABType® SSO is a reverse SSO (rSSO) DNA typing solution that uses sequence-specific oligonucleotide (SSO) probes and color-coded microspheres to identify alleles. The target DNA is amplified by polymerase chain reactions (PCR) and then hybridized with the bead probe array. The assay takes place in a single well of a 96-well PCR plate; thus, 96 samples can be processed at one time.

Sequence Specific Primers (SSP) typing is a PCR based technique which uses sequence specific primers for DNA based allele typing. The SSP method is based on the principle that only primers with completely matched sequences to the target sequences result in amplified products under controlled PCR conditions. Allele sequence-specific primer pairs are designed to selectively amplify target sequences, which are specific to a single allele or group of alleles. PCR products can be visualized on an agarose gel. Control primer pairs that matches non-allelic sequences present in all samples act as an internal PCR control to verify the efficiency of the PCR amplification. Those skilled in the art would understand that low, medium and high resolution genotyping with the described sequence-specific primer typing may be performed using various commercially available kits, such as the Olerup SSP™ kits (Olerup, Pa.) or (Invitrogen) or Allset And™Gold DQA1 Low resolution SSP (Invitrogen).

Sequence Based Typing (SBT) is based on PCR target amplification, followed by sequencing of the PCR products and data analysis. In some cases, RNA, e.g., mature mRNA or pre-mRNA, can also be used to determine the presence or absence of alleles. Analysis of the sequence of mRNA transcribed from a given gene can be performed using any known method in the art including, but not limited, to Northern blot analysis, nuclease protection assays (NPA), in situ hybridization, reverse transcription-polymerase chain reaction (RT-PCR), RT-PCR ELISA, TaqMan-based quantitative RT-PCR (probe-based quantitative RT-PCR) and SYBR green-based quantitative RT-PCR. In one example, detection of mRNA levels involves contacting the isolated mRNA with an oligonucleotide that can hybridize to mRNA encoded by a coding sequence comprising a gain of function allele. The nucleic acid probe can typically be, for example, a full-length cDNA, or a portion thereof, such as an oligonucleotide of at least 7, 15, 30, 50, or 100 nucleotides in length and sufficient to specifically hybridize under stringent conditions to the mRNA. Hybridization of an mRNA with the probe indicates that the marker in question is being expressed. In one format, the RNA is immobilized on a solid surface and contacted with a probe, for example by running the isolated RNA on an agarose gel and transferring the mRNA from the gel to a membrane, such as nitrocellulose. Amplification primers are defined as being a pair of nucleic acid molecules that can anneal to 5′ or 3′ regions of a gene (plus and minus strands, respectively, or vice-versa) and contain a short region in between. In general, amplification primers are from about 10 to about 30 nucleotides in length and flank a region from about 50 to about 200 nucleotides in length. Under appropriate conditions and with appropriate reagents, such primers permit the amplification of a nucleic acid molecule comprising the nucleotide sequence flanked by the primers. PCR products can be detected by any suitable method including, but not limited to, gel electrophoresis and staining with a DNA-specific stain or hybridization to a labeled probe.

In some embodiments, the presence of a gain of function allele in a subject is determined by measuring RNA levels using, e.g., a PCR-based assay or reverse-transcriptase PCR (RT-PCR). In some embodiments, quantitative RT-PCR with standardized mixtures of competitive templates can be utilized.

A gain of function allele can also be identified by detecting an equivalent genetic marker thereof, which can be, e.g., a SNP allele on the same haplotype as the gain of function allele. Two particular alleles at different loci on the same chromosome are said to be in linkage disequilibrium (LD) if the presence of one of the alleles at one locus tends to predict the presence of the other allele at the other locus. The SNP may be an allele of a polymorphism that is currently known. Other SNPs may be readily identified by the skilled artisan using any technique well-known in the art for discovering polymorphisms.

In some embodiments the screening comprises analysing the activity of E-SYT2 protein in the sample. In some embodiments the activity of E-SYT2 protein is analyzed by a method that comprises recruitment of Carma1, Bcl10, NEMO, and PKCθ to the immunological synapse (IS).

### G. Genetically Modified Mammals

In another aspect, the invention provides genetically modified mammals comprising one or two mutant alleles of E-Syt2. In some embodiments the mutant alleles are gain of function alleles. In some embodiments the mutant alleles are loss of function alleles. In some embodiments the mutant alleles are loss of function alleles and the genetically modified mammals develop dermatitis. In some embodiments the genetically modified mammal is a non-human mammal. In some embodiments the genetically modified mammal is a mouse.

In some embodiments the animal is heterozygous for at least one E-Syt2 mutant allele. In some embodiments the animal is homozygous for E-Syt2 mutant allele. In some embodiments the genetically modified mammal comprises at least one conditional mutant E-Syt2 allele, which may be a conditional allele that has been recombined to delete a portion of the E-Syt2 gene or an allele that has not been so recombined but is capable of such recombination.

In some embodiments the genetically modified animal comprises one mutant allele of E-Syt2 that encodes a mutant E-SYT2 protein, wherein the mutant allele when homozygous confers on a homozygous animal a mutant phenotype that is not present in the heterozygous mammal. In some embodiments the two mutant alleles of E-Syt2 are the same allele while in other embodiments the alleles are different, meaning that the alleles comprise different nucleotide sequences and/or comprise different regulatory sequences that cause a difference in their expression

The invention also provides genetically modified mammals comprising two copies of a conditional loss of function allele of E-Syt2, wherein the conditional allele is recombined in the thymus of the genetically modified mammal such that the thymus of the mammal comprises cells that do not express functional E-SYT.

### Examples

### Materials and Methods

Cell Cultures, Antibodies and Transfections.

Jurkat cell clones J77c120, CARMA1-deficient JPM50.6 cells and the APC Raji B cells were grown in complete RPMI medium containing 10% (vol/vol) fetal bovine serum, nonessential amino acids, and 1-glutamine (Das et al., 2004) (Gibco, life technologies). HEK293T cells, derived from human embryonic kidney cells (HEK293) and stably expressing the SV40 large T antigen, were grown in complete DMEM supplemented with FBS (10%), nonessential amino acids, and 1-glutamine (Gibco, life technologies).

Plasmids, siRNA and Antibodies

E-Syt2 cDNA was cloned in pEGFP.C2 plasmid (clontech) in order to obtain GFP-E-Syt2, a gift from Dr. T. Moss (CRCHU, Quebec, Canada). pCMV5-myc-ESyt2 was a kind gift of Dr. Thomas Sudhof (Stanford University, USA). pCDNA3-HA-ESyt2 was obtained by cloning E-Syt2 into the pCDNA3-HA vector. pCR3-VSV-CARMA1 WT and pCR3-VSV-CARMA1 ASH3 were gifts from Dr. Margot Thome Miazza (University of Lausanne, Switzerland). pCR3-VSV-CARMA1-L808P was generated by site directed mutagenesis with a pCR-based strategy. PKC6 WT, A148E (CA) or K409R (DN) cloned into pEFneo His/Xpress (Invitrogen) were kind gifts from Dr. Amnon Altman (La Jolla Institute, USA). Human SMARTpool ON-TARGETplus ESYT2 (L-025231-01), SMARTpool ON-TARGETplus Esyt1(L-043501-01) and ON-TARGETplus Non-targeting Pool siRNAs (D-001810-10) were purchased from Dharmacon. The following antibodies were used: anti-VSV mAb (P5D4), anti-Myc mAb (9E10), anti-HA.11 mAb (16B12, ref MMS-101P, Covance), anti-E-Syt1 rabbit polyclonal antibody is a gift of Dr. Pietro De Camilli (Yale University, USA), anti-E-Syt2 rabbit polyclonal antibody was generated against a TrpE fusion protein encompassing the last 200 amino acids of the C-terminal part of human E-Syt2, anti-PKCθ mAb (Clone 27, BD), anti-α Tubulin mAb (DM1A; Sigma), anti-NEMO mAb (No. 611306, BD), anti-BCL10 mAb (4A8; Origene), anti-CARMA1 rabbit polyclonal antibody (ab91463; Abcam), anti-TCR mAb (santa cruz), anti-LCK mAb (3A5; Santa cruz), anti-ADAP mAb (No. 610345; BD), anti-phosphoTyrosine (4G10; Millipore), anti-CD3 (UCHT1; BioLegend, MEM92; Exbio) anti-CD28 (CD28.2; Sigma), anti-calnexin (AF18, Pierce), anti-Kinectin (H-190, sc-33562, santa cruz), anti-BCL10 (A-6, sc-13153; santa cruz), anti-p-IKBa (5A5, No. 9246; CST), anti-p-IKBa mAb (14D4; CST), rabbit anti-p65/RelA (raised against the peptide sequence ADMDFSALLSQISS; provided by N. Rice, National Cancer Institute Frederick Cancer Research and Development Center, Frederick, Md.), Anti-ERK mAb (sc-1647; santa cruz) anti-pERK (12D4; Santa cruz), anti-P38 (sc535; Santa cruz), anti pP38 (sc7973; santa cruz), anti-pIKK (16A6; CST).

Antibodies Used for Flow Cytometric Analysis:

Conjugated anti-CD3, anti-CD19, anti-CD62L, anti-CD8, anti-CD4, anti-CD44, anti-CD25 (BD Bioscience), anti-annexinV (BD pharmingen), anti-FoxP3 (eBioscience). Secondary antibodies for immunofluorescence were supplied by Molecular Probes (Alexa Fluor conjugates).

Transfections

DNA constructs were inserted (electroporated) into Jurkat cells using the Invitrogen Neon Transfection system 82. Transiently transfected cells were imaged 24-48 h after transfection. Transfection efficiency was evaluated by western blot. HEK-293T cells were transfected with the Calcium Phosphate transfection method. Transient transfection of Jurkat cells with siRNAs was performed by electroporation using a gene pulser II (BioRad).

Cell Extracts, Immunoprecipitation and Immunoblotting

Lysis, immunoprecipitation and immunoblotting were performed as previously described.⁸³

Primary CD4 T Cell Culture and Transfections.

Peripheral blood cells were grown in the presence of staphylococcal enterotoxin B (SEB; 5 ng/ml) for 48-72 h, washed, and cultured in IL-2-containing medium (20-50 U/ml) for 7 d, at which time they were restimulated with SEB (5 ng/ml) and PHA (0.4 μg/ml) for 24 h. Cells were resuspended for another 24 h in IL-2-containing medium, and then negatively selected through magnetic bead purification (MACS; Miltenyi Biotec) according to the manufacturer's instructions. CD4 T cells were cultured for 5-6 d in complete RPMI medium enriched with 20 U/ml of human IL-2. DNA constructs were electroporated into primary CD4 T cells using the Invitrogen Neon Transfection system (PBMC program) 82. Primary CD4 T cells were imaged 24-48 h after electroporation. Transfection efficiency was evaluated by western blot.

Cell Stimulation for Confocal Microscopy

Immunological synapse formation was promoted by incubating Raji cells unpulsed or pulsed with superantigen (10 μg/ml, 20 min), either SEE for Jurkat or SEB for primary CD4 T cells at a 1:1 T cell/APC ratio for 30 min. For synaptic clusters imaging, glass coverslips were washed 2-3 times in optical grade acetone and soaked overnight in 0.1 M KOH. Coverslips were then thoroughly rinsed in deionized water and dried. The glass cover-slips were coated overnight at room temperature with 0.001% poly-L-Lysine (Sigma-Aldrich) diluted in PBS. Dried coverslips were subsequently incubated with stimulatory antiCD3 mAb (MEM 92; EXBIO), antiCD28 mAb (Beckman coulter France 1M1376) and with a combination of the two, or nonstimulatory antiCD45 mAb (GAP 8.3; ATCC) antibodies at concentration of 10 μg/ml overnight at 4° C.⁸². Cells were resuspended in imaging buffer⁸⁴, and 500,000 to 1.10⁶ cells were dropped onto the coverslips and incubated for 5 min at 37° C.

Confocal Imaging

Cells plated onto poly-L-lysine-coated coverslips were fixed in 4% paraformaldehyde for 15 min, rinsed, and treated with 50 mM NH4Cl in PBS for 10 min to quench the aldehyde groups. After PBS wash, nonspecific binding was prevented by 15-min incubation with 1% BSA (wt/vol) and 0.05% saponin in PBS, used throughout the procedure as staining and washing buffer. After 1-h incubation with the indicated primary antibodies, cells were rinsed and incubated with the corresponding secondary antibodies. The anti-Lck mAb (3A5) and the anti-TCRz mAb were obtained from Santa Cruz Biotechnology, Inc.; the anti-CARMA1 (AB91463) and the anti-PCKO (ab110728) were purchased from ABCAM LTD, the anti-pPKC was purchased from Biosource (MBS000420). The cyanine 3 (Cy3)-coupled anti-mouse IgG2b, the fluorescein-coupled anti-mouse IgG2b, the fluorescein-coupled anti-mouse IgG2a and the fluorescein-coupled goat anti-rabbit Ig were from Jackson ImmunoResearch Laboratories. Alexa Fluor 488-coupled goat antifluorescein antibody was obtained from Molecular Probes. Confocal images were obtained using a LSM 700 confocal microscope (Carl Zeiss) over a 63× objective. Z stack optical sections were acquired at 0.2 μm depth increments, and both green and red laser excitation were intercalated to minimize cross-talk between the acquired fluorescence channels.

Confocal Image Post-Treatment

Complete image stack deconvolution was performed with Huygens Essential (version 3.0, Scientific Volume Imaging), and 2D images were generated from a maximum intensity projection over a 3D volume cut of 1 μm depth, centered either on the vesicular compartment when visible or on the cell center.

Cluster Segmentation in Confocal Images

CD4⁺ T cells were loaded on a CD3 or CD28-coated coverslips for 5 min. Signaling clusters were stained for the proteins of interest. Confocal images were acquired at 0.2 μm increments in the Z-axis and only the optical slice closer to the coverslip was analyzed. Cluster analysis was carried through the ImageJ based Fiji software. Briefly, supervised machine learning Weka algorithm was manually trained to both segment individual clusters in imaged T-cells and the cell frame-area in the single z-frame. The segmentation features extracted by Weka were then applied to the range of acquired datasets. The Particle Analyser (ImageJ API) was then applied to extract the area and intensity of each individual cluster and the frame-area of the cells. Resulting information was then used to calculate for each individual cell: the cluster density (clusters area per cell area) and the average cluster intensity per mm² (the integrated clusters intensity per cell area).

Mice

To address whether E-Syt2 is required for the development and activation of thymocytes, we generated mice carrying a conditional E-Syt2 knockout allele in the thymus using the Cre-lox system. A gene-targeting vector, in which two LoxP sites were inserted into some introns flanking exons of E-Syt2 (especially introns flanking exons 17-18 of E-Syt2), was constructed (EUCOMM-KOMP CSD). The targeting vector also included a LacZ-neo cassette flanked by a third loxP site. In this way, existence of Cre recombinase would lead to deletion of the exons (especially exons 17 and 18) and subsequent inactivation of E-Syt2. Following electroporation of linearized targeting vector into embryonic stem (ES) cells from the C57BL/6N strain, neomycin-resistant colonies were screened for homologous recombinants. E-Syt2^{FLN/+} ES cells (FLN=flox-neo-lacZ) were injected into blastocysts from the BALB/cN strain and mice with germline transmission of the E-Syt2 allele were obtained. E-Syt2^{flox-neo/+} heterozygous mice were intercrossed to generate E-Syt2^{flox-neo/flox-neo} mice. These homozygous mutant mice had no detectable E-Syt2 protein expression (and was referred as E-Syt2−/− mice) while the expression of E-Syt2 was half-reduced in heterozygous mice. This indicates that the presence of neomycine gene (neo) blocked the normal splicing of E-Syt2 mRNA as previously described for other genes (1, 2) and that homozygous E-Syt2^{FLN/FLN} mice showed phenotypes similar to those of E-Syt2-null mice and were thereafter called E-Syt2′⁻′ mice. The Frt site-flanked drug selection cassette (lacZ-neo) was remove by crossing E-Syt2^{flox-neo/flox-neo} mice with transgenic Flp mice expressing Flp recombinase to obtain E-Syt2^{flox/+} and E-Syt2^{flox/flox} mice (3) PCR was used to identify E-Syt2^{flox/flox} mice. Then, these mice have been crossed with CD4-Cre transgenic mice that express Cre recombinase in CD4+/CD8+ double-positive (DP) thymocytes. As expected, this crossbreeding generated heterozygous E-Syt2^{flox/+}, CD4Cre and homozygous E-Syt2^{flox/flox}, CD4Cre mice. (See Hirotsune, S. et al. Graded reduction of Pafah1b1 (Lis1) activity results in neuronal migration defects and early embryonic lethality. Nat Genet 19, 333-339 (1998). 2-Chen, L. et al. Gly369Cys mutation in mouse FGFR3 causes achondroplasia by affecting both chondrogenesis and osteogenesis. J Clin Invest 104, 1517-1525 (1999); and Meyers, E. N., Lewandoski, M. & Martin, G. R. An Fgf8 mutant allelic series generated by Cre- and Flp-mediated recombination. Nat Genet 18, 136-141 (1998).

All mice were housed under specific pathogen-free conditions at the Laboratory Animal Facilities of the Institut Pasteur. Mice were used for experiments at 6-16 weeks of age, and experiments were conducted following the guidelines provided by the Animal Care and Use Committee of the Institut Pasteur, and in accordance with French law.

Cell Isolation and Flow Cytometry Analysis

Total splenocytes, lymph node and thymus were prepared by gentle dissociation using mesh filters followed by erythrocyte lysis using NH₄Cl. To analyze cell phenotype, we used fluorescence conjugated CD44 mAb (BD Pharmingen), anti-CD25 (eBioscience), anti-CD62L (eBioscience), anti-CD4 (eBioscience), anti-CD19 (eBioscience), anti-69 (BD Pharmingen). All washings and reagent dilutions were made with PBS containing 2% fetal calf serum (FCS). A FACSFortessa Flow cytometer interfaced to the FACS-Diva software (BD Bioscience) was used to acquire data that was analyzed using FlowJo software.

Histology and Immunohistochemistry

For Morphological Analysis:

The skin and the spleen were removed and immediately fixed in 10% neutral buffered formalin. Tissue samples from these organs were embedded in paraffin; 5-μm sections were cut and stained with hematoxylin and eosin (HE).

For Immunohistochemistry:

Tissue samples from the spleen were fixed in JB fixative (zinc acetate 0.5%, zinc chloride 0.05%, and calcium acetate 0.05% in Tris buffer at pH 7) for 48 hours and then embedded in low-melting point paraffin (Poly Ethylene Glycol Distearate; Sigma, USA). 5-pm thick sections were deparaffinized in absolute ethanol, air dried, and used for immunolabeling. The following primary antibodies were used: anti-CD3 (Rabbit antibody anti-human, 1/75e, A0452, DAKO, Carpinteria, Calif.), and anti-CD45R (Rat anti-mouse, clone B220, 1/40e, RM2600, Invitrogen, Carlsbad, Calif., USA).

IL-2 Production

CD4+ cells purified from the spleen were diluted in RPMI with 10% FCS at 10⁶ cells/well. Cells were left unstimulated or stimulated with anti-CD3/CD28, 5 mg/ml and PMA at 5 ng/ml and Ionomycin at 1 pM for 24 hr, supernantants were tested for IL-2 production by ELISA.

In Vitro TCR Stimulation and Intracellular Staining and Subcellular Fraction Experiments

Jurkat cells were incubated 30 min at 4° C. with 10 pg/ml UCHT1 anti-CD3 mAb and 10 pg/ml CD28.2 anti-C28 antibody and then for the indicated period of time at 37° C. with RPMi containing 10 pg/ml goat anti-mouse IgG. Cells (10⁷) were incubated for the indicated time at 37° C. in 1 ml of RPMI containing 5 ng/ml PMA (Sigma) and 1 pM calcium ionophore (Sigma).

To purify CD4+ cells from total cells, multi Ab kit was used (Dynabeads®). Purified CD4+ cells (5×10⁴ cells/well) were labeled with CFSE (Invitrogen) according to the manufacturer's instructions (especially incubated 30 mM with 1.5 pg/ml biotinylated anti-CD3 (145-2C11) and 1 pg/ml anti-CD28 antibodies at 4° C. and then at 37° C. for the indicated period of time with RPMI and streptavidin). Total splenocyte diluted in RPMI1640 with 10% FCS were added (10⁶ cells/well). In some cases, cells were left unstimulated or stimulated with cytokines (IL-2 at 10 ng/ml plus CD3/CD28 at 10 μg/ml; PMA at 30 μg/ml; Ionomycin at 500 μg/ml) for 18 hr. After surface staining and incubation with LIVE/DEAD Fixable reagent (Invitrogen), cells were fixed in 4% paraformaldehyde (PFA) and processed for intracellular cytokine detection by using permeabilization buffer. Cells were stained with PerCP-conjugated anti-Foxp3 (eBioscience), PE-Cy7 labeled anti-T-bet mAb (eBioscience) and APC-conjugated GATA3 (eBioscience) in permeabilization buffer before extensive washing in PBS and analysis. Subcellular fractionation experiments were performed as described previously. Briefly, cells in 20 mM HEPES pH 7.9, 1.5 mM MgCb, 60 mM KCl, and protease inhibitors (Roche) were mechanically permeabilized with a 27G^{1/2} syringe (Becton Dickinson). Nuclei and unbroken cells were removed by a 1,000 g centrifugation. Crude heavy membrane fractions (HM) were obtained after a 10,000 g centrifugation. The remaining supernatants were further cleared with a 25,000 g centrifugation for an additional 30 min to obtain the cytosolic fractions.

Expression and Purification of Recombinant Proteins

pGEX-4T, pGEX-4T-SH3 (CARMA1) and pGEX-4T-SH3 (C-ter Vav; a gift from Dr. Nadine Varin-Blank (INSERM U978, France) were transformed into E. coli cells (BL21). Expression was induced by addition of 1 mM IPTG for 4 hours. The GST-fusion proteins were purified on glutathione-sepharose 4B beads (GE Healthcare). In vitro translated HA-NEMO and HA-E-Syt2 were produced from TNT coupled Reticulocyte Lysate Systems according to the manufacturer instructions (Promega).

Two-Hybrid Screen in Yeast

A cDNA corresponding to the SH3 domain of CARMA1 (amino acid 765-832) was cloned into the C terminal LexA DNA-binding domain (DBD) vector pB27. The resulting plasmid pB27-SH3carma1 was used as bait in a two-hybrid screen of a human CD4+/CD8+ thymocytes cDNA library (Hybrigenics) in Saccharomyces cerevisiae HF7c. Valuable clones were isolated following two-hybrid interaction analyses (Hybrigenics).

### Example 1: E-Syt2 Interacts With CARMA1 Upon T Cell Activation

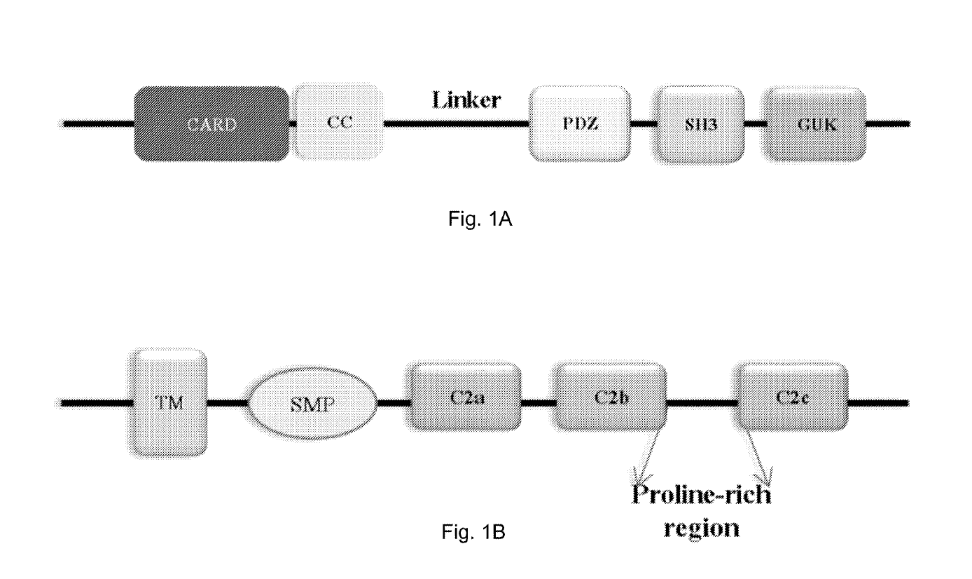

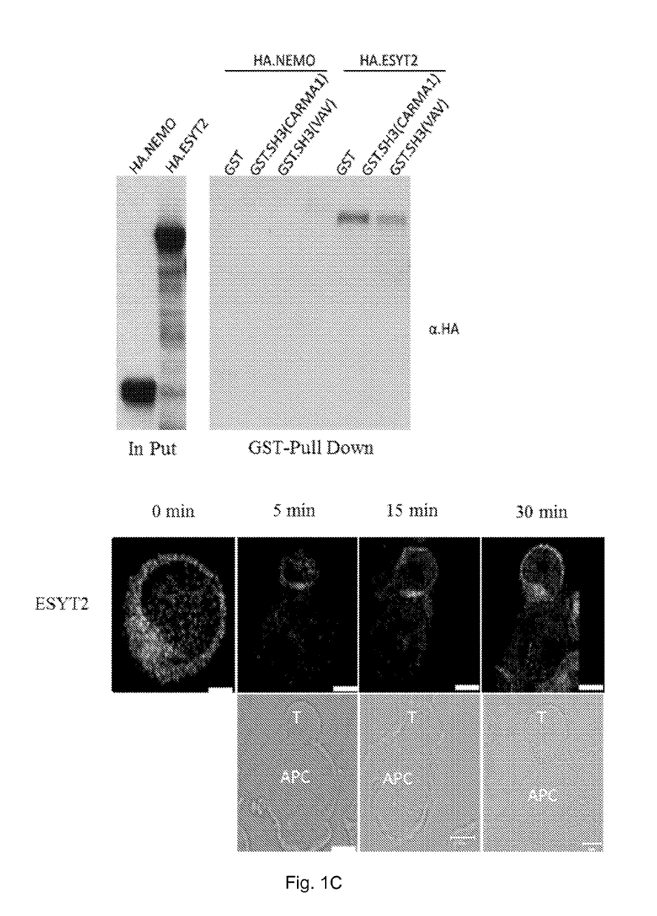

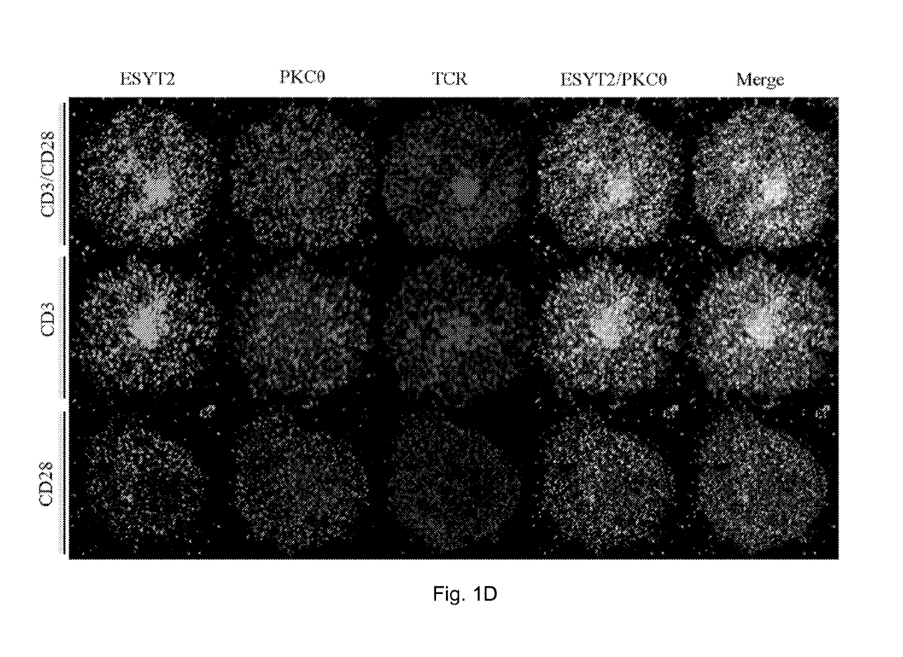

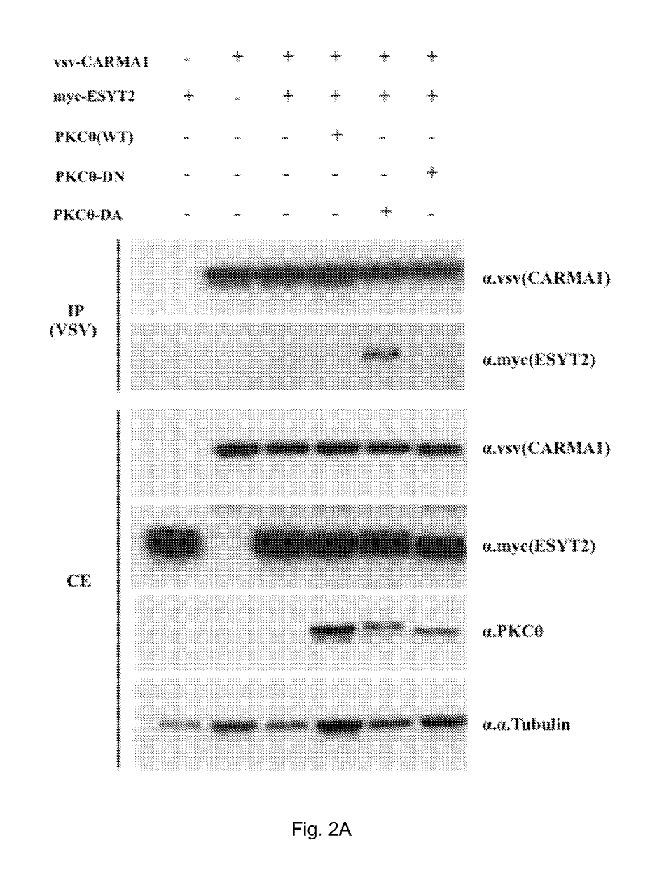

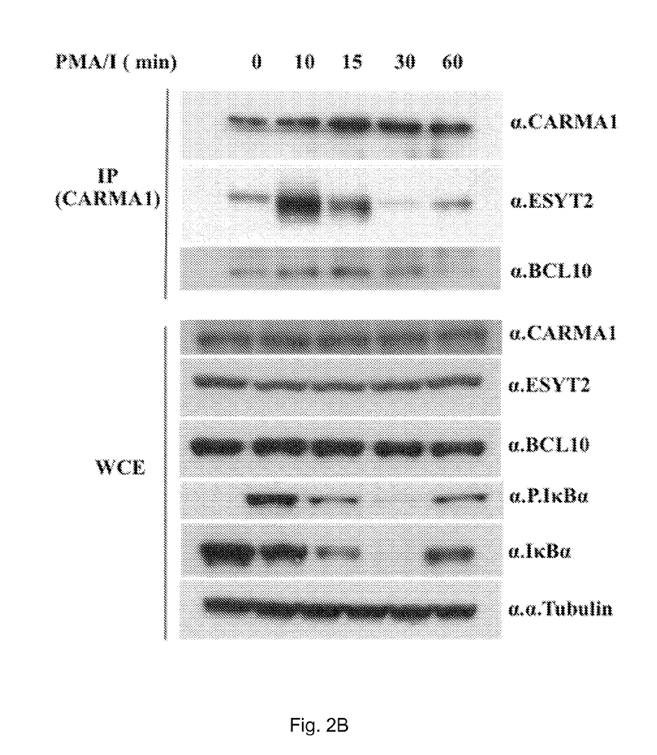

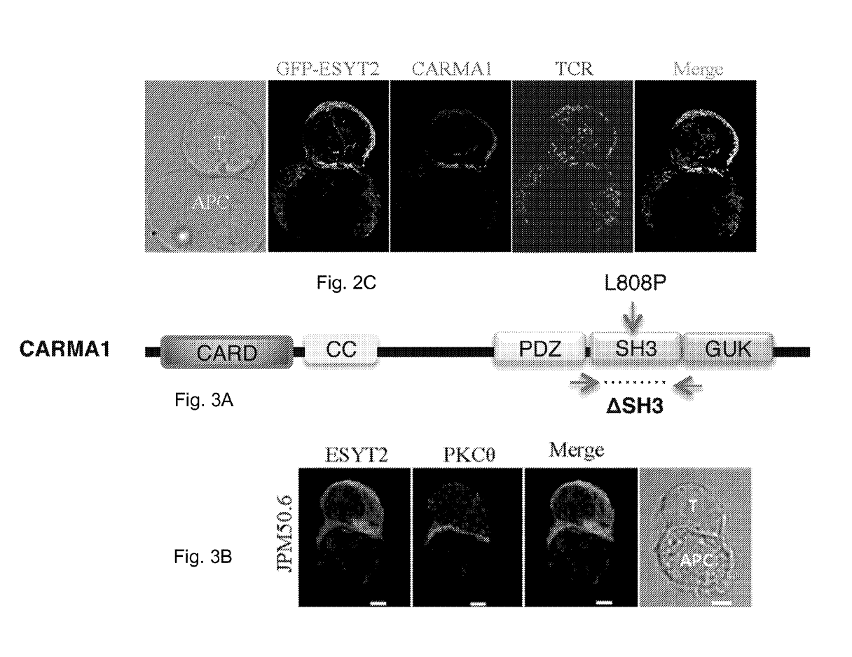

The molecular mechanisms responsible for the recruitment and activation of the CBM complex in the IS are not fully understood. Importance of the localization of CARMA1 in the IS has been emphasized by the finding that a mutation of CARMA1 in its SH3 domain, which consist on a substitution of Leucine 808 by a Proline (L808P) exhibits defective recruitment to the IS and impaired NF-κB activation. This suggests that CARMA1 is targeted to the immunological synapse through the interaction of its SH3 domain with a membrane component.²⁷ Using a yeast two-hybrid approach with a human CD4+/CD8+ thymocyte cDNA library and the CARMA1 SH3 domain as bait, we identified a total of 31 putative CARMA1 interacting proteins. Among those only nine proteins displayed high to medium interaction confidence (Table 1). We focused our attention on the Extended-Synaptotagmin 2 (ESYT2) protein because it presents a similar tissue-specific expression pattern to CARMA1 including high expression in T-cells (geneCards human gene database; http://www.genecards.org/cgi-bin/carddisp.pl?gene=ESYT2&search=e-syt2) and contains a proline-rich domain that could interact with the SH3 domain of CARMA1, as well as a C2 domain likely responsible for its cell membrane association. In addition, ESYT2 was identified in a functional genomic screen searching for new positive or negative regulators of T cell activation following TCR stimulation³⁹. The specificity of the interaction between CARMA1 SH3 domain and ESYT2 was examined in vitro. To this end, we performed GST-pull-down assays (FIG. 1.c) using in vitro translated HA-ESYT2 and HA-NEMO (as control) in reticulocytes cell lysates and GST-fusion proteins encompassing GST alone or fused to CARMA1 or Vav SH3 domains. These experiments did not reveal any interaction between HA-NEMO (our negative control) and any of the GST fusion proteins (FIG. 1c) or between HA-ESYT2 and GST alone; however, we observed a preferential interaction between ESYT2 and the SH3 domain of CARMA1 rather to that of Vav. Since we observed that ESYT2 interacts with CARMA1 SH3 domain we sought to investigate whether ESYT2 could also interact with the full-length CARMA1 protein. To examine this possibility, HEK 293T cells transfected with plasmids encoding CARMA1 and ESYT2 together with different constructs of PKCθ, referred as PKCθ wild type (WT), PKCθ dominant negative (DN) or PKCθ constitutively active (DA) were subjected to co-immunoprecipitation experiments followed by Western blotting analysis (FIG. 2a). We did not observe any interaction between ESYT2 and CARMA1 alone. However, we could detect an interaction between the two proteins when the cells were co-transfected with the dominant active form of PKCθ DA. Since it has been shown that CARMA1 undergoes a change of conformation, upon phosphorylation by PKCθ, which mediates its association with its interacting proteins^{40,41}, our observation suggests that PKCθ-mediated CARMA1 phosphorylation is required for CARMA1 to interact with ESYT2. To examine this hypothesis in more physiological conditions, we performed co-immunoprecipitation experiments of endogenous proteins in Jurkat cells stimulated with Phorbol 12-Myristate 13-acetate (PMA) and Ionomycin or left untreated. As shown in FIG. 2b, the signal corresponding to endogenous ESYT2 was strongly enhanced in CARMA1 immunoprecipitates after 10 minutes of PMA/ionomycin treatment, indicating that CARMA1 must be in an “active conformation” to interact with ESYT2. In addition, we found that CARMA1 associates with E-SYT2 with the similar kinetic as with BCL10.

TABLE 1
interacting Partners of CARMA1 SH3 domain in the two hybrid screen
Yeast two-hybrid screening using CARMA1 SH3 domain as bait and
human CD4+/CD8+ thymocytes cDNA as library. Our analyses revealed
32 candidates with 9 proteins displaying high to medium interaction
among which Extended-Synaptotagmin 2 protein (E-Syt2). The number
of identified clones and the size of insert are given for each protein.

| | | Size of insert | |
|---|---|---|---|
| | Number of | (Start . . . | |
| Gene | clones | stop aa) | Protein |
| Human BTBD15 | 6 | 699-1633 | ZBTB44 (DNA binding |
| | | | protein) |
| Human STAT1 | 5 | 276-1214 | STAT1 (signal transduc- |
| | | | er and activator of |
| | | | transcription 1) |
| Human sub2.3 | 32 | 63-1147 | PCBP1 (RNA binding |
| | | | protein) |
| Human genematch | 2 | 54-587 | |
| GID: 12666198 | | | |
| Human KIAA1228 | 2 | 1587-2026 | ESYT2 |
| Human UPB1 | 5 | 210-892 | UPB1 (beta- |
| | | | ureidopropionase) |
| Human TFCP2 | 6 | 243-949 | TFCP2 (DNA binding |
| | | | protein) |
| Human FLNB | 3 | 4599-5322 | FLNB (Filamin B: Actin |
| | | | binding protein) |
| Human LTA4H | 3 | 39-567 | LTA4H (leukotriene A-4 |
| | | | hydrolase) |

### Example 2: ESYT2 Translocates to the IS and to Microclusters and Colocalizes with CARMA1 in an SH3-Dependent Manner

Since our results indicate that ESYT2 interacts with CARMA1 in response to TCR stimulation, we hypothesized that ESYT2 could recruit CARMA1 to the IS through its SH3 domain. Before testing this hypothesis, we first investigated whether ESYT2 could be recruited to TCR signaling complexes at the IS. To this purpose, GFP-ESYT2 expressing Jurkat cells were stimulated by staphylococcal enterotoxin E (SEE)-coated Raji cells under conditions that lead to TCR and downstream signaling components clustering at the T cell-APC contact zone that can be visualized by immunofluorescence and confocal microscopy (FIG. 1c)⁴². Under these conditions, we observed a marked accumulation of ESYT2 at the immunological synapse, where it localizes together with the TCR and CARMA1, that peaked after five minutes of conjugate formation and decreased after 30 min of conjugates formation. Interestingly, TCR and downstream signaling molecules were previously shown to aggregate onto dynamic microclusters (MCs) that move toward the center of the contact site to form the cSMAC. To examine whether ESYT2 did also organize in microclusters, Jurkat T cells were allowed to settle onto glass coverslips coated either with anti-CD3, anti-CD28 or both (FIG. 1d).

Notably, we observed that ESYT2 formed MCs following co-stimulation with anti-CD3 and anti-CD28 or anti-CD3 alone (FIG. 1d). However, ESYT2-containing clusters were less apparent upon stimulation with anti-CD28. PKCθ has recently been observed to form MCs that colocalize with TCR upon CD28 stimulation⁴³. Accordingly, our results indicate that CD28 plays a critical role in the segregation of PKCθ to TCR MCs. Interestingly, we observed that CD28 was less potent to recruit ESYT2 than PKCθ into MCs. Interestingly, ESYT2 and PKCθ did not co-localize but rather accumulates and localizes in closed MCs.

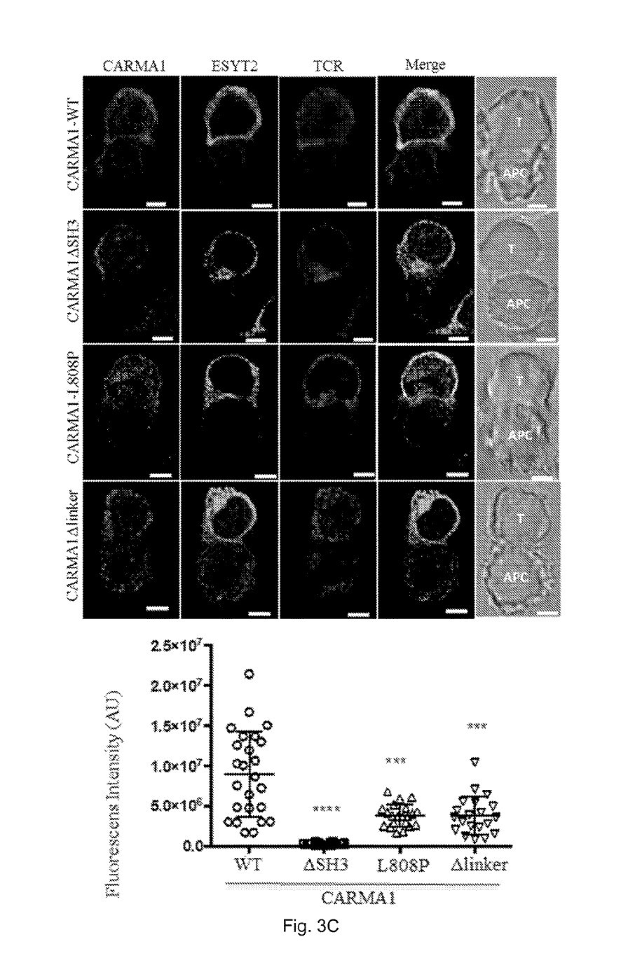

CARMA1 is recruited to the immunological synapse through its SH3 domain as this change of localization is prevented by a point mutation of CARMA1 in its SH3 domain²⁷. To confirm this result, we stably transfected JPM50.6, a Jurkat-derived T cell clone deficient for CARMA1 expression⁴⁴, with plasmids encoding either wild-type (CARMA1-WT), an SH3 deletion mutant (CARMA1-ASH3), a point mutation of the SH3 domain (CARMA1 L808P), or a deletion of the linker domain (CARMA1-Alinker) of CARMA1 and valuable clones were screen under limiting conditions. We then investigated the cellular distribution of the CARMA1 derivatives expressed in JPM50.6 after 10 min of co-culture of the JPM50.6 stable clones with SEE-coated Raji cells. As expected, anti-CARMA1 did not detect any signal in JPM50.6 cells and in reconstituted cells, CARMA1-WT accumulates at the T cell/APC interface together with ESYT2 and the TCR as shown in FIG. 3c. Moreover, deletion or point mutation of CARMA1 SH3 domain impaired the localization of CARMA1 at the IS and its colocalization with ESYT2. Surprisingly, CARMA1-Alinker was also not recruited to the immunological synapse. These results were analyzed more deeply through the quantification of CARMA1 at the immunological synapse from at least 20 cells in each condition (FIG. 3). Altogether, these results show that not only the SH3 domain, but surprisingly also the linker domain of CARMA1 are important for the recruitment of the protein to the immunological synapse. These results also confirm that a point mutation of CARMA1 in its SH3 domain is sufficient to disrupt the recruitment of CARMA1 to the synaptic plasma membrane.

### Example 3: ESYT2 Controls CARMA1 and BCL10 Translocations to the IS and to Microclusters

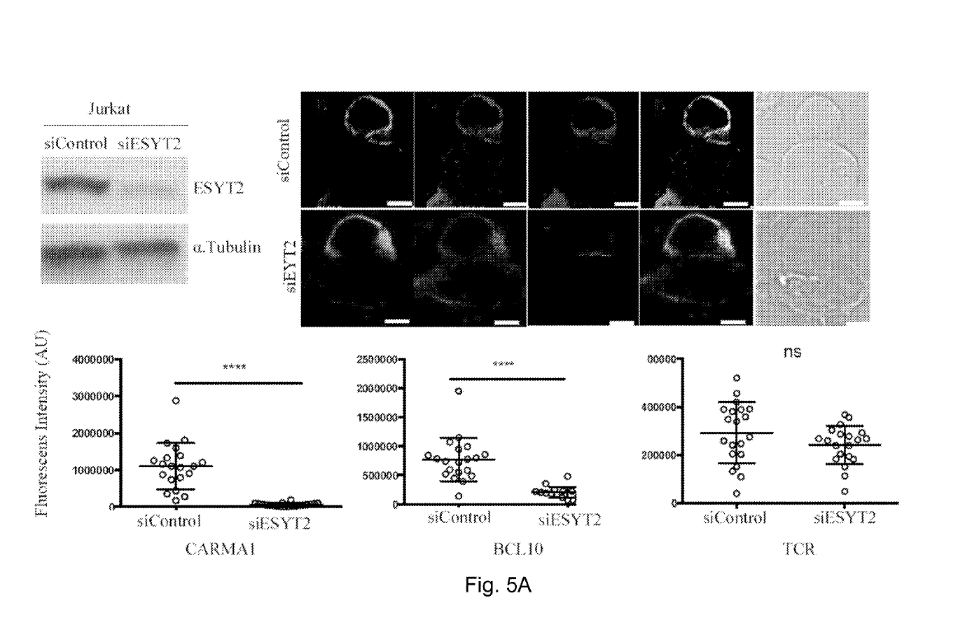

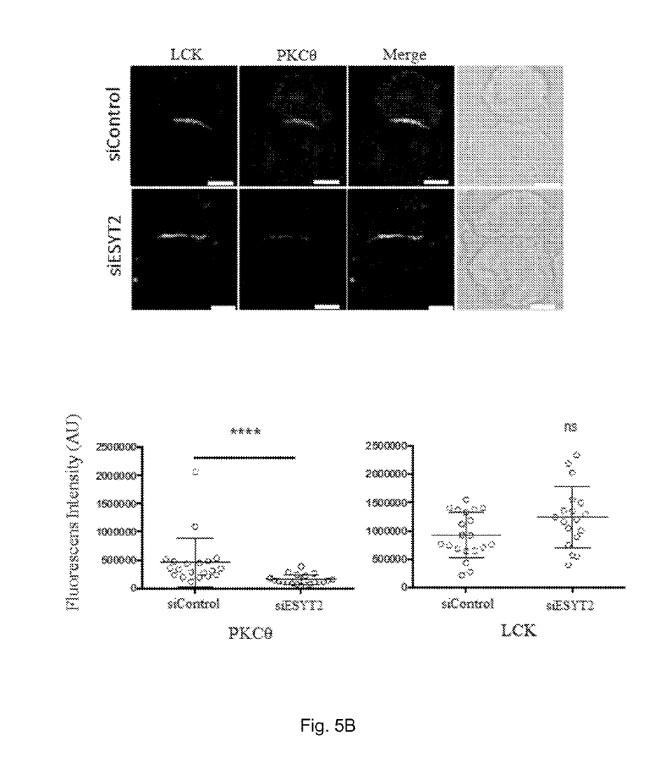

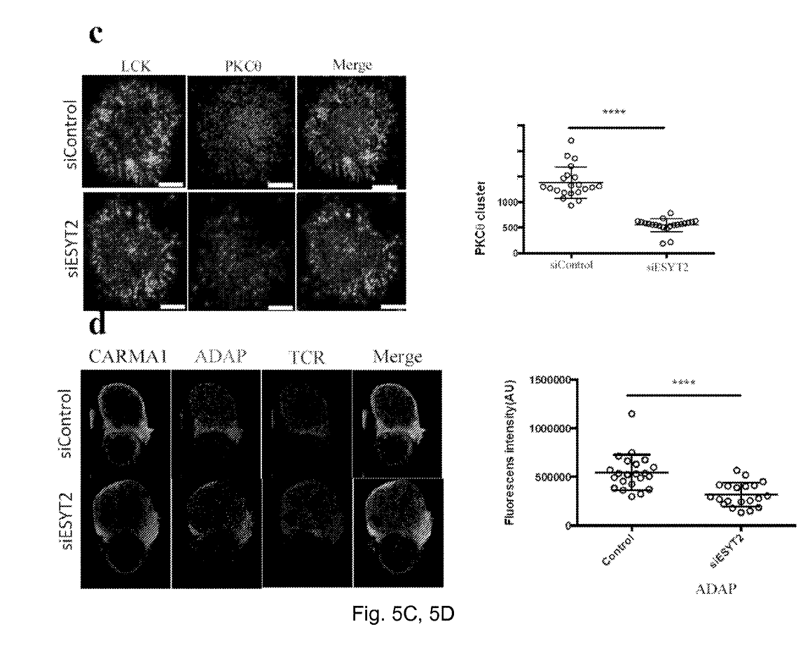

It has been previously demonstrated that the SH3 domain of CARMA1 is responsible for its recruitment to the immunological synapse and we showed that this domain also interacts with ESYT2. This protein constitutes therefore a new component of the TCR signalosome and we sought to determine whether it could target CARMA1 and the CBM complex to the immunological synapse. In order to assess this hypothesis, we investigated the localization of CARMA1 and BCL10 at the immunological synapse in absence of ESYT2 using a siRNA approach. Conjugates between ESYT2-depleted Jurkat clones and Raji cells loaded with SEE were formed for 10 minutes, fixed and labeled with Abs specific for CARMA1, BCL10 and TCR (FIG. 5a). As expected, stimulation of Jurkat cells transfected with a control siRNA or untransfected cells led to the redistribution of these molecules from the cytosol to the IS. In contrast, the translocation of CARMA1 and BCL10 to the IS was strongly diminished in Jurkat cells depleted for ESYT2. However, ESYT2 depletion did not affect the redistribution of the TCR from the cytosol to the IS since we could observe an accumulation of the TCR at the T/APC interface in Jurkat cells transfected either with the control siRNA or ESYT2 siRNA. These experiments also indicates that the lack of recruitment of CARMA1 and BCL10 observed in ESYT2-depleted cells cannot be attributed to a defect of IS formation since upstream signaling proteins can be recruited to this location. We further investigated this defect by scoring the percentage of T cells that exhibited a translocation of these proteins. This quantification demonstrate that in Jurkat cells transfected with the control siRNA, CARMA1 and BCL10 translocation were normally observed. Similar quantification indicated that ESYT2 depletion prevented the translocation of these proteins in the IS. These quantification also confirmed that ESYT2 depletion specifically prevents IS recruitment of these proteins the conjugates displayed a normal redistribution of the TCR and Lck from the cytosol to the IS independently of ESYT2 expression.

### Example 4: ESYT2-Dependent and CARMA1-Independent Translocation of PKCθ to the Immunological Synapse and to MCs

In addition to its involvement in BCL10 recruitment to the IS, CARMA1 may also be important for the IS localization of PKCθ following TCR stimulation^{45,46} Our results clearly indicate that PKCθ is recruited to the IS in JPM50.6 indicating that PKCθ localizes to the IS independently of CARMA1 expression (FIG. 5a). Although Wang and colleagues did not reach to this conclusion²⁷, our finding is in agreement with previous reports^{20,28}. However, the recruitment of PKCθ to the immunological synapse requires the expression of ESYT2. Indeed, we observed a strong decrease of PKCθ detection at the center of the immunological synapse in ESYT2-depleted Jurkat cells (FIG. 5b). In contrast, the recruitment of Lck, which forms a tripartite complex together with CD28 and PKCθ was not affected in Jurkat transfected either with ESYT2 siRNA or control siRNA.

The critical role of E-Syt2 in retaining PKCθ in supramolecular clusters was demonstrated using glass coverslips coated with anti CD3 and anti CD28 (FIG. 5c). while almost all Jurkat cells transfected with a control siRNA showed the presence of PKCθ clustered both at the periphery and at the center of the immunological synapse, PKCθ presented a deficient clustering and absence of internalization in ESYT2-depleted cells.

### Example 5: ESYT2 Controls the Recruitment of ADAP to the Immunological Synapse

Since CARMA1 has been shown to associate with the adaptor protein ADAP (adhesion and degranulation promoting adapter protein^{14,15}, we next examined whether the recruitment of ADAP to the IS might be regulated by ESYT2. We compared the intracellular distribution of ADAP to that of CARMA1 in control and ESYT2-depleted Jurkat cells co-cultured with SEE-loaded Raji cells (FIG. 5d). CARMA1 and ADAP immunostaining showed that the two proteins colocalize in membrane protrusions and in the IS and were mostly excluded from its center in E-Syt2-depleted Jurkat cells. Quantification analysis of ADAP localized at the immunological synapse in control and ESYT2-depleted cells indicated that the absence of ESYT2 impacted on the recruitment of ADAP to the synapse. As expected, our results also demonstrate that the TCR clustering at the synapse is unaffected in control and ESYT2-depleted Jurkat cells.

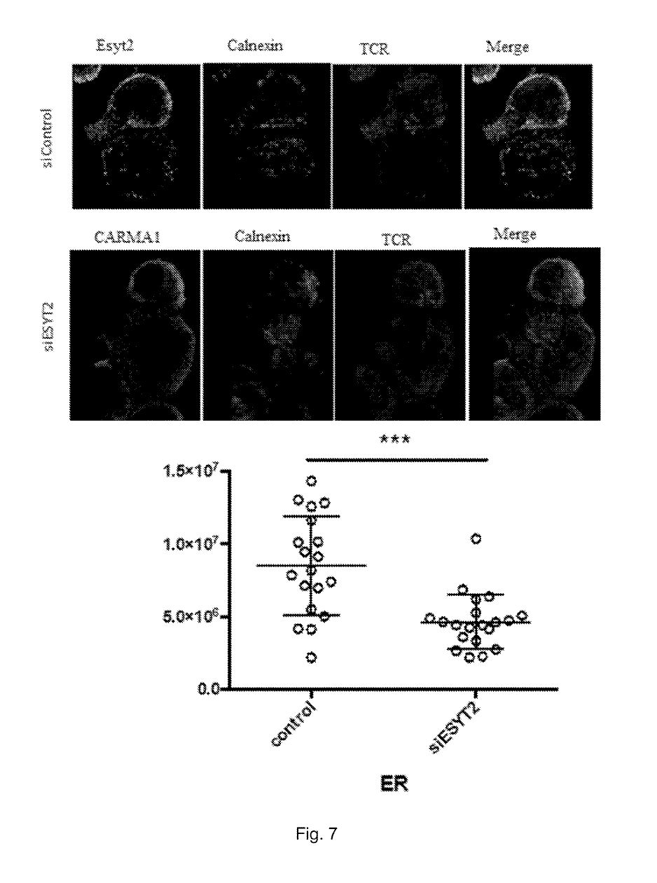

It has been recently shown that Extended-Synaptotagmins family of proteins (ESYTs) are endoplasmic reticulum (ER) anchored proteins that mediate contacts with the plasma membrane³⁵. Also, many studies showed that following synapse formation, the ER is adjacent to this location. We thus sought to determine whether loss of ESYT2 could alter the distribution of the ER at the immune synapse. To this end, SEE-coated Raji cells were mixed with control or ESYT2-depleted Jurkat cells and were fixed and stained with anti-Calnexin, an integral protein of the ER (FIG. 7). In control stimulated Jurkat cells, the ER was diffusely distributed throughout the cytoplasm but predominantly localized at the IS while we found that the ESYT2 depletion caused a decrease of endoplasmic reticulum detection at the IS. Therefore, ESYT2 induces the retargeting of the endoplasmic reticulum to IS.

Altogether, our experiments demonstrate that ESYT2 orchestrates the selective recruitment of PKCθ, CARMA1 and BCL10, ADAP and the ER at the IS. Since PKCθ redistribution from the cytosol to the central IS is affected by the absence of ESYT2, but not by the absence or lack of recruitment of CARMA1, it is most likely that E-Syt2 does not recruit PKCθ to the IS synapse via its SH3 domain that is required for its association with CARMA1, but through another, distinct domain.

### Example 6: Expression, Localization and Heterodimerization of E-Syt1 and E-Syt2 in Jurkat Cells

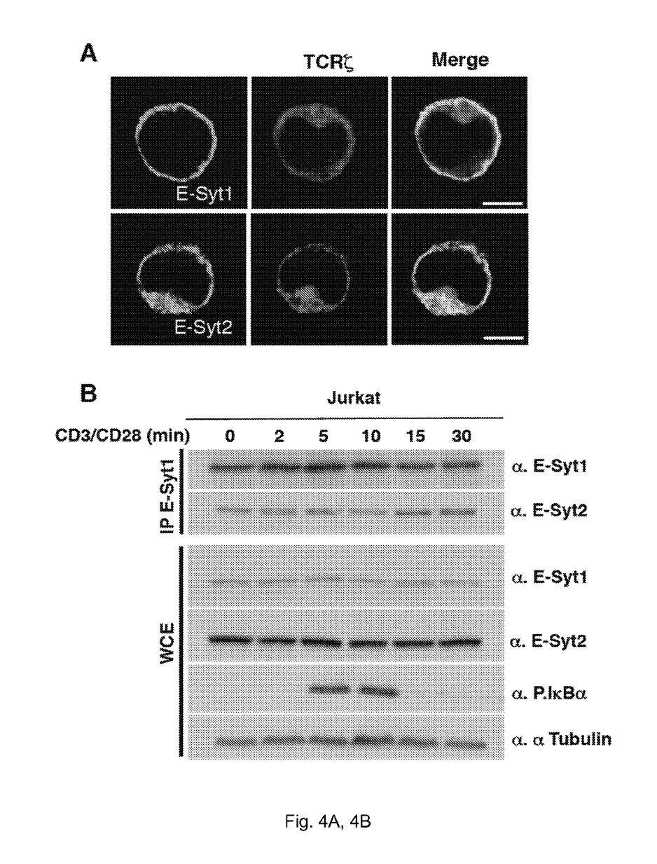

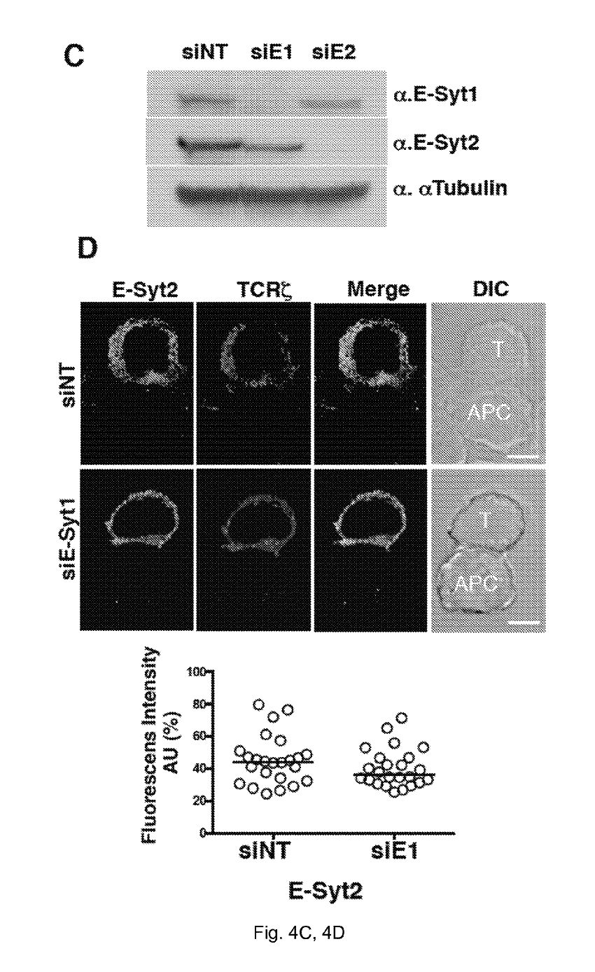

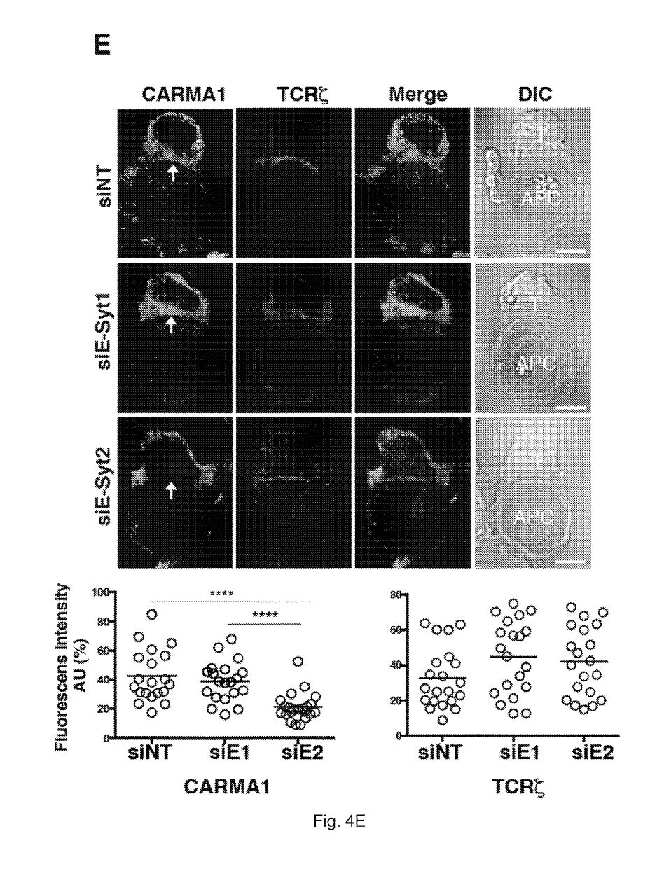

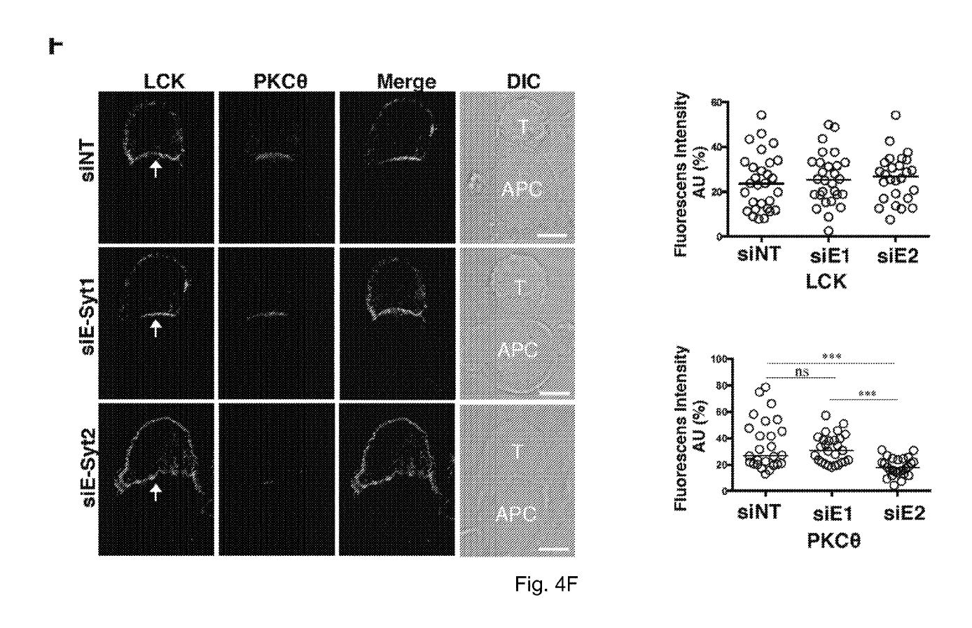

Since only E-Syt2 and E-Syt1 are expressed in T cells, we focused on the dynamic of heteromeric complex formation upon TCR stimulation and their respective importance for the recruitment of signaling components to the IS. In Jurkat cells, endogenous E-Syt1 was predominantly expressed at the plasma membrane, while E-Syt2 had a broad cellular localization (FIG. 4A), in contrast to their exogenous distribution. As both proteins constitutively interact (FIG. 4B), we questioned whether E-Syt1 could be required for the localization of E-Syt2 and the CBM complex to the IS. However, silencing E-Syt1 expression did not affect E-Syt2 localization to the IS and, unlike E-Syt2, did not impair PKC6 or CARMA1 localization (FIG. 4C, D, E, F). Altogether, our experiments demonstrate that E-Syt2 selectively and specifically orchestrates the recruitment of PKC6 and the CBM signalosome to the IS.

### Example 7: Generation of E-Syt2−/− Mice and Mice with an E-Syt2 Cre/LoxP Conditional Knockout Allele

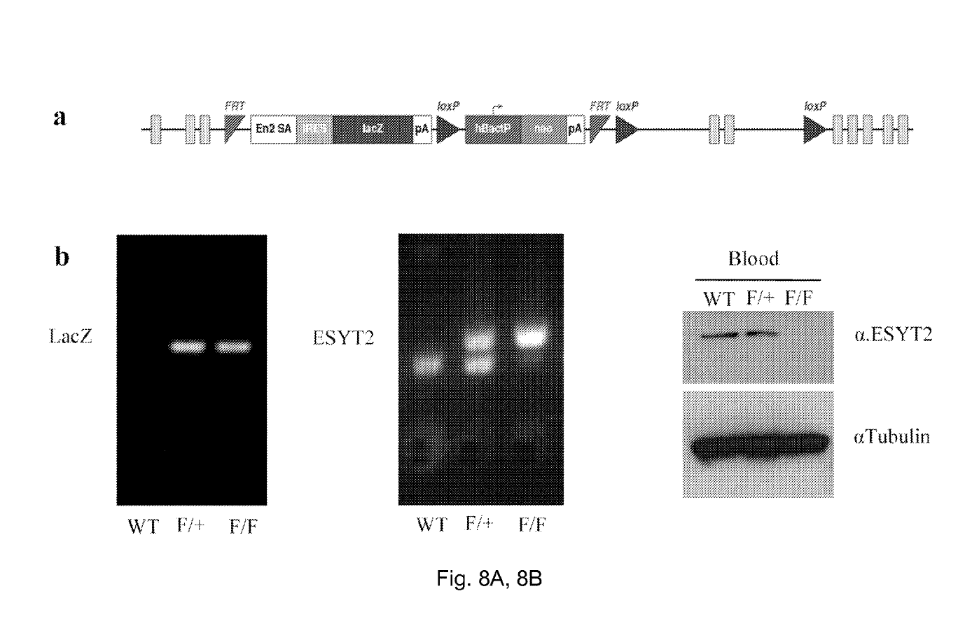

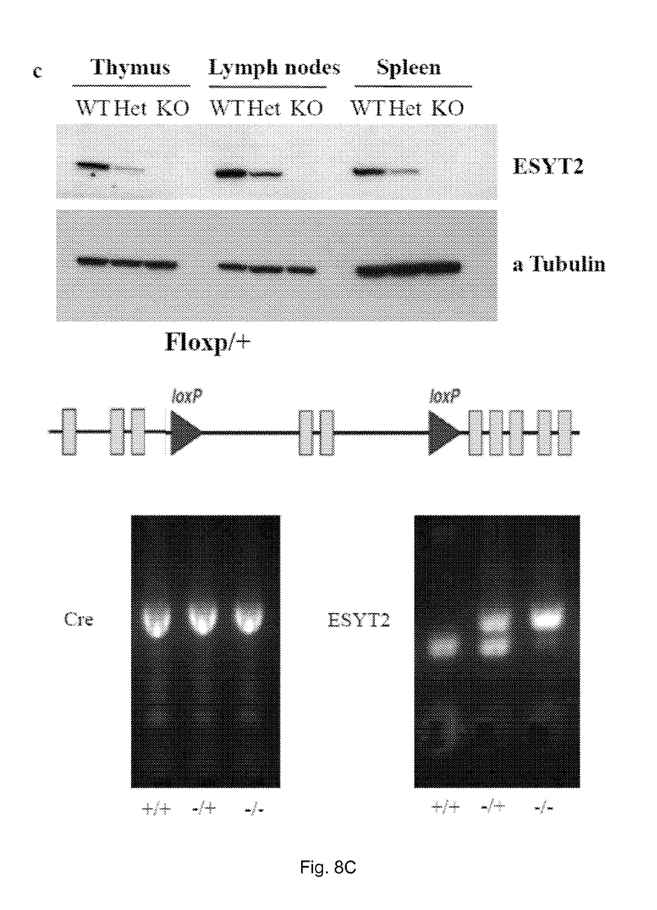

Mice deficient for PKCθ, BCL10, or CARMA1 show impaired development and activation of thymocytes, as well as, impaired Treg cell development⁹. To address whether ESYT2 is required for these developmental steps, we generated and analyzed several modified E-Syt2 alleles (see Methods) in mice on the C57BL/6 background, in particular we generated mice carrying a conditional E-Syt2 knockout allele in the thymus using the Cre-lox system. A gene-targeting vector, in which two LoxP sites were inserted into the introns flanking exons of E-Syt2, was constructed (EUCOMM-KOMP CSD) (FIG. 8a). The targeting vector also includes a PGKneo cassette flanked by a third loxP site. In this way, existence of Cre recombinase would lead to deletion of exons 4 and 5, and subsequent inactivation of E-Syt2. Following electroporation of linearized targeting vector into embryonic stem (ES) cells from the C57BL/6N strain, neomycin-resistant colonies were screened for homologous recombinants using polymerase chain reaction (PCR). E-Syt2^{FIN/+} ES cells were injected into blastocysts from the BALB/cN strain and mice with germline transmission of the E-Syt2 allele were obtained. E-Syt2^{flox-neo/+} heterozygous mice were intercrossed to generate E-Syt2^{flox-neo/flox-neo} mice. PCR with primers shown in FIG. 8a were used to identify E-Syt2^{flox-neo/flox-neo}. RT-PCR analysis revealed that there was no ESYT2 expression in E-Syt2^{flox-neo/flox-neo}. The homozygous-null mutant mice had no detectable ESYT2 protein expression in Thymus, lymph nodes and spleen, while the expression of ESYT2 was half-reduced in heterozygous mice (FIG. 8c). This indicates that the presence of neomycin gene (neo) blocked the normal splicing of ESYT2 mRNA as previously described for other genes^{47,48}, and that homozygous E-Syt2^{flox-neo/flox-neo} mice showed phenotypes similar to those of ESYT2-null (E-Syt2−/−) mice.

E-Syt2^{FLN/FLN} mice were viable, fertile and born at the expected Mendelian ratio. The Frt site-flanked drug selection cassette (lacZ-neo) was remove by crossing E-Syt2^{flox-neo/flox-neo} mice with transgenic Flp mice expressing Flp recombinase to obtain E-Syt2^{flox/+} and E-Syt2^{flox/flox} mice⁴⁹. PCR was used to identify E-Syt2^{flox/flox} mice (FIG. 8b). Then, these mice have been crossed with CD4-Cre transgenic mice that express Cre recombinase in CD4+/CD8+ double-positive (DP) thymocytes. The offspring were identified by PCR. As expected, this crossing breeding generated heterozygous E-Syt2^{flox/+}, CD4Cre and homozygous E-Syt2^{flox/flox}, CD4Cre mice.

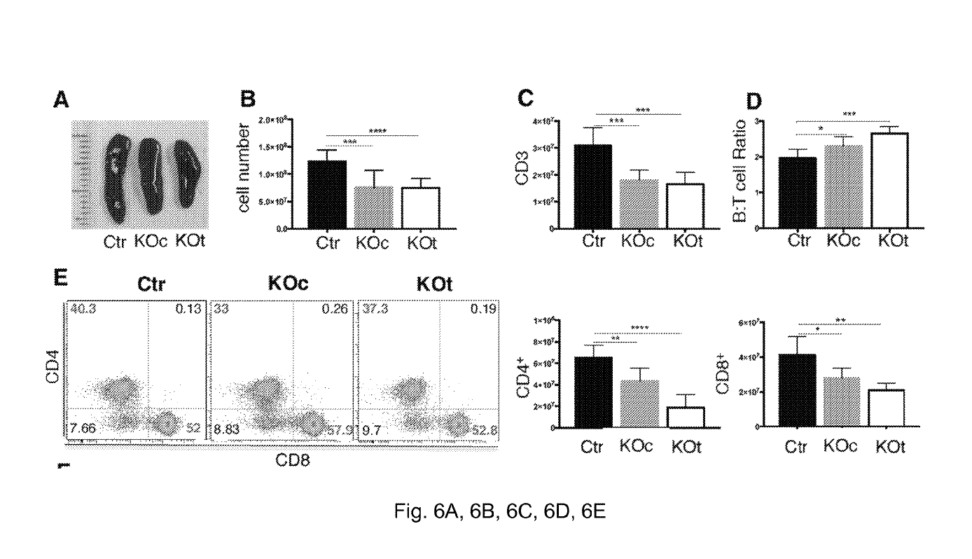

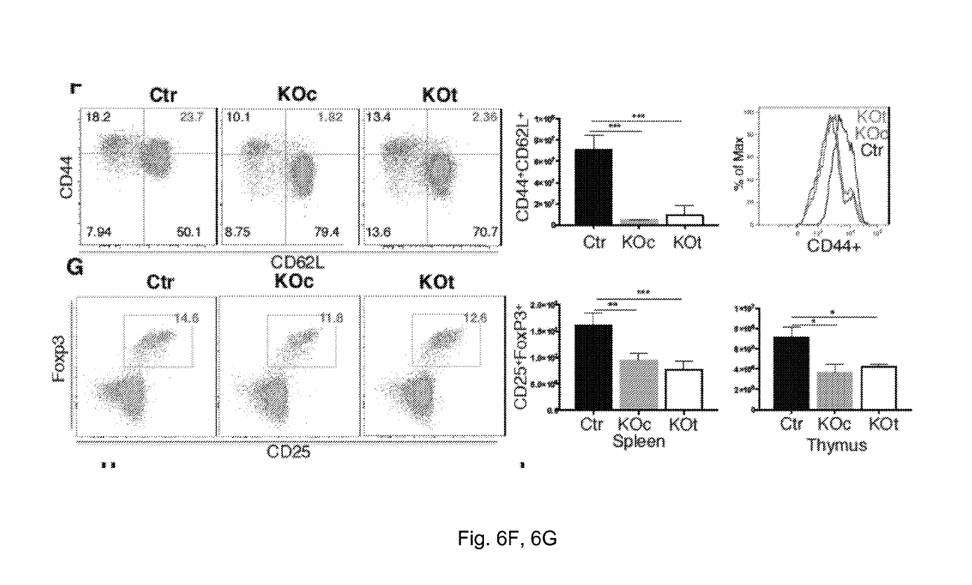

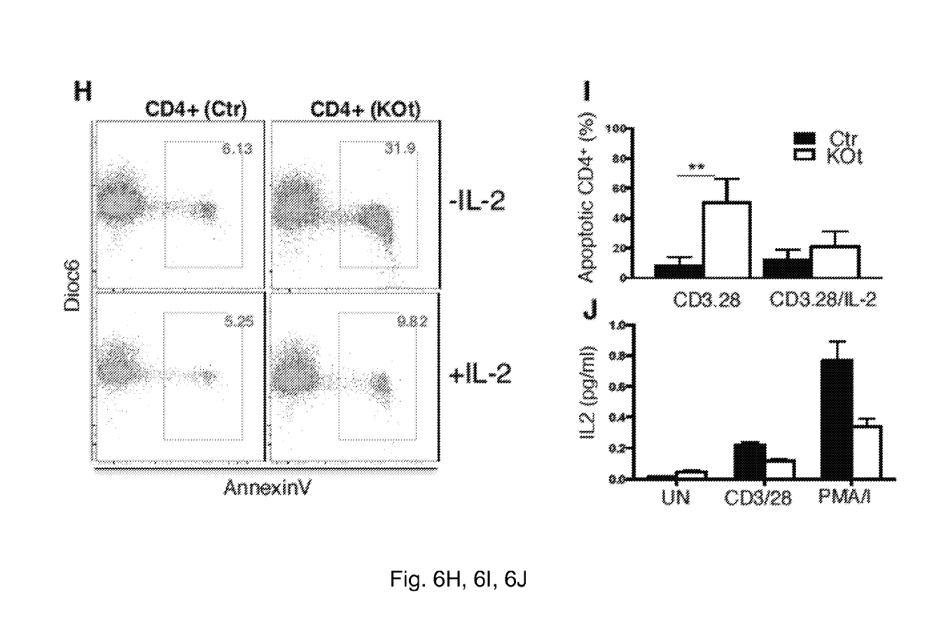

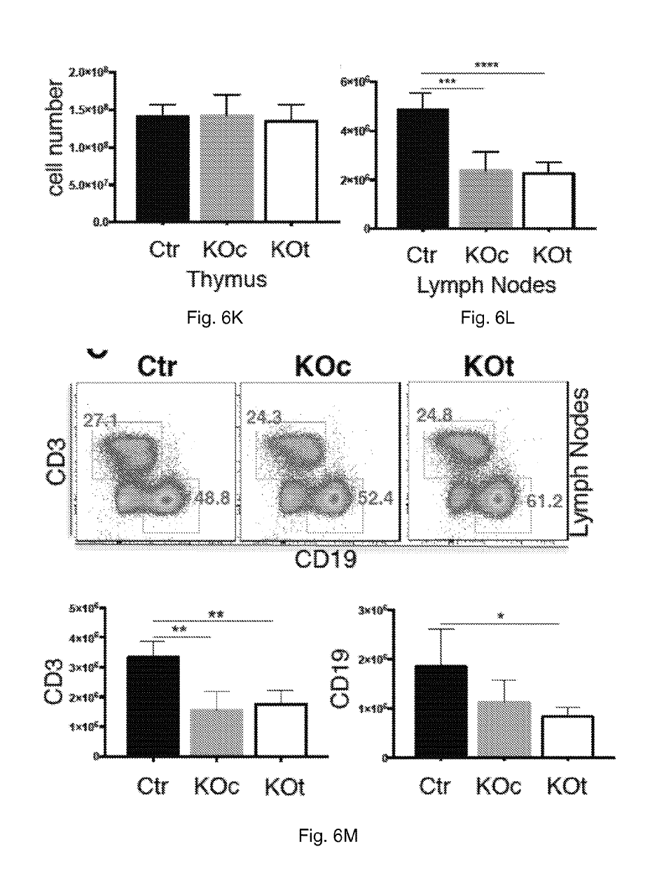

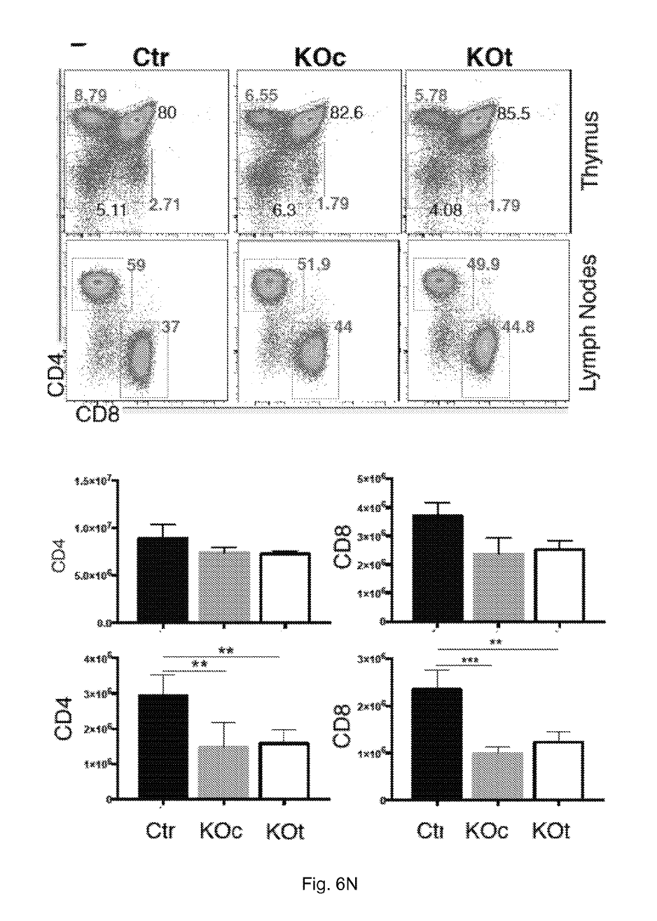

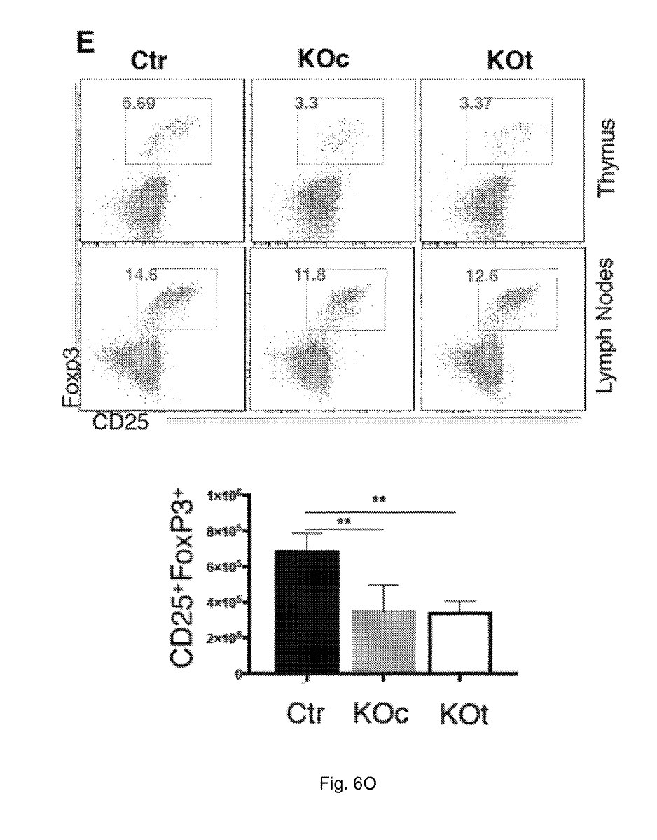

Lymphocytes purified from thymus, lymph nodes and spleen of E-Syt2^{flox/flox} CD4-Cre mice did not express detectable amount of E-Syt2 compared to CD4-Cre wild-type controls (WT), while E-Syt2 was detected in other tissues including heart (FIG. 8D). Primary lymphocytes derived from mice with targeted disruption of BCL70, MALT7 or CARMA7 revealed that all 3 proteins are essential for adaptive immunity and specifically required to mediate NF-κB activation after antigen receptors triggering. In search for immune defects in E-Syt2-deficient mice, we first examined lymphocyte differentiation in E-Syt2^{-/-} and conditional knockout mice. Although these mice displayed normal numbers of thymocytes compared to control mice, total splenic and lymph nodes cellularity was significantly reduced compared to wild-type mice (FIGS. 6A, 6B and 6K, 6L). There was no appreciable difference in the percentages of CD4+ and CD8+ thymocytes although a reduction of CD25+Foxp3+ regulatory T cells was evident (FIG. 6M, 6N). In contrast, we observed a marked decrease in all CD3+ T cell subsets (CD4+, CD8+, CD25+Foxp3+ cells) in the spleen and lymph nodes of E-Syt2′⁻′ and conditional knock-out mice compared to control mice (FIGS. 6C, E, G and 6M, N, O). The number of CD19⁺ B lymphocytes was also significantly decreased in the lymph nodes (Figure S6C). Interestingly, the absence of E-Syt2 impacted strongly on CD44+CD62L+ memory T cell homeostasis (FIG. 6F), as less activated CD4+ and memory T cells are present in the spleen and lymph nodes of E-Syt2-deficient mice. Apoptosis of CD4⁺ lymphocytes purified from E-Syt2^{-/-} mice and stimulated for 3 days with anti-CD3 and anti-CD28 coated beads was consistently increased compared to CD4⁺ lymphocytes purified from control mice (FIGS. 6H and I). This effect was strongly reduced after addition of exogenous IL-2 to the culture medium. Moreover, we observed a decrease in the level of secreted IL-2 in CD4+ T cells isolated from E-Syt2^{-/-} mice and stimulated for 24 hours with anti-CD3 and anti-CD28 or phorbol 12-myristate 13-acetate (PMA) and ionomycin, compared to CD4⁺ cells purified from control mice (FIG. 6J). These results suggest that failure to induce IL-2 in response to TCR stimulation contributes to abnormal maintenance of E-Syt2-deficient CD4⁺ lymphocytes.

### Example 8: Development and Selection of T Cells in E-Syt2−/− Mice

T cell development and the activation of naïve T-cells are dependent on the signal produced by the engagement of the TCR with the peptide-MHC complex presented by the antigen presenting cells (APC) and the activation of different signaling pathways including NF-κB, mediate the expression of numerous genes involved in T-cell activation.

Naive CD4+ T cell proliferate and differentiate into effector T cells in response to TCR stimulation and produce cytokines such as IL-2. In addition, some of these effector T cells differentiate in memory T cells.

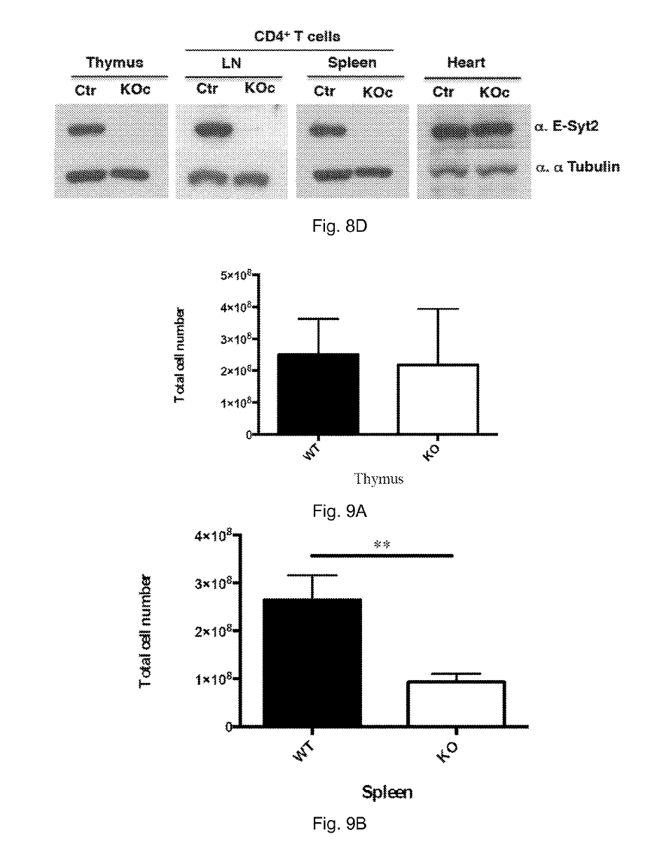

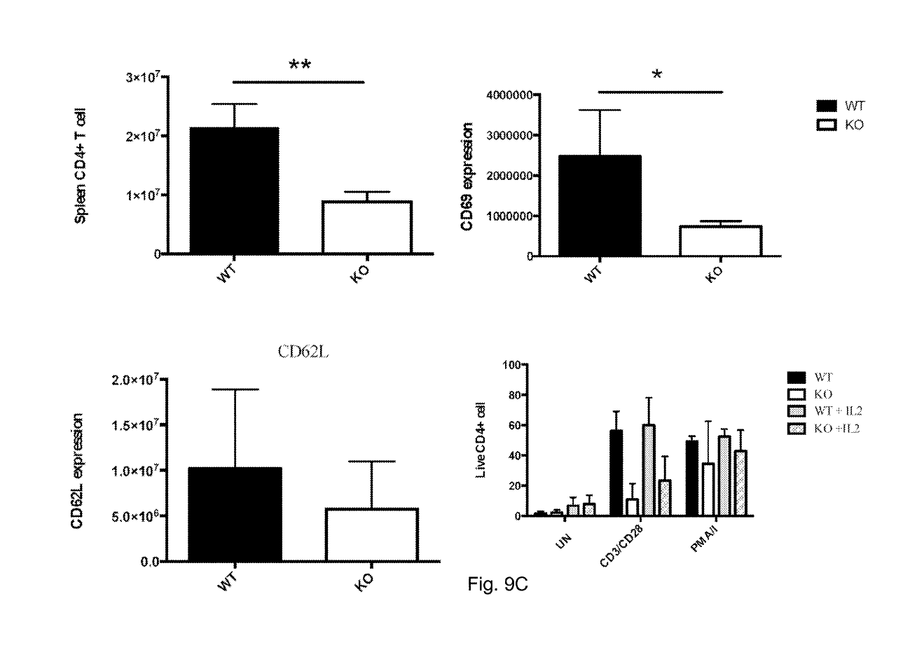

The total number of thymocytes was normal in both E-Syt2^{-/+} and E-Syt2^{-/-} mice compared to E-Syt2^{+/+} mice (FIG. 9a). Quantification of thymocytes subpopulations in thymus showed that double-negative (DN) cell numbers were relatively comparable in these mice.

However, the total numbers of splenocytes (FIG. 9b) and in particular of CD4+ and CD8+ T cells in the spleen of E-Syt2^{-/+} and E-Syt2^{-/-} mice was reduced compared to wild-type mice (FIG. 9c) and surface expression levels of CD3, CD4, CD8, CD25, CD44 was also comparable. Moreover, we consistently observed a decrease in the number of activated T cells in the spleen and lymph nodes of E-Syt2^{-/-} mice as detected by the expression analyses of the cell surface marker CD69 (marker of activated CD4+ T cells). These results were consistent with a previous report showing that ESYT2 was identified in a large-scale screening searching for dominant effectors affecting CD69 expression in response to T-cell activation³⁹. The expression of CD62L, a marker of memory T cells, was also reduced in CD4+ T cells from E-Syt2^{-/+} and E-Syt2^{-/-} mice compared to wild-type mice. These results suggest that both activated CD4+ cells and memory cells are less expressed in the spleen.

### Example 9: E-Syt2 is Required for the Survival of CD4+ Lymphocytes

Since E-Syt2 is a novel interacting protein of CARMA1 that plays a pivotal role in TCR-mediated NF-κB activation, we sought to determine whether T-cell proliferation and survival were altered in its absence, using T cells isolated from both control and ESYT2^{-/-} mice. CD4+ T lymphocytes were purified from spleen and lymph nodes and their proliferation was measured after stimulation with beads coated with anti-CD3 and anti-CD28 antibodies or upon treatment with phorbol myristate acetate and lonomycin (FIG. 9e).

The proliferation of CD4+ T lymphocytes was measure by flow cytometry using Carboxyfluorescein succinimidyl ester (CSFE). These experiments showed that the loss of E-Syt2 did not affect CD4+ T lymphocyte proliferation. However, the viability of CD4+ lymphocytes purified from E-Syt2^{-/-} mice and stimulated for 4 days with anti-CD3 and anti-CD28 coated beads was consistently reduced compared to CD4+ lymphocytes purified from control mice. Interestingly, this effect was reversed by addition of exogenous IL-2 to the culture medium. Moreover preliminary data indicated a decrease in the level of secreted interleukin-2 (IL-2) in E-Syt2^{FLN/FLN} mice compared to control mice, after 16 hours of stimulation with anti-CD3 and anti-CD28 coated beads. Altogether, these results indicate that the failure to induce IL-2 in response to TCR stimulation contributes to the decreased viability of E-Syt2-deficient CD4+ lymphocytes. Moreover we also observe a decrease in the level of secreted interleukin-2 (IL-2) in E-Syt2^{-/-} mice compared to control mice, after 16 hours of stimulation with anti-CD3 and anti-CD28 coated beads. Altogether, these results indicate that the failure to induce IL-2 in response to TCR stimulation contributes to the decreased viability of E-Syt2-deficient CD4+ lymphocytes.

### Example 10: E-Syt2 is Required for TCR-Induced NF-κB Activation

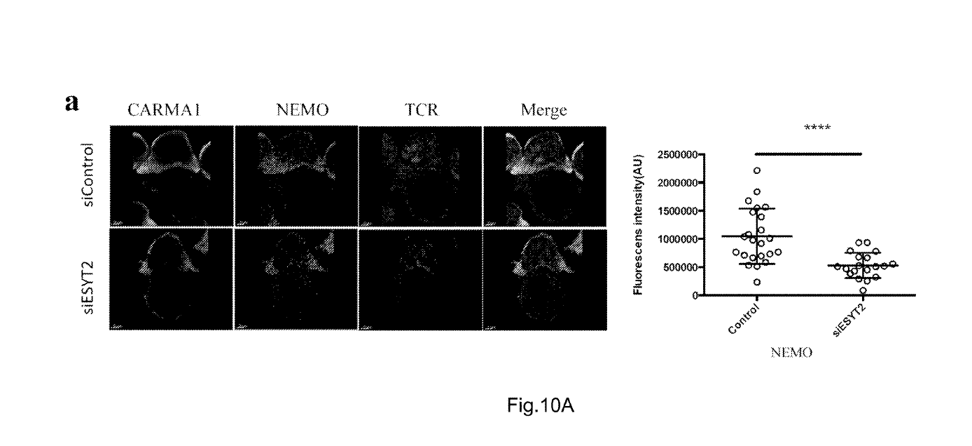

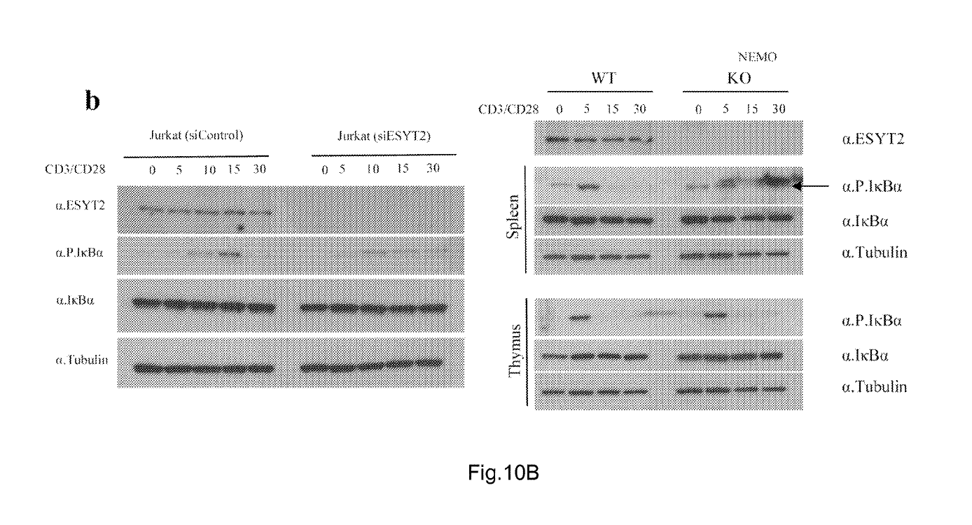

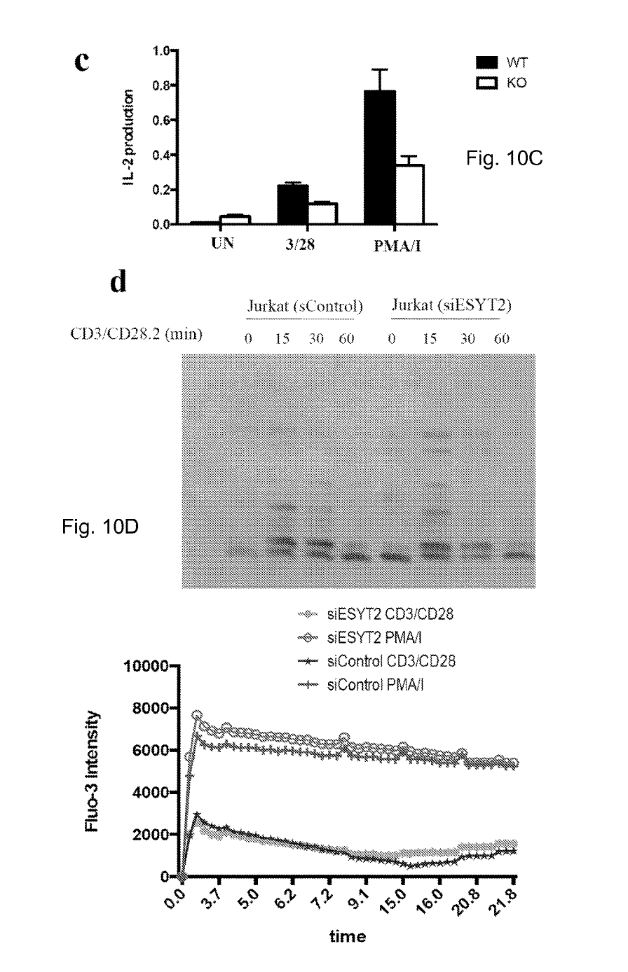

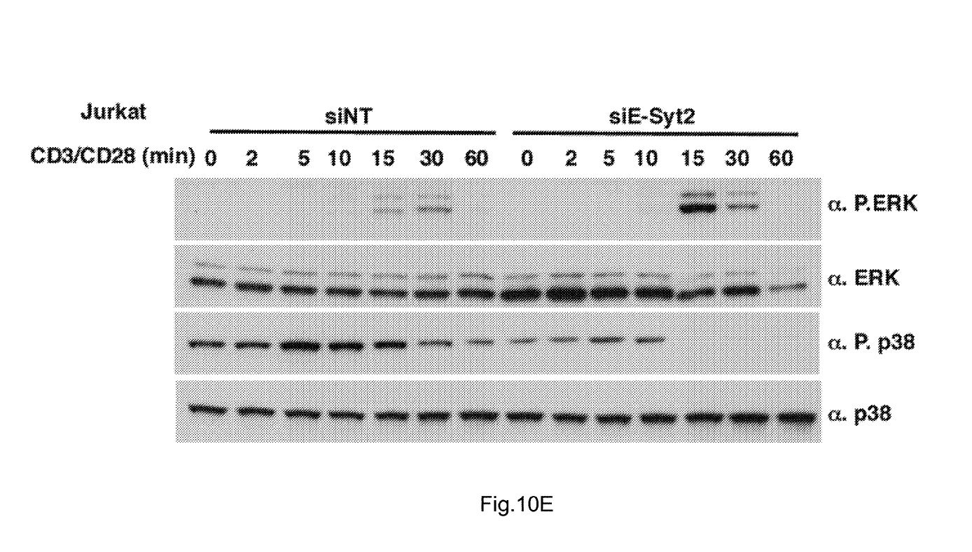

Previous studies showed that following TCR engagement, the IKK complex regulatory subunit NEMO localized to the immunological synapse²⁹. This event likely requires CARMA1²⁸. Since CARMA1 is not recruited to the immunological synapse in ESYT2-depleted cells, we examined the presence of NEMO at this location. Consistently, we observed a strong decrease of NEMO at the immunological synapse of ESYT2-depleted Jurkat cells (FIG. 10a).

The recruitment of the CBM complex to the IS in response to TCR stimulation is required for NF-κB activation. Since we demonstrated that ESYT2 allows the localization of the CBM complex to the IS, we hypothesized that ESYT2 might be a new component of the TCR-mediated NF-κB pathway. In order to investigate this hypothesis, we investigate NF-κB activation in ESYT2-depleted Jurkat cells. Upon TCR stimulation, IKK complex is activated and triggers the phosphorylation and degradation of the NF-κB inhibitory protein, IκBα. NF-κB is then translocated to the nucleus and activates the expression of its target genes. To investigate the status of NF-κB activation in E-Syt2-depleted Jurkat cells, we monitored IκBα degradation at different time points following TCR stimulation (FIG. 10b). Western blot analysis of control and ESYT2 depleted cells showed that the phosphorylation of IκBα was less intense in ESYT2-depleted cells than in control cells. Moreover, the phosphorylation of IκBα disappeared after 30 minutes of stimulation in control cells unlike in E-Syt2-depleted cells. Compared to control cells the weak phosphorylation of IκBα in ESYT2-depleted cells remained for a longer time, demonstrating that the degradation of IκBα is impaired in ESYT2-depleted Jurkat cells. These results suggest that ESYT2 regulates the activation of NF-κB pathway in Jurkat cells, upon TCR stimulation. Analysis of E-Syt2-depleted Jurkat cells suggested that E-Syt2 is implicated in the activation of the NF-κB pathway, upon CD3 and CD28 stimulation. To confirm these results we monitored IκBα phosphorylation by western blot in E-Syt2-deficient CD4+ lymphocytes purified from spleen and thymus and stimulated with anti-CD3 and anti-CD28 (FIG. 10b, right panel). The results we obtained with these cells corroborate those found in ESYT2-depleted Jurkat cells. Indeed, we observe a strong phosphorylation of IκBα in CD4+ cells extracted from E-Syt2^{+/+} after 5 minutes of CD3/CD28 stimulation that contrast with the weak phosphorylation observed in ESYT2-deficient CD4+ cells. Interestingly, no differences were found in the levels of phosphorylation of IκBα in naïve CD4+ cells purified from the thymus.

In contrast, ESYT2-depletion had no effect on the level of tyrosine phosphorylation, monitored by western blot and on Ca2+ influx (FIG. 10d). To decipher the molecular mechanism responsible for the defective T cell functions in the absence of E-Syt2, the signaling pathways activated following TCR engagement were analyzed. The calcium influx induced either by TCR stimulation or PMA/ionomycine treatment, and the overall levels of Tyrosine-phosphorylated proteins were of similar magnitude in E-Syt2-depleted Jurkat cells compared to control cells (FIG. 10d). In addition, stimulation of E-Syt2-depleted Jurkat cells with anti-CD3 and anti-CD28 antibodies resulted in a marked reduction of p38 phosphorylation while ERK phosphorylation was enhanced (FIG. 10e).

### Example 11: E-Syt2 Tethers the ER at the IS but is Dispensable for the Recruitment of Ubiquitinated BCL10 at the ER

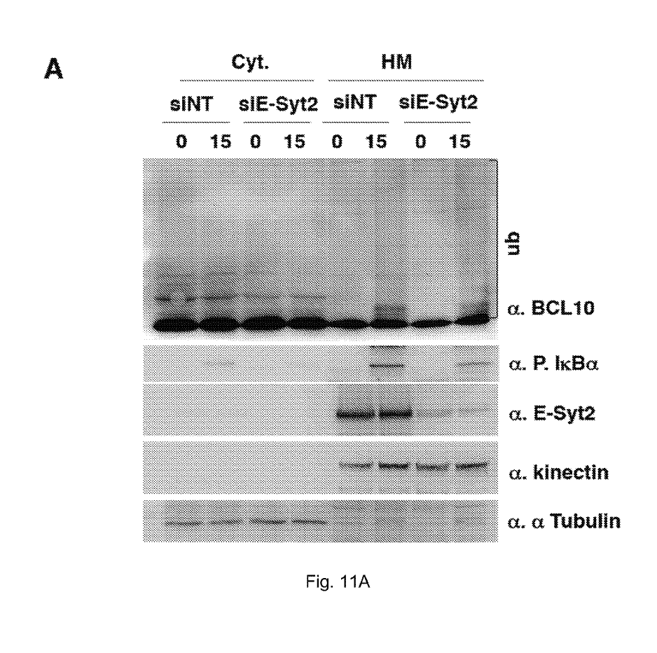

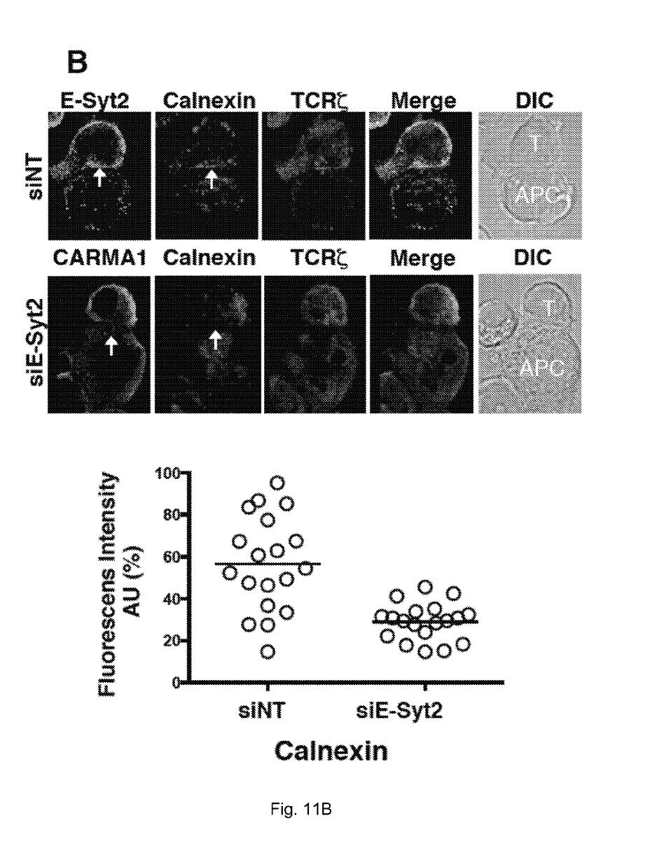

It has been recently shown that the ER acts as a platform for ubiquitinated components of the TCR-mediated NF-κB pathway and that their recruitment to the ER is dependent on CARMA1. Since E-Syt2 mediates contacts between the ER and the plasma membrane, we hypothesized that E-Syt2 could play the same tethering function at the IS in order to recruit ER-resident ubiquitinated signaling molecules. By cell fractionation of Jurkat cells (FIG. 11A) and confocal microscopy (FIG. 11B), we observed that E-Syt2 accumulates in heavy membrane fractions (HM), which contains ER, and colocalizes with Calnexin, an integral protein of the ER. Interestingly, E-Syt2 depletion decreased ER localization to the IS (FIG. 11B), but was dispensable for the localization of ubiquitinated BCL10 in HM (FIG. 11A), indicating that E-Syt2 plays its tethering function at the IS but is dispensable for the recruitment of ubiquitinated BCL10 to the ER.

### Example 12: Analysis of E-Syt2 Conditional Deficient Mice

The development and function of T cells is analyzed and evaluated in the E-SYT2 conditional deficient mice. The total number of thymocytes in homozygote and heterozygote mice is evaluated and compared to wild type mice. The expression of CD4 and CD8 surface markers is evaluated by flow cytometry, and double positive (DP) and double negative (DN) thymocytes are quantified. The expression of CD25 and CD44 in DN thymocytes reflects the different stages of gene rearrangement in the TCR: CD25−CD44+, CD25+CD44+, CD25+CD44−, and CD25−CD44−.

The number of cells in lymph nodes in homozygote, heterozygote, and wild type mice is also evaluated. The response of mature T cell lymphocytes in these mice to different stimuli is compared by flow cytometry, and the expression of CD25, CD44, CD69 markers is measured after stimulation of these cells with anti-CD3 and anti-CD28 antibodies or phorbol ester (PMA) and Ionomycin.

The proliferation of peripheral T cells is measured after stimulation, and any corrections in defects in cell proliferation in E-Syt2 knock-out mice after the addition of exogenous Interleukin-2 (IL-2) are evaluated. The activation of NF-κB and IL-2 production is evaluated in CD4+ T cell lymphocytes selected by negative selection.

Other signaling pathways that come into play by TCR stimulations (Erk1, Erk2, Akt and p38, and then Jnk and PKC-θ activation) are also analyzed by Western Blot.

### Example 13: Identification of Potential Alterations of E-Syt2 Gene in ABC-DLBCL

The activated B cell-like diffuse large B cell lymphoma (ABC-DLBCL) is characterized by chronic active B-cell receptor signaling and constitutive activation of the NF-κB pathway linked to mutations in various genes regulating NF-κB, such as activating mutations of CARMA1. For these reasons, we believe that E-Syt2 could also be critical for the survival of ABC-DLBCL. To address this question, a E-Syt2 shRNA is cloned into a GFP-expressing retroviral vector, and cell lines are infected representing ABC-DLBCL (OCI-Ly3, OCI-Ly10, U2932, RIVA, SU-DHL-2). The germinal-center B-cell-like (GCB) subtypes (BJAB, OCI-Ly19, SU-DHL-4 et SU-DHL-6), the proliferation of which is independent of NF-κB, are used as a negative control. We expect that if E-Syt2 is required for ABC-DLBCL survival, the fraction of GFP-positive, shRNA-expressing cells will decrease over time in ABC-DLBCL, but not in GCB-DLBCL. shRNAs targeting IKKβ, CARMA1, BCL10 and MALT1, are used as controls. We will next test whether the lethality of an shRNA targeting E-Syt2 could be prevented by re-expression of wild-type E-Syt2 or of a mutant impaired in its association with CARMA1 (E-Syt2Δ530-680). In the case E-Syt2 turns out to be required for survival of these tumor cells, therapeutic molecules that specifically target E-Syt2 to prevent the constitutive activation of NF-κB signaling as a treatment of ABC-DLBCL lymphomas are designed/identified.

### Example 14: Spontaneous Dermatitis in Aging E-Syt2^{-/-} and E-Syt2^{-/+} Mice

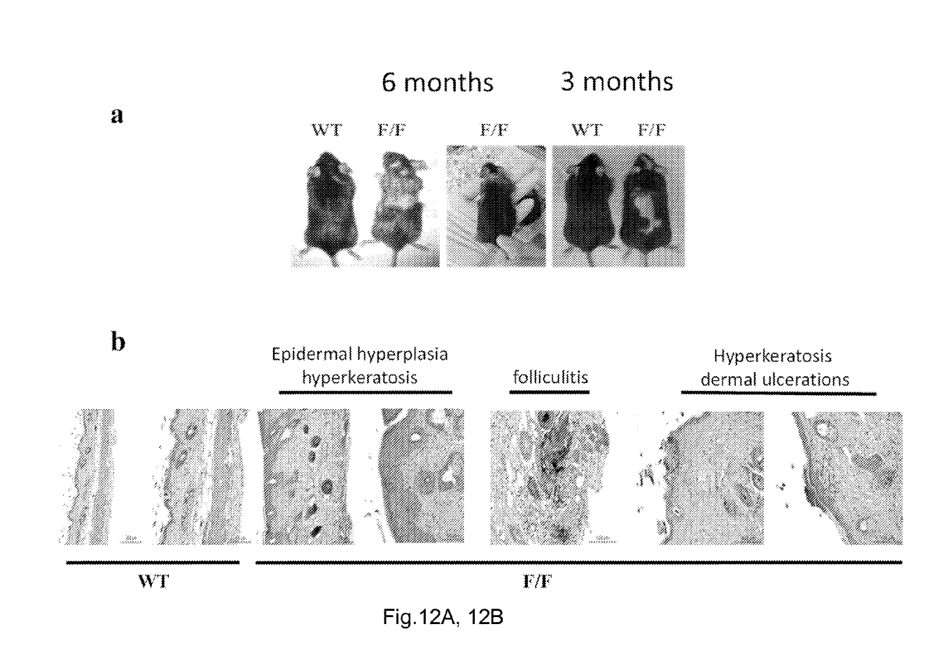

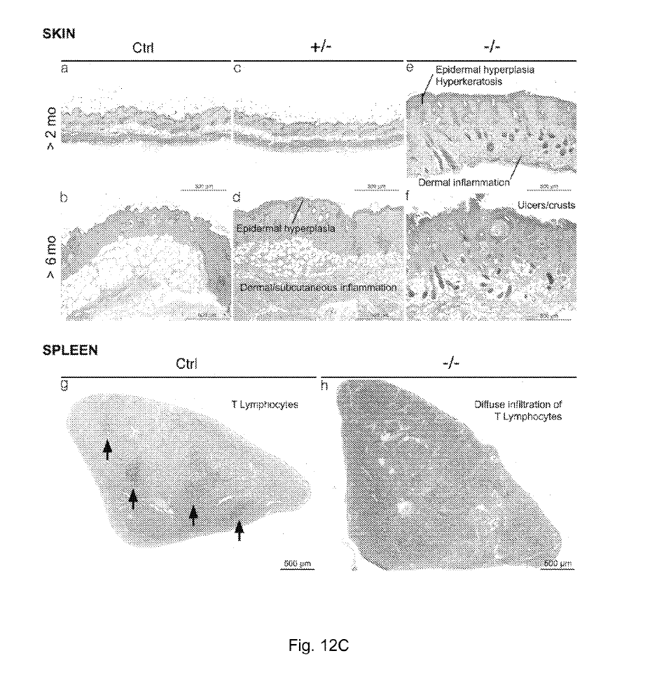

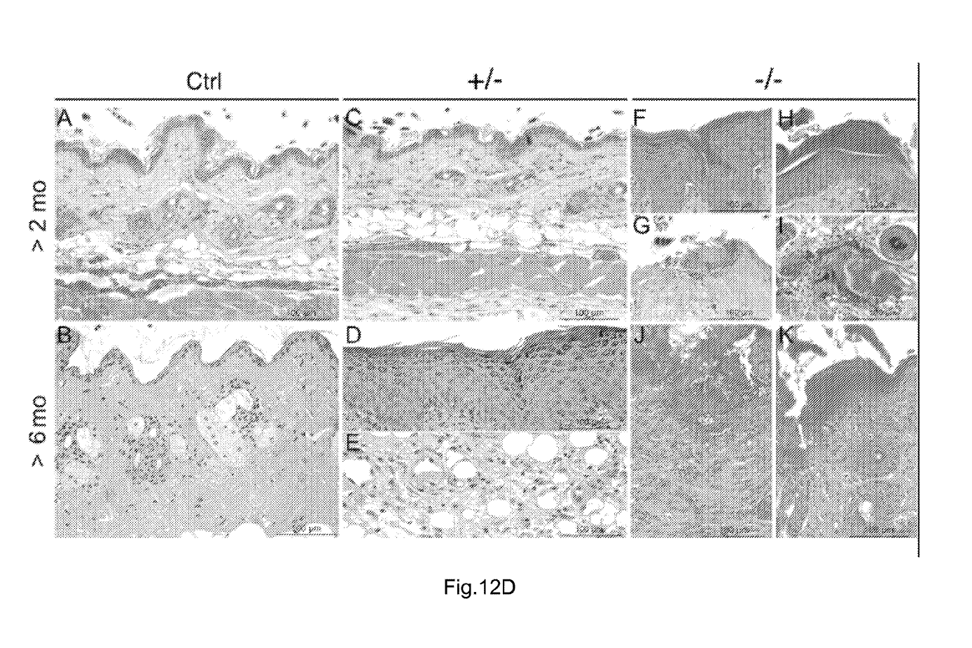

E-Syt2^{-/-} and E-Syt2^{-/+} mice seemed to be overly normal at birth and their growth and development were indistinguishable from those of their wild-type E-Syt2^{+/+} littermates. After 14 weeks following their birth, we noted that the majority of these mice developed dermatitis (FIG. 12A, B), especially developed psoriasiform skin lesions with age. Histological analysis of the skin from hetero- and homozygous mice showed that skin samples from various sites in their body was characterized by marked acanthosis (epidermal hyperplasia) and hyperkeratosis (ortho or parakiratosis) (FIG. 12A, B). In addition, a dermal ulceration and folliculitis were apparent in these skin samples (FIG. 12A, B). More particularly, the lesions appeared first in young (>2 months) homozygous mice, and were more severe in older mice (>6 months). These lesions were characterized by multifocal acanthosis (epidermal hyperplasia), hyperkeratosis, fibrinous crust formation and epidermal ulceration (FIG. 12C). Moreover, histopathological and immunohistochemical analyses of the spleen in the same mice detected an alteration of the T cell organization in homozygous mice; T cells were indeed observed diffusely in the spleen and not limited to the periarteriolar lymphoid sheaths (PALS) as in control mice (FIG. 12C) suggesting dysregulated immune activation.

Atopic dermatitis is a common clinical feature shared by mice harboring defects in the NF-κB pathway, including a point mutation of CARMA1 (“unmodulated” mice); this clinical phenotype is recapitulated in E-Syt2^{-/-} mice.

### DISCUSSION

We searched for a molecule that contributes to NF-κB signaling by recruiting CARMA1 to the IS through its SH3 domain. We found that ESYT2 met these criteria and we provide evidence that ESYT2 physically interacts with CARMA1, colocalizes with it and mediates the recruitment of CARMA1, PKCθ, BCL10, ADAP, NEMO and the ER to the IS. Not only does ESYT2 play a critical function for organizing the localization of critical signaling molecules in the IS but also in TCR MCs.

Notably, the TCR and BCR share analogous signaling pathways including the one required for NF-κB activation′. Thus, on the basis of these similarities, ESYT2 might also play an important proximal function in B cells since antibody-producing B cells and natural killer cells also form IS with APC and target cells respectively^{56,51}; however, its expression in B cells in barely reduced compared to T cells. In addition, it has been shown by many groups, that CARMA1 is phosphorylated by PKCθ and PKCβ in at least three serine residues, Ser552, Ser637 and Ser645, in human CARMA1^{40,41}. These serine are contained in the linker sequence between the coiled coil domain and the PDZ domains of CARMA1, which control a conformational switch from an inactive to an active state^{40,41} Although we have shown that PKCθ controls the interaction between CARMA1 and ESYT2, PKCβ might play a similar function to ensure BCR-mediated B cell survival. The presence of SH3 domain in all MAGUKs opens the possibility that ESYT2 could target other MAGUK from the cytosol to the plasma membrane. These domains present important functions in the MAGUK superfamily. Consistently, a point mutation in the SH3 domain of discs large-1 (dlg; a drosophila homolog of postsynaptic density-95 (PSD-95)) causes a loss of septate junctions and overproliferation in the imaginal discs⁵². The PSD-95, ZO-1 and ZO-3 structures suggest that their SH3 domains have an unusually extended fold composed of six (3 strands rather than five, the last strands being contained on the Guk domain fragment⁵³⁻⁵⁵. The SH3 and Guk domains are separated by a short flexible amino acid stretch, called the Hook domain, and interact with each other. Additionally, the PDZ3 domains of ZO-1 and CARMA family MAGUK also extensively interact with their SH3 domains to form integral structural units⁵⁶. The intramolecular interaction between the GUK and SH3 domains is prevented by the interaction of a ligand with the linker domain, which instead induces extramolecular interactions with another MAGUK protein and the PDZ3 domain exert an allosteric regulation between the PDZ3 and SH3-Guk^{55,57}. It remains to be determined whether an interaction of the SH3 domain could also affect the function of the MAGUK domain and whether ESYT2 binding to the SH3 domain of CARMA1 may prevent intramolecular interactions for the benefit of extramolecular interactions.

Our results indicate that PKCθ is recruited to the IS independently from CARMA1 while both depend on ESYT2 for being recruited their. In addition, ESYT2 associates with CARMA1 in a signal dependent manner probably in the IS since it has been shown that CARMA1 activation takes place there²⁸. Altogether, this suggests that ESYT2 might enable the activation of CARMA1 by PKCθ at the IS. In contrast, the kinesin protein GAKIN interacts with CARMA1, decreases its redistribution to the IS and attenuates TCR signaling to NF-κB⁵⁸. The mechanism by which ESYT2 recruits PKCθ to the IS remains to be determined. Different studies have shed some light on how PKCθ is recruited to the IS and is activated. Kong and colleagues have shown that the polyproline motif in the V3 domain of PKCθ presents a critical role for the recruitment of PKCθ to the IS through its Lck-mediated interaction with CD28⁸. The C1 domain of PKCθ has also been shown to localize transiently to the IS⁵⁹. Hence, ESYT2 may act as a bridge between CD28 and PKCθ or may target the C1 domain to allow the recruitment of PKCθ to the IS with high stoichiometry.

Furthermore, BCL10, NEMO and ADAP might be indirectly recruited to the IS since BCL10 and ADAP interact respectively with the CARD domain and C-terminal region of CARMA1, which is also essential for the recruitment of the IKK to the IS^{13,28,60}.

E-Syt2 has been proposed to tether the ER to the plasma membrane and to mediate the transport of lipids through the presence of a synaptotagmin-like mitochondrial-lipid-binding protein (SMP) domain^{35,61}.

An interesting hypothesis for the function of ESYT proteins is that they may promote ER-PM contact sites to enable calcium flux and exchange of lipids between the two bilayers. ESYTs do not seem to play a role in calcium flux because siRNA knockdown of all three ESYTs does not have any effect on Ca2+ flux³⁵. In addition our experiments indicate that ESYT2 knock down does not impair TCR or PMA-induced Ca2+ flux in T cells. However, ESYT2 might be important for the local concentration of Ca2+ in the IS and this could be evaluated using imaging techniques and fluorescent Ca2+ biosensors⁶². Besides Ca2+, lipids play an important role for the function of T lymphocytes⁶³ and lipid rafts have been proposed as mediators of protein clustering at the IS. TCR-induced PIP2-hydrolysis by phospholipases such as PLCγ generates lipid diacylglycerol (DAG) and inositol (1, 4, 5) triphophosphate (IP3), which are essential second messengers in T cells. PIP2 are also important second messengers, which bind to signaling proteins containing a pleckstrin homology domain (PH). Their phosphorylation mediated by PI3K generates phosphatidylinositol (3, 4, 5) triphosphate. PKCs are lipid-sensitive enzymes that are activated by agonists that increase DAG or by PMA. Furthermore, nPKC such as PKCθ binds DAG with high affinity compared to cPKC without a Ca2+ requirement⁶⁴. Phosphorylation of the activation loop of PKCθ is required for its activity⁶⁵ and two different kinases, PDK1 and GLK, have been proposed to play this role^{66,67}.

Since ESYT2 mediates the transport of lipids and the recruitment of PKCθ and CARMA1 to the IS, we made the hypothesis that ESYT2 could transfer membrane lipids to PKCθ in order to activate CARMA1. Consistent with this idea, we observed that ESYT2 enhanced PKCθ activation by liposomes depending of their lipid composition to phosphorylate CARMA1. We took also advantage of the use of fluorescent phorbol esters to confirm that ESYT2 increases the transfer of lipids from ESYT2 to PKCθ. Our results thus suggest that ESYT2 serves as a platform for the activation of CARMA1 by PKCθ and the subsequent activation of NE-KB. Deletion of ESYT2 SMP domain might not interfere with the recruitment of PKCθ to the IS but might prevent its activation and consequently downstream signaling leading to NF-κB activation. Interestingly, in addition of recruiting PKCθ to the IS, ESYT2 mediates the recruitment of PAK1 to clathrin coated pit allowing endocytosis of activated FGFR1³⁸. PAK1 shares certain biological properties with PKCθ. Both interacts with phospholipids, are activated by lipids and are dynamically recruited to the IS^{68,69}. It has been shown that phosphoinositides promotes PAK1 activation in synergy with Rac1⁶⁸. However, while ESYT2 increases PKCθ activation, it inhibits the activation of PAK1 by the GTPases Cdc42 and Rac³⁸. Future studies will determine whether ESYT2 is required for the localization of PAK1 to the IS and whether it transfer lipids to PAK1. In particular, the generation of a mice model carrying a loss of function mutation of ESYT2 SMP domain will be a major breakthrough for understanding how ESYT2 regulate these kinases.

Importantly, mutations of the key components of the antigen-mediated signaling pathways are associated with constitutive CBM-mediated signaling, and with the development of particular subtypes of human B-cell lymphomas, including mucosa-associated lymphoid tissue (MALT) and diffuse large B-cell (DLBCL) lymphomas, which represent 30% of non-Hodgkin lymphomas. Constitutive anti-apoptotic NF-κB signaling is a hallmark of ABC-DLBCL. Indeed, approximately 10% of ABC-DLBCLs harbors activating missense mutations localized within the coiled-coil domain of CARMA1 and expression of these mutants leads to constitutive and enhanced antigen receptor-dependent activation of NF-κB. In contrast to CARMA1, MALT1 is not mutated or translocated in ABC-DLBCL, but ABC-DLBCL cell lines are dependent on its catalytic activity for survival. These results further suggest that MALT1 protease activity is a potential target for pharmacological treatment of ABC-DLBCL and diverse MALT1 inhibitors are currently being tested for treatment of ABC-DLBCL. ESYT2 is with MALT1 the only known enzyme in the antigen-mediated cascade and inhibiting. Therefore, it is a hypothesis of this invention that inhibiting its activity is useful for the treatment of this malignancy.

### REFERENCES

1. Messali, N., Génin, P. & R, W. Deregulated antigen-induced NF-kB signalling pathway in the development of human B-cells lymphomas. journal of Leukemia in press 1, 105 (2013).
2. Rosse, C. et al. PKC and the control of localized signal dynamics. Nat Rev Mol Cell Biol 11, 103-112 (2010).
3. Ron, D. & Kazanietz, M. G. New insights into the regulation of protein kinase C and novel phorbol ester receptors. FASEB J 13, 1658-1676 (1999).
4. Liu, Y. et al. Regulation of protein kinase Ctheta function during T cell activation by Lck-mediated tyrosine phosphorylation. J Biol Chem 275, 3603-3609 (2000).
5. Bi, K. & Altman, A. Membrane lipid microdomains and the role of PKCtheta in T cell activation. Semin Immunol 13, 139-146 (2001).
6. Tavano, R. et al. CD28 and lipid rafts coordinate recruitment of Lck to the immunological synapse of human T lymphocytes. J Immunol 173, 5392-5397 (2004).
7. Hofinger, E. & Sticht, H. Multiple modes of interaction between Lck and CD28. J Immunol 174, 3839-3840; author reply 3840 (2005).
8. Kong, K. F. et al. A motif in the V3 domain of the kinase PKC-theta determines its localization in the immunological synapse and functions in T cells via association with CD28. Nat Immunol 12, 1105-1112 (2011).
9. Messali, N., Génin, P. & R, W. Deregulated antigen-induced NF-kB signalling pathway in the development of human B-cells lymphomas. journal of Leukemia, in press (2013).
10. Wu, C. J. & Ashwell, J. D. NEMO recognition of ubiquitinated Bc110 is required for T cell receptor-mediated NF-kappaB activation. Proc Natl Acad Sci USA 105, 3023-3028 (2008).
11. Oeckinghaus, A. et al. Malt1 ubiquitination triggers NF-kappaB signaling upon T-cell activation. EMBO J 26, 4634-4645 (2007).
12. Shambharkar, P. B. et al. Phosphorylation and ubiquitination of the IkappaB kinase complex by two distinct signaling pathways. EMBO J 26, 1794-1805 (2007).
13. Medeiros, R. B. et al. Regulation of NF-kappaB activation in T cells via association of the adapter proteins ADAP and CARMA1. Science 316, 754-758 (2007).
14. Srivastava, R., Burbach, B. J. & Shimizu, Y. NF-kappaB activation in T cells requires discrete control of IkappaB kinase alpha/beta (IKKalpha/beta) phosphorylation and IKKgamma ubiquitination by the ADAP adapter protein. J Biol Chem 285, 11100-11105 (2010).
15. Srivastava, R., Burbach, B. J., Mitchell, J. S., Pagan, A. J. & Shimizu, Y. ADAP regulates cell cycle progression of T cells via control of cyclin E and Cdk2 expression through two distinct CARMA1-dependent signaling pathways. Mol Cell Biol 32, 1908-1917 (2012).
16. Su, H. et al. Requirement for caspase-8 in NF-kappaB activation by antigen receptor. Science 307, 1465-1468 (2005).
17. Rebeaud, F. et al. The proteolytic activity of the paracaspase MALT1 is key in T cell activation. Nat Immunol 9, 272-281 (2008).
18. Coornaert, B. et al. T cell antigen receptor stimulation induces MALT1 paracaspase-mediated cleavage of the NF-kappaB inhibitor A20. Nat Immunol 9, 263-271 (2008).
19. Pelzer, C. et al. The protease activity of the paracaspase MALT1 is controlled by monoubiquitination. Nat Immunol 14, 337-345 (2013).
20. Egawa, T. et al. Requirement for CARMA1 in antigen receptor-induced NF-kappa B activation and lymphocyte proliferation. Curr Biol 13, 1252-1258 (2003).
21. Ruland, J. et al. Bcl10 is a positive regulator of antigen receptor-induced activation of NF-kappaB and neural tube closure. Cell 104, 33-42 (2001).
22. Jun, J. E. et al. Identifying the MAGUK protein Carma-1 as a central regulator of humoral immune responses and atopy by genome-wide mouse mutagenesis. Immunity 18, 751-762 (2003).
23. Hara, H. et al. The MAGUK family protein CARD11 is essential for lymphocyte activation. Immunity 18, 763-775 (2003).
24. Ruland, J., Duncan, G. S., Wakeham, A. & Mak, T. W. Differential requirement for Malt1 in T and B cell antigen receptor signaling. Immunity 19, 749-758 (2003).
25. Sun, Z. et al. PKC-theta is required for TCR-induced NF-kappaB activation in mature but not immature T lymphocytes. Nature 404, 402-407 (2000).
26. Ruefli-Brasse, A. A., French, D. M. & Dixit, V. M. Regulation of NF-kappaB-dependent lymphocyte activation and development by paracaspase. Science 302, 1581-1584 (2003).
27. Wang, D. et al. CD3/CD28 costimulation-induced NF-kappaB activation is mediated by recruitment of protein kinase C-theta, Bc110, and IkappaB kinase beta to the immunological synapse through CARMA1. Mol Cell Biol 24, 164-171 (2004).
28. Hara, H. et al. The molecular adapter Carma1 controls entry of IkappaB kinase into the central immune synapse. J Exp Med 200, 1167-1177 (2004).
29. Weil, R. et al. Induction of the NF-kappaB cascade by recruitment of the scaffold molecule NEMO to the T cell receptor. Immunity 18, 13-26 (2003).
30. Bi, K. et al. Antigen-induced translocation of PKC-theta to membrane rafts is required for T cell activation. Nat Immunol 2, 556-563 (2001).
31. Huang, J. et al. CD28 plays a critical role in the segregation of PKC theta within the immunologic synapse. Proc Natl Acad Sci USA 99, 9369-9373 (2002).
32. Combs, J. et al. Recruitment of dynein to the Jurkat immunological synapse. Proc Natl Acad Sci USA 103, 14883-14888 (2006).
33. Min, S. W., Chang, W. P. & Sudhof, T. C. E-Syts, a family of membranous Ca2+-sensor proteins with multiple C2 domains. Proc Natl Acad Sci USA 104, 3823-3828 (2007).
34. Xu, J. et al. Structure and Ca(2)(+)-binding properties of the tandem C(2) domains of E-Syt2. Structure 22, 269-280 (2014).
35. Giordano, F. et al. PI(4,5)P(2)-dependent and Ca(2+)-regulated ER-PM interactions mediated by the extended synaptotagmins. Cell 153, 1494-1509 (2013).
36. Manford, A. G., Stefan, C. J., Yuan, H. L., Macgurn, J. A. & Emr, S. D. ER-to-plasma membrane tethering proteins regulate cell signaling and ER morphology. Dev Cell 23, 1129-1140 (2012).
37. Jean, S. et al. Extended-synaptotagmin-2 mediates FGF receptor endocytosis and ERK activation in vivo. Dev Cell 19, 426-439 (2010).
38. Jean, S., Tremblay, M. G., Herdman, C., Guillou, F. & Moss, T. The endocytic adapter E-Syt2 recruits the p21 GTPase activated kinase PAK1 to mediate actin dynamics and FGF signalling. Biol Open 1, 731-738 (2012).
39. Chu, P. et al. Systematic identification of regulatory proteins critical for T-cell activation. J Biol 2, 21 (2003).
40. Matsumoto, R. et al. Phosphorylation of CARMA1 plays a critical role in T Cell receptor-mediated NF-kappaB activation. Immunity 23, 575-585 (2005).
41. Sommer, K. et al. Phosphorylation of the CARMA1 linker controls NF-kappaB activation. Immunity 23, 561-574 (2005).
42. Roumier, A. et al. The membrane-microfilament linker ezrin is involved in the formation of the immunological synapse and in T cell activation. Immunity 15, 715-728 (2001).
43. Yokosuka, T. et al. Spatiotemporal regulation of T cell costimulation by TCR-CD28 microclusters and protein kinase C theta translocation. Immunity 29, 589-601 (2008).
44. Wang, D. et al. A requirement for CARMA1 in TCR-induced NF-kappa B activation. Nat Immunol 3, 830-835 (2002).
45. Hayashi, K. & Altman, A. Protein kinase C theta (PKCtheta): a key player in T cell life and death. Pharmacol Res 55, 537-544 (2007).
46. Weil, R. & Israel, A. Deciphering the pathway from the TCR to NF-kappaB. Cell Death Differ 13, 826-833 (2006).
47. Hirotsune, S. et al. Graded reduction of Pafah1b1 (Lis1) activity results in neuronal migration defects and early embryonic lethality. Nat Genet 19, 333-339 (1998).
48. Chen, L. et al. Gly369Cys mutation in mouse FGFR3 causes achondroplasia by affecting both chondrogenesis and osteogenesis. J Clin Invest 104, 1517-1525 (1999).
49. Meyers, E. N., Lewandoski, M. & Martin, G. R. An Fgf8 mutant allelic series generated by Cre- and Flp-mediated recombination. Nat Genet 18, 136-141 (1998).
50. Batista, F. D., Iber, D. & Neuberger, M. S. B cells acquire antigen from target cells after synapse formation. Nature 411, 489-494 (2001).
51. Bromley, S. K. et al. The immunological synapse. Annu Rev Immunol 19, 375-396 (2001).
52. Woods, D. F., Hough, C., Peel, D., Callaini, G. & Bryant, P. J. Dlg protein is required for junction structure, cell polarity, and proliferation control in Drosophila epithelia. J Cell Biol 134, 1469-1482 (1996).
53. Lye, M. F., Fanning, A. S., Su, Y., Anderson, J. M. & Lavie, A. Insights into regulated ligand binding sites from the structure of ZO-1 Src homology 3-guanylate kinase module. J Biol Chem 285, 13907-13917 (2001).
54. Tavares, G. A., Panepucci, E. H. & Brunger, A. T. Structural characterization of the intramolecular interaction between the SH3 and guanylate kinase domains of PSD-95. Mol Cell 8, 1313-1325 (2001).
55. McGee, A. W. et al. Structure of the SH3-guanylate kinase module from PSD-95 suggests a mechanism for regulated assembly of MAGUK scaffolding proteins. Mol Cell 8, 1291-1301 (2001).
56. Pan, L., Chen, J., Yu, J., Yu, H. & Zhang, M. The structure of the PDZ3-SH3-GuK tandem of ZO-1 protein suggests a supramodular organization of the membrane-associated guanylate kinase (MAGUK) family scaffold protein core. J Biol Chem 286, 40069-40074 (2011).
57. Newman, R. A. & Prehoda, K. E. Intramolecular interactions between the SRC homology 3 and guanylate kinase domains of discs large regulate its function in asymmetric cell division. J Biol Chem 284, 12924-12932 (2009).
58. Lamason, R. L., Kupfer, A. & Pomerantz, J. L. The dynamic distribution of CARD11 at the immunological synapse is regulated by the inhibitory kinesin GAKIN. Mol Cell 40, 798-809.
59. Spitaler, M., Emslie, E., Wood, C. D. & Cantrell, D. Diacylglycerol and protein kinase D localization during T lymphocyte activation. Immunity 24, 535-546 (2006).
60. Gaide, O. et al. Carma1, a CARD-containing binding partner of Bc110, induces Bc110 phosphorylation and NF-kappaB activation. FEBS Lett 496, 121-127 (2001).
61. Schauder, C. M. et al. Structure of a lipid-bound extended synaptotagmin indicates a role in lipid transfer. Nature 510, 552-555 (2014).
62. Randriamampita, C. & Lellouch, A. C. Imaging early signaling events in T lymphocytes with fluorescent biosensors. Biotechnol J 9, 203-212 (2014).
63. Huang, Y. H. & Sauer, K. Lipid signaling in T-cell development and function. Cold Spring Harb Perspect Biol 2, a002428 (2010).
64. Giorgione, J. R., Lin, J. H., McCammon, J. A. & Newton, A. C. Increased membrane affinity of the C1 domain of protein kinase Cdelta compensates for the lack of involvement of its C2 domain in membrane recruitment. J Biol Chem 281, 1660-1669 (2006).
65. Liu, Y., Graham, C., Li, A., Fisher, R. J. & Shaw, S. Phosphorylation of the protein kinase C-theta activation loop and hydrophobic motif regulates its kinase activity, but only activation loop phosphorylation is critical to in vivo nuclear-factor-kappaB induction. Biochem J 361, 255-265 (2002).
66. Park, S. G. et al. The kinase PDK1 integrates T cell antigen receptor and CD28 coreceptor signaling to induce NF-kappaB and activate T cells. Nat Immunol 10, 158-166 (2009).
67. Chuang, H. C. et al. The kinase GLK controls autoimmunity and NF-kappaB signaling by activating the kinase PKC-theta in T cells. Nat Immunol 12, 1113-1118 (2011).
68. Strochlic, T. I., Viaud, J., Rennefahrt, U. E., Anastassiadis, T. & Peterson, J. R. Phosphoinositides are essential coactivators for p21-activated kinase 1. Mol Cell 40, 493-500 (2010).
69. Phee, H., Abraham, R. T. & Weiss, A. Dynamic recruitment of PAK1 to the immunological synapse is mediated by PIX independently of SLP-76 and Vav1. Nat Immunol 6, 608-617 (2005).
70. Weiss, A. & Littman, D. R. Signal transduction by lymphocyte antigen receptors. Cell 76, 263-274 (1994).
71. Boise, L. H. et al. CD28 costimulation can promote T cell survival by enhancing the expression of Bc1-XL. Immunity 3, 87-98 (1995).
72. Radvanyi, L. G. et al. CD28 costimulation inhibits TCR-induced apoptosis during a primary T cell response. J Immunol 156, 1788-1798 (1996).
73. Barouch-Bentov, R. et al. Protein kinase C-theta is an early survival factor required for differentiation of effector CD8+ T cells. J Immunol 175, 5126-5134 (2005).
74. Manicassamy, S., Gupta, S., Huang, Z. & Sun, Z. Protein kinase C-theta-mediated signals enhance CD4+ T cell survival by up-regulating Bcl-xL. J Immunol 176, 6709-6716 (2006).
75. Liang, Y. et al. The lymphoid lineage-specific actin-uncapping protein Rltpr is essential for costimulation via CD28 and the development of regulatory T cells. Nat Immunol 14, 858-866 (2013).
76. Shin, H. M. et al. NOTCH1 Can Initiate NF-kappaB Activation via Cytosolic Interactions with Components of the T Cell Signalosome. Front Immunol 5, 249 (2014).
77. Paul, S. & Schaefer, B. C. A new look at T cell receptor signaling to nuclear factor-kappaB. Trends Immunol 34, 269-281 (2013).
78. Qiao, Q. et al. Structural architecture of the CARMA1/Bc110/MALT1 signalosome: nucleation-induced filamentous assembly. Mol Cell 51, 766-779 (2013).
79. Lu, A. et al. Unified polymerization mechanism for the assembly of ASC-dependent inflammasomes. Cell 156, 1193-1206 (2014).
80. Cai, X. et al. Prion-like polymerization underlies signal transduction in antiviral immune defense and inflammasome activation. Cell 156, 1207-1222 (2014).
81. Villalba, M. et al. A novel functional interaction between Vav and PKCtheta is required for TCR-induced T cell activation. Immunity 12, 151-160 (2000).
82. Soares, H. et al. Regulated vesicle fusion generates signaling nanoterritories that control T cell activation at the immunological synapse. J Exp Med 210, 2415-2433 (2013).
83. Kachaner, D. et al. Plk1-dependent phosphorylation of optineurin provides a negative feedback mechanism for mitotic progression. Mol Cell 45, 553-566 (2012).
84. Heilemann, M. et al. Subdiffraction-resolution fluorescence imaging with conventional fluorescent probes. Angew Chem Int Ed Engl 47, 6172-6176 (2008).

1. A method of identifying a E-SYT2 modulator, comprising:
providing a cell that expresses E-SYT2;
contacting the cell with a candidate chemical entity; and
characterizing recruitment of at least one of Carma1, Bcl10, NEMO, and PKCθ to the immunological synapse (IS).

2. The method of claim 1, wherein recruitment of at least one of Carma1, Bcl10, NEMO, and PKCθ to the IS is reduced in the presence of the candidate chemical entity, and the candidate chemical entity is thereby identified as an inhibitor of E-SYT2 activity.

3. The method of claim 1, wherein the inhibitor of E-SYT2 activity binds to E-SYT2 protein to inhibit E-SYT2 protein function.

4. The method of claim 1, wherein the inhibitor of E-SYT2 activity inhibits expression of E-Syt2.

5. A method of inhibiting NF-κB activity in a cell comprising contacting the cell with an E-SYT2 inhibitor.

6. The method of claim 5, wherein contacting the cell with the E-SYT2 inhibitor reduces recruitment of at least one of Carma1, Bcl10, NEMO, and PKCθ to the immunological synapse (IS) in the T cell.

7. The method of claim 5, wherein the E-SYT2 inhibitor binds to E-SYT2 protein to inhibit E-SYT2 protein function.

8. The method of claim 5, wherein the E-SYT2 inhibitor inhibits expression of E-Syt2.

9. The method of claim 5, wherein the cell in cultured in vitro.

10. The method of claim 5, wherein the cell is in a patient and the method comprises administering the E-SYT2 inhibitor to the patient.

11. The method of claim 5, wherein NF-κB is constitutively active in the cell in the absence of the E-SYT2 inhibitor.

12. An E-SYT2 inhibitor identified by the method of claim 2.

13. A method comprising:
providing a sample from a patient suspected of having or at risk of having a MALT-lymphoma or an ABC-DLBCL-lymphoma; and
screening the sample to identify the presence and/or absence of a gain of function E-Syt2 mutation in the sample.

14. The method of claim 13, wherein if the sample comprises a gain of function E-Syt2 mutation the patient is diagnosed as having a MALT-lymphoma or a ABC-DLBCL-lymphoma.

15. The method of claim 13, wherein if the sample comprises a gain of function E-Syt2 mutation the patient is diagnosed as having an increased risk of having and/or developing a MALT-lymphoma or a ABC-DLBCL-lymphoma.

16. The method of claim 13, wherein the screening comprises analyzing a nucleic acid present in the sample.

17. The method of claim 16, wherein the screening comprises performing a hybridization and/or polymerization assay on the nucleic acid.

18. The method of claim 13, wherein the screening comprises analyzing the activity of E-SYT2 protein in the sample.

19. A genetically modified mammal comprising one or two loss of function alleles of E-Syt2, wherein the genetically modified mammal develops dermatitis.

20. A genetically modified mammal comprising two copies of a conditional loss of function allele of E-Syt2, wherein the conditional allele is recombined in the thymus of the genetically modified mammal such that the thymus of the mammal comprises cells that do not express functional E-SYT2.
